(19) United States
(12) Patent Application Publication
(10) Pub. No.: US 2008/0119451 A1
(43) Pub. Date: May 22, 2008

(54) Novel Benzamide Derivatives

(75) Inventors: Keith Hopkinson Gibson, Cheshire (GB); Elaine Sophie Elizabeth Stokes, Cheshire (GB); Michael James Waring, Cheshire (GB); David Michael Andrews, Cheshire (GB); Zbigniew Stanley Matusiak, Cheshire (GB)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (SE)

(21) Appl. No.: 11/814,340

(22) PCT Filed: Jan. 17, 2006
(86) PCT No.: PCT/GB06/00134

§ 371 Date: Jul. 19, 2007

(30) Foreign Application Priority Data
Jan. 20, 2005 (GB) 0501146.5

(51) Int. Cl. A61K 31/496; A61K 31/4439; C07D 403/02; C07D 211/84; C07D 213/56

(52) U.S. Cl. 514/210.2; 514/355; 514/343; 514/253.13; 544/360; 546/268.1; 546/279.1; 546/316

(57) ABSTRACT

The invention concerns benzamide derivatives of Formula (I) wherein R^{1a}, R^{1b}, R^{1c}, R², R³, R⁴, integer m, integer n and W have any of the meanings defined in the description; processes for their preparation, pharmaceutical compositions containing them and their use in the manufacture of a medicament for use as an antiproliferative agent in the prevention or treatment of tumours or other proliferative conditions which are sensitive to the inhibition of histone deacetylase (HDAC).

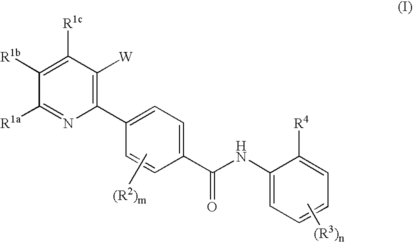

This invention relates to novel benzamide derivatives, or pharmaceutically acceptable salts or pro-drug forms thereof. The novel benzamide derivatives of the present invention are potent inhibitors of the enzyme histone deacetylase (HDAC), and are therefore useful agents for the treatment of disease states in which HDAC activity is known to be involved, such as cancer (Marks et al., Nature Reviews, 1, 194-202, (2001)), cystic fibrosis (Li, S. et al, J. Biol. Chem., 274, 7803-7815, (1999)), Huntingdons chorea (Steffan, J. S. et al., Nature, 413, 739-743, (2001)) and sickle cell anemia (Gabbianelli, M. et al., Blood, 95, 3555-3561, (2000)). Accordingly, the present invention relates to methods for the treatment of any of the aforementioned conditions in a warm-blooded animal, such as man, by administering a pharmacologically active amount of a benzamide derivative of the present invention. The present invention also relates to processes for the manufacture of the benzamide derivatives of the present invention, to pharmaceutical compositions comprising these benzamide derivatives, and to their use of these derivatives in the manufacture of medicaments to inhibit HDAC in a warm-blooded animal, such as man.

In the eukaryotic cell, DNA is routinely compacted to prevent transcription factor accessibility. When the cell is activated this compacted DNA is made available to DNA-binding proteins, thereby allowing the induction of gene transcription (Beato, M., J. Med. Chem., 74, 711-724 (1996); Wolffe, A. P., Nature, 387, 16-17 (1997)). Nuclear DNA is known to associate with proteins known as histones to form a complex that is known as chromatin. The core histones, termed H2A, H2B, H3 and H4, are surrounded by 146 base pairs of DNA to form the fundamental unit of chromatin, which is known as the nucleosome. The N-terminal tails of the core histones contain lysine residues that are sites for post-transcriptional acetylation. Acetylation of the terminal amino group on the lysine side chain neutralizes the potential of the side chain to form a positive charge, and is thought to impact on chromatin structure.

Histone Deacetylases (HDACs) are zinc-containing enzymes which catalyse the removal of acetyl groups from the ε-amino termini of lysine residues clustered near the amino terminus of nucleosomal histones. HDACs may be divided into two classes, the first (HDAC 1, 2, 3 and 8) represented by yeast Rpd3-like proteins, and the second (HDAC 4, 5, 6, 7, 9 and 10) represented by yeast Hdal-like proteins. The reversible process of acetylation is known to be important in transcriptional regulation and cell-cycle progression. In addition, HDAC deregulation has been associated with several cancers and HDAC inhibitors, such as Trichostatin A (a natural product isolated from Streptomyces hygroscopicus), have been shown to exhibit significant cell growth inhibition and anti-tumour effects (Meinke, P. T., Current Medicinal Chemistry, 8, 211-235 (2001)). Yoshida et al, (Exper. Cell Res., 177, 122-131 (1988)) teach that Trichostatin A causes the arrest of rat fibroblasts at the G1 and G2 phases of the cell cycle, thereby implicating the role of HDAC in the regulation of the cell cycle. Furthermore, Trichostatin A has been shown to induce terminal differentiation, inhibit cell growth, and prevent the formation of tumours in mice (Finnin et al., Nature, 401, 188-193 (1999)).

It is known from International Patent Publication Numbers WO 03/087057 and WO 03/092686, that certain benzamide derivatives are inhibitors of HDAC. One particular compound disclosed in WO 03/087057 is N-(2-aminophenyl)-4-pyridin-2-ylbenzamide.

However, there is no specific disclosure in either of these documents of benzamide derivatives which possess a further substituted-pyridin-2-yl group in the 4-position of the benzamide group. We have now found that certain benzamide derivatives possessing a substituted-pyridin-2-yl group in the 4-position are potent inhibitors of the HDAC enzyme.

According to the present invention there is provided a compound of formula (I):

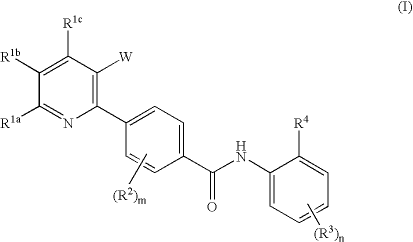

wherein:

### R^{1a} is selected from hydrogen, amino, (1-3C)alkyl, N-(1-3C)alkylamino, N,N-di-(1-3C)alkylamino, or a group of the sub-formula II:

R⁵R⁶N—X—[CR^{a}R^{b}]_{q}— (II)

wherein:
q is 1, 2 or 3;
each R^{a} and R^{b} group present is independently selected from hydrogen, halo, hydroxy or (1-4C)alkyl;
X¹ is selected from a direct bond or —C(O)—; and
R⁵ and R⁶ are each independently selected from hydrogen or (1-3C)alkyl;
and wherein if R^{1a} is a N-(1-3C)alkylamino or N,N-di-(1-3C)alkylamino group, the (1-3C)alkyl moiety is optionally substituted by hydroxy or (1-2C)alkoxy;
   R^{1b} is selected from:
(i) hydrogen, (1-6C)alkyl, halo(1-6C)alkyl, hydroxy(1-6C)alkyl, (3-6C)cycloalkyl, (3-6C)cycloalkyl(1-4C)alkyl, (1-6C)alkoxy, (1-6C)alkoxy(1-6C)alkyl, N-(1-4C)alkylsulphamoyl, N,N-di-[(1-4C)alkyl]sulphamoyl; or
(ii) a group of sub-formula III:

R⁷R⁸N—[CR^{a}R^{b}]ₐ—X²— (III)

wherein:
R⁷ and R⁸ are independently selected from hydrogen, (1-6C)alkyl, (3-6C)cycloalkyl, (3-6C)cycloalkyl(1-6C)alkyl, (1-6C)alkoxy(1-6C)alkyl, (2-3C)alkenyl, (2-3C)alkynyl, or a group of formula IV:

R⁹R¹⁰N—[CR^{a}R^{b}]_{b}—X³— (IV)

wherein:
b is 1, 2 or 3;
R^{a} and R^{b} are as defined above;
X³ is a direct bond or —C(O)—;
R⁹ and R¹⁰ are independently selected from hydrogen, (1-6C)alkyl, (3-6C)cycloalkyl, (3-6C)cycloalkyl(1-6C)alkyl, (1-6C)alkoxy(1-6C)alkyl, or R⁹ and R¹⁰ are linked so that, together with the nitrogen atom to which they are attached, they form a 4-, 5-, 6- or 7-membered heterocyclic ring, said heterocyclic ring optionally comprising, in addition to the nitrogen atom to which R⁹ and R¹⁰ are attached, one or two further heteroatoms selected from N, O or S, and wherein said heterocyclic ring is optionally substituted by one or more hydroxy, halo, (1-4C)alkyl, carbamoyl, oxo, —[CH₂]ₑ—NR¹¹R¹² (wherein e is 0, 1 or 2, and R¹¹ and R¹² are independently selected from hydrogen, (1-6C)alkyl, (3-6C)cycloalkyl or (3-6C)cycloalkyl(1-6C)alkyl) groups;
or R⁷ and R⁸ are linked so that, together with the nitrogen atom to which they are attached, they form a 4-, 5-, 6- or 7-membered heterocyclic ring, said heterocyclic ring optionally comprising, in addition to the nitrogen atom to which R⁷ and R⁸ are attached, one or two further heteroatoms selected from N, O or S, and wherein said heterocyclic ring is optionally substituted by one or more hydroxy, halo, (1-4C)alkyl, carbamoyl, oxo, (2-4C)alkenyl, (2-4C)alkynyl, (1-4C)alkoxy, (1-4C)alkoxy(1-4C)alkyl, (1-4C)alkyl-S(O)_{q}— (where q is 0, 1 or 2), a 5- or 6-membered heterocyclic ring comprising one to three heteroatoms selected from N, O or S, or —[CH₂]_{f}—NR¹³R¹⁴ groups (wherein f is 0, 1 or 2, and R¹³ and R¹⁴ are independently selected from hydrogen, (1-6C)alkyl, (3-6C)cycloalkyl or (3-6C)cycloalkyl(1-6C)alkyl);

X² is selected from a direct bond, —O— or —C(O)—, with the proviso that X² can only be —C(O)— if at least one of R⁷ or R⁸ is a group of formula IV as defined above;
a is 0, 1, 2, 3 or 4;
R^{a} and R^{b} are as defined above; or
(iii) a group of the sub-formula VII:

Q-Z-Y—

wherein:
Y is a direct bond or —[CR^{a}R^{b}]ₓ—, where integer x is 1 to 4 and R^{a} and R^{b} are as defined above;
Z is absent or selected from —O—, —S—, —SO—, —SO₂—, —NH—SO₂—, —SO₂—NH— or —C(O)—; and
Q is a carbon-linked heterocyclyl or a heterocyclyl-(1-6C)alkyl group, said heterocyclyl or a heterocyclyl-(1-6C)alkyl group being optionally substituted on the heterocyclyl ring by one or more substituent groups (for example 1, 2 or 3), which may be the same or different, selected from halo, oxo, cyano, hydroxy, trifluoromethyl, amino, carboxy, carbamoyl, mercapto, sulphamoyl, (1-3C)alkyl, (2-3C)alkenyl, (2-3C)alkynyl, (1-3C)alkoxy, (1-3C)alkanoyl, (1-3C)alkanoyloxy, (1-3C)alkoxy(1-3C)alkyl, (1-3C)alkoxycarbonyl, halo(1-3C)alkyl, N-[(1-3C)alkyl]amino, N,N-di-[(1-3C)alkyl]amino, N-[(1-3C)alkoxy(1-3C)alkyl]amino, N,N-di-[(1-3C)alkoxy(1-3C)alkyl]amino, N-[(1-3C)alkoxy(1-3C)alkyl]-N-[(1-3C)alkyl]amino, N-(1-3C)alkylcarbamoyl, N,N-di-[(1-3C)alkyl]carbamoyl, (1-3C)alkylthio, (1-3C)alkylsulphinyl, (1-3C)alkylsulphonyl, N-(1-3C)alkylsulphamoyl, N,N-di-[(1-3C)alkyl]sulphamoyl;
R^{1c} is selected from hydrogen, halo, cyano, hydroxy, trifluoromethyl, trifluoromethoxy, amino, carboxy, carbamoyl, mercapto, sulphamoyl, (1-3C)alkyl, (2-3C)alkenyl, (2-3C)alkynyl, (1-3C)alkoxy, (1-3C)alkanoyl, (1-3C)alkanoyloxy, N-(1-3C)alkylamino, N,N-di-[(1-3C)alkyl]amino, (1-3C)alkanoylamino, N-(1-3C)alkylcarbamoyl, N,N-di-(1-3C)alkylcarbamoyl, (1-3C)alkylthio, (1-3C)alkylsulphinyl, (1-3C)alkylsulphonyl, (1-3C)alkoxycarbonyl, N-(1-3C)alkylsulphamoyl, and N,N-di-(1-3C)alkylsulphamoyl;
integer m is 0, 1, 2, 3 or 4;

R² is halo;

integer n is 0, 1, 2, 3 or 4;

R³ is selected from halo, cyano, hydroxy, trifluoromethyl, trifluoromethoxy, amino, carboxy, carbamoyl, mercapto, sulphamoyl, (1-3C)alkyl, (2-3C)alkenyl, (2-3C)alkynyl, (1-3C)alkoxy, (1-3C)alkanoyl, (1-3C)alkanoyloxy, N-(1-3C)alkylamino, N,N-di-[(1-3C)alkyl]amino, (1-3C)alkanoylamino, N-(1-3C)alkylcarbamoyl, N,N-Di(1-3C)alkylcarbamoyl, (1-3C)alkylthio, (1-3C)alkylsulphinyl, (1-3C)alkylsulphonyl, (1-3C)alkoxycarbonyl, N-(1-3C)alkylsulphamoyl, and N,N-di-(1-3C)alkylsulphamoyl;

R⁴ is amino or hydroxy; and

W is methyl or ethyl;

or a pharmaceutically acceptable salt or pro-drug thereof.

It is to be understood that, insofar as certain of the compounds of Formula I defined above may exist in optically active or racemic forms by virtue of one or more asymmetric carbon atoms, the invention includes in its definition any such optically active or racemic form which possesses the above-mentioned activity. The synthesis of optically active forms may be carried out by standard techniques of organic chemistry well known in the art, for example by synthesis from optically active starting materials or by resolution of a racemic form. Similarly, the above-mentioned activity may be evaluated using the standard laboratory techniques referred to hereinafter.

It is to be understood that certain compounds of Formula I defined above may exhibit the phenomenon of tautomerism. In particular, tautomerism may affect heterocyclic groups within the R^{1b} groups that bear 1 or 2 oxo substituents. It is to be understood that the present invention includes in its definition any such tautomeric form, or a mixture thereof, which possesses the above-mentioned activity and is not to be limited merely to any one tautomeric form utilised within the formulae drawings or named in the Examples.

Where optional substituents are selected from “one or more” substituent groups it is to be understood that this definition includes all substituents being chosen from one of the specified groups or the substituents being chosen from two or more of the specified groups. In particular for example, where R⁹ and R¹⁰ together with the nitrogen atom to which they are attached form a heterocyclic ring, it may be optionally substituted by from 1 to 3, preferably 1 or 2 and most preferably one of the substituents listed above. Similarly when R⁷ and R⁸ are linked so that, together with the nitrogen atom to which they are attached, they heterocyclic ring, said heterocyclic ring is suitably optionally substituted by from 1 to 3, more suitably one or two and preferably one of the substituents listed above.

In this specification the generic term “(1-6C)alkyl” includes both straight-chain and branched-chain alkyl groups such as propyl, isopropyl and tert-butyl, and also (3-6C)cycloalkyl groups such as cyclopropyl, cyclobutyl, cyclopentyl, and cyclohexyl, and also cycloalkyl-alkyl groups having 4 to 6 carbon atoms, such as cyclopropylmethyl, 2-cyclopropylethyl, cyclobutylmethyl, 2-cyclobutylethyl, and cyclopentylmethyl. However, references to individual alkyl groups such as “propyl” are specific for the straight-chain version only, references to individual branched-chain alkyl groups such as “isopropyl” are specific for the branched-chain version only and references to individual cycloalkyl groups such as “cyclopentyl” are specific for that 5-membered ring only.

An analogous convention applies to other generic terms, for example (1-6C)alkoxy includes (3-6C)cycloalkyloxy groups and cycloalkyl-alkoxy groups having 4 to 6 carbon atoms, for example methoxy, ethoxy, propoxy, isopropoxy, cyclopropyloxy, cyclobutyloxy, cyclopentyloxy, cyclohexyloxy, cyclopropylmethoxy, 2-cyclopropylethoxy, cyclobutylmethoxy, 2-cyclobutylethoxy and cyclopentylmethoxy.

A person skilled in the art will appreciate that the terms “(1-4C)alkyl”, “(1-3C)alkyl” and “(1-2C)alkyl” are used herein refer to any of the alkyl groups defined above that posses 1 to 4, 1 to 3 and 1 to 2 carbon atoms respectively. The same convention applies to other terms used herein, such as, for example, “(1-4C)alkoxy”, “(1-3C)alkoxy” and “(1-2C)alkoxy”.

The term “halo” refers to fluoro, chloro, bromo and iodo.

The term “heterocyclyl”, unless otherwise defined herein, refers to a saturated, partially saturated or unsaturated, mono or bicyclic ring containing 3-12 atoms of which at least one atom is chosen from nitrogen, sulphur or oxygen, and which may, unless otherwise specified, be carbon or nitrogen linked, and wherein a CH₂ group can optionally be replaced by a C(O), and wherein a ring sulphur atom may be optionally oxidised to form the S-oxide(s). Preferably a “heterocyclyl” is a saturated, partially saturated or unsaturated, monocyclic ring containing 4, 5, 6 or 7 atoms, wherein at least one atom of the ring is chosen from nitrogen, sulphur or oxygen, and the ring system may, unless otherwise specified, be carbon or nitrogen linked, and wherein a ring sulphur atom may be optionally oxidised to form S-oxide(s). Examples and suitable values of the term “heterocyclyl” are azetidinyl, thiazolidinyl, pyrrolidinyl, 1,3-benzodioxolyl, 1,2,4-oxadiazolyl, morpholinyl, tetrahydrofuranyl, furanyl, tetrahydropyranyl, piperidinyl, piperazinyl, thiomorpholinyl, 1,3-dioxolanyl, homopiperidinyl, homopiperazinyl, thienyl, pyrrolyl, pyrazolyl, oxadiazolyl, tetrazolyl, oxazolyl, thienopyrimidinyl, thienopyridinyl, thieno[3,2d]pyrimidinyl, 1,3,5-triazinyl, isoxazolyl, imidazolyl, thiadiazolyl, isothiazolyl, 1,2,3-triazolyl, 1,2,4-triazolyl, pyranyl, pyrimidyl, thiazolyl, pyridazinyl, pyrazinyl and pyridyl. For instance, these may include are azetidinyl, thiazolidinyl, pyrrolidinyl, 1,3-benzodioxolyl, 1,2,4-oxadiazolyl, morpholinyl, tetrahydrofuranyl, furanyl, tetrahydropyranyl, piperidinyl, piperazinyl, thiomorpholinyl, 1,3-dioxolanyl, homopiperidinyl, homopiperazinyl, thienyl, pyrrolyl, pyrazolyl, oxadiazolyl, tetrazolyl, oxazolyl, thienopyrimidinyl, thienopyridinyl, thieno[3,2-d]pyrimidinyl, 1,3,5-triazinyl, isoxazolyl, imidazolyl, thiadiazolyl, isothiazolyl, 1,2,3-triazolyl, 1,2,4-triazolyl, pyranyl, pyrimidyl, thiazolyl, pyridazinyl, and pyridyl. Alternatively heterocycles may include pyrazinyl.

Particular examples of 4-, 5- or 6-membered monocyclic heterocyclyl groups include azetidinyl, pyrrolidinyl, piperidinyl, piperazinyl, morpholinyl, pyrrolyl, pyrazinyl and pyridyl, for instance, these may include azetidinyl, pyrrolidinyl, piperidinyl, piperazinyl, morpholinyl, pyrrolyl, and pyridyl. They may also include pyrazinyl. Particular examples of heterocycles include azetidin-1-yl, pyrrolidin-1-yl, piperidin-1-yl, piperazin-1-yl, morpholin-4-yl, pyrrol-1-yl, pyrid-1-yl, pyrid-2-yl, pyrid-3-yl and pyrid-4-yl. Another particular example is pyrazin-2-yl. Where a heterocyclyl group includes one or more nitrogen atoms, these may carry a hydrogen atom or a substituent group such as a (1-6C)alkyl group if required to fulfil the bonding requirements of nitrogen, or they may be linked to the rest of the structure by way of the nitrogen atom. A nitrogen atom within a heterocyclyl group may be oxidized to give the corresponding N oxide.

Within this specification composite terms are used to describe groups comprising more that one functionality, such as heterocyclyl-(1-6C)alkyl. These composite terms are to be given their ordinary meanings and will be understood by a person skilled in the art. For example, the term heterocyclyl(1-6C)alkyl refer to substituent groups wherein a heterocyclyl moiety is linked via a (1-6C)alkyl chain. The same convention also applies to other composite terms used herein, such as (1-6C)alkoxy(1-6C)alkyl and (3-6C)cycloalkyl(1-6C)alkyl.

Examples and suitable values of heterocyclic rings formed when either of groups R⁷ and R⁸ or R⁹ and R¹⁰ are linked include azetidin-1-yl, pyrrolidine-1-yl, piperidine-1-yl, homo-piperidine-1-yl, piperazin-1-yl, homo-piperazin-1-yl, morpholin-4-yl, thiomorpholin-4-yl, imidazolidin-1-yl, and pyrazolidin-1-yl.

In an embodiment of the invention, it is preferred that any heterocyclic ring present within a R^{1b} substituent group contains nitrogen as the only heteroatom present. Suitably, the heterocyclic ring contains one or two ring nitrogen atoms as the only heteroatoms present.

Within this specification composite terms are used to describe groups comprising more that one functionality, such as heterocyclyl-(1-6C)alkyl. These composite terms are to be given their ordinary meanings and will be understood by a person skilled in the art. For example, the term heterocyclyl(1-6C)alkyl refer to substituent groups wherein a heterocyclyl moiety is linked via a (1-6C)alkyl chain. The same convention also applies to other composite terms used herein, such as (1-6C)alkoxy(1-6C)alkyl and (3-6C)cycloalkyl(1-6C)alkyl.

Suitable values for groups in the definitions of any of R^{1a}, R^{1c}, or R³ are as follows:
for (1-3C)alkyl: methyl, ethyl, propyl, and isopropyl;
for (2-3C)alkenyl: vinyl, isopropenyl, and allyl;
for (2-3C)alkynyl: ethynyl, and 2-propynyl;
for (1-3C)alkanoyl: acetyl and propionyl;
for (1-3C)alkanoyloxy: acetoxy and propionyloxy;
for (1-3C)alkoxy: methoxy, ethoxy, and propoxy;
for N-(1-3C)alkylamino: methylamino, ethylamino, propylamino, and isopropylamino;
for N,N-di-[(1-3C)alkyl]amino: dimethylamino, diethylamino, N-ethyl-N-methylamino and diisopropylamino;
for (1-3C)alkanoylamino: acetamido and propionamido;
for N-(1-3C)alkylcarbamoyl: N-methylcarbamoyl, N-ethylcarbamoyl and N-propylcarbamoyl;
for N,N-di-(1-3C)alkylcarbamoyl: N,N-dimethylcarbamoyl, N-ethyl-N-methylcarbamoyl and N,N-diethylcarbamoyl;
for (1-3C)alkylthio: methylthio, ethylthio and propylthio;
for (1-3C)alkylsulphinyl: methylsulphinyl and ethylsulphinyl; for (1-3C)alkylsulphonyl: methylsulphonyl and ethylsulphonyl;
for (1-3C)alkoxycarbonyl: methoxycarbonyl, ethoxycarbonyl, and propoxycarbonyl;
for N-(1-3C)alkylsulphamoyl: N-methylsulphamoyl and N-ethylsulphamoyl; and
for N,N-di-(1-3C)alkylsulphamoyl: N,N-dimethylsulphamoyl.

Suitable values for R^{1a} when it is a group of sub-formula II:

R⁵R⁶N—X¹—[CR^{a}R^{b}]_{q}—

include (methylamino)methyl, (ethylamino)methyl, (propylamino)methyl, (isopropylamino)methyl, (cyclopropylamino)methyl, (dimethylamino)methyl, N-methylcarbamoyl, N-ethylcarbamoyl, N-propylcarbamoyl; N,N-dimethylcarbamoyl, N-ethyl-N-methylcarbamoyl and N,N-diethylcarbamoyl.

Suitable values for R^{1b}, or moieties within a R^{1b} substituent group, are as follows:
for halo: fluoro, chloro, bromo and iodo;
for (1-6C)alkyl: methyl, ethyl, propyl, isopropyl, tert-butyl, cyclobutyl, cyclohexyl, cyclopropylmethyl, cyclobutylmethyl and 2-cyclopropylethyl;
for (1-4C)alkyl: methyl, ethyl, propyl, isopropyl, tert-butyl, and cyclobutyl;
for (1-2C)alkyl: methyl and ethyl;
for (2-3C)alkenyl: vinyl, isopropenyl, and allyl;
for (2-3C)alkynyl: ethynyl, and 2-propynyl;
for (3-6C)cycloalkyl: cyclopropyl, cyclobutyl, cyclopentyl, and cyclohexyl;
for (3-6C)cycloalkyl-(1-6C)alkyl: cyclopropylmethyl, cyclobutylmethyl, cyclopentyl methyl, 2-cyclopropylethyl and 2-cyclobutylpropyl;
for (1-6C)alkoxy: methoxy, ethoxy, propoxy, isopropoxy and butoxy;
for (1-6C)alkoxy(1-6C)alkyl: methoxymethyl, 2-methoxyethyl, 1-methoxyethyl, and 3-methoxypropyl;
for (1-3C)alkanoyl: acetyl and propionyl;
for (1-3C)alkanoyloxy: acetoxy and propionyloxy;
for (1-3C)alkoxycarbonyl: methoxycarbonyl and ethoxycarbonyl:
for halo(1-6C)alkyl: chloromethyl, 2-fluoro ethyl, 2-chloroethyl, 1-chloro ethyl, 2,2-difluoro ethyl, 2,2,2-trifluoro ethyl, 3-fluoropropyl, 3-chloropropyl, 3,3-difluoropropyl, 3,3,3-trifluoropropyl, and trifluoromethyl;
for hydroxy(1-6C)alkyl: hydroxymethyl, 2-hydroxyethyl, 1-hydroxyethyl and 3-hydroxypropyl;
for N-(1-3C)alkylamino: methylamino, ethylamino, propylamino, and isopropylamino;
for N,N-di-[(1-3C)alkyl]amino: dimethylamino, diethylamino, N-ethyl-N-methylamino and diisopropylamino;
for N-[(1-3C)alkoxy(1-3C)alkyl]amino: methoxymethylamino and 2-methoxyethylamino;
for N,N-di-[(1-3C)alkoxy(1-3C)alkyl]amino: di-methoxymethylamino and di-(2-methoxyethyl)amino;
for N-[(1-3C)alkoxy(1-3C)alkyl]-N-[(1-3C)alkyl]amino: N-(2-methoxyethyl)-N-methylamino, N-(2-methoxyethyl)-N-ethylamino and N-(2-methoxyethyl)-N-isopropylamino;
for (1-3C)alkylthio: methylthio, ethylthio and propylthio;
for (1-3C)alkylsulphinyl: methylsulphinyl and ethylsulphinyl;
for (1-3C)alkylsulphonyl: methylsulphonyl and ethylsulphonyl;
for (1-3C)alkoxycarbonyl: methoxycarbonyl, ethoxycarbonyl, and propoxycarbonyl;
for N-(1-3C)alkylcarbamoyl: N-methylcarbamoyl, N-ethylcarbamoyl and N-propylcarbamoyl;
for N,N-di-[(1-3C)alkyl]carbamoyl: N,N-dimethylcarbamoyl, N-ethyl-N-methylcarbamoyl and N,N-diethylcarbamoyl;
for N-(1-3C)alkylsulphamoyl: N-methylsulphamoyl and N-ethylsulphamoyl;
for N,N-di-[(1-3C)alkyl]sulphamoyl: N,N-dimethylsulphamoyl.

Examples of R^{1b} when it is a group of the sub-formula III:

R⁷R⁸N—[CR^{a}R^{b}]ₐ—X²—

include amino, (methylamino)methyl, (ethylamino)methyl, (propylamino)methyl, (isopropylamino)methyl, (cyclopropylamino)methyl, (butylamino)methyl, (cyclobutylamino)methyl, (cyclopentylamino)methyl, (1-methylpropylamino)methyl, (2-methylpropylamino)methyl, 1-(methylamino)ethyl, 2-(methylamino)ethyl, 2-(ethylamino)ethyl, 3-(methylamino)propyl, (di-methylamino)methyl, (di-ethylamino)methyl, [(ethyl)(methyl)amino]methyl, [(isopropyl)(methyl)amino]methyl, [(propyl)(methyl)amino]methyl, [(cyclopropylmethyl)amino]methyl, [(cyclobutylmethyl)(methyl)amino]methyl, [(2-methoxyethyl)(methyl)amino]methyl, [(isopropyl)(2-methoxyethyl)amino]methyl, [(2-methoxyethyl)amino]methyl, [(ethyl)(2-methoxyethyl)amino]methyl, {[2-(di-methylamino)ethyl]amino}methyl, {[2-(di-ethylamino)ethyl]amino}methyl, {[2-(di-methylamino)ethyl][methyl]amino}methyl, {[2-(di-ethylamino)ethyl][methyl]amino}methyl, [(2-pyrrolidin-1-ylethyl)amino]methyl, azetidin-1-ylmethyl, pyrrolidin-1-ylmethyl, [3-(dimethylamino)pyrrolidin-1-yl]methyl, (4-methylpiperazin-1-yl)methyl, (4-ethylpiperazin-1-yl)methyl, (4-isopropylpiperazin-1-yl)methyl, and [(2-methoxyethyl)piperazin-1-yl]methyl, [3-(methylsulfonyl)pyrrolidin-1-yl]methyl, (4-prop-2-yl-1-ylpiperazin-1-yl)methyl, (prop-2-yn-1-ylamino)methyl, [(prop-2-yn-1-yl)(methyl)amino]methyl, (4-pyrazin-2-ylpiperazin-1-yl)methyl, [(3-ethoxypropyl)amino]methyl, [(2-propoxyethyl)amino]methyl, [(2-methoxy-2-methylpropyl)amino]methyl, [(2-methoxyethyl)amino]methyl, [(3-methoxypropyl)amino]methyl, [(2-ethoxyethyl)amino]methyl, [(3-isopropoxypropyl)amino]methyl, [(2-methoxy-1-methylethyl)amino]methyl, and [(3-pyrrolidin-1-ylpropanoyl)amino].

Particular examples of R^{1b} include hydrogen, amino, (4-isopropylpiperazin-1-yl)methyl, (cyclopropylmethyl)amino]methyl, (4-ethylpiperazin-1-yl)methyl, [(2-pyrrolidin-1-ylethyl)amino]carbonyl, [2-(di-methylamino)ethyl]amino}carbonyl, [2-(di-methylamino)ethyl][methyl]amino}carbonyl, {[2-(di-ethylamino)ethyl]amino}carbonyl, pyrrolidin-1-ylmethyl, (methylamino)methyl, (ethylamino)methyl, (propylamino)methyl, (isopropylamino)methyl, (cyclopropylamino)methyl, (butylamino)methyl, (cyclobutylamino)methyl, (2-pyrrolidin-1-ylethyl)amino]methyl, [3-(dimethylamino)pyrrolidin-1-yl]methyl, (4-methylpiperazin-1-yl)methyl, [(2-methoxyethyl)piperazin-1-yl]methyl, [3-(methylsulfonyl)pyrrolidin-1-yl]methyl, (4-prop-2-yn-1-ylpiperazin-1-yl)methyl, (prop-2-yn-1-ylamino)methyl, [(prop-2-yn-1-yl)(methyl)amino]methyl, (4-pyrazin-2-ylpiperazin-1-yl)methyl, [(3-ethoxypropyl)amino]methyl, [(2-propoxyethyl)amino]methyl, [(2-methoxy-2-methylpropyl)amino]methyl, [(2-methoxyethyl)amino]methyl, [(3-methoxypropyl)amino]methyl, [(2-ethoxyethyl)amino]methyl, [(3-isopropoxypropyl)amino]methyl, [(2-methoxy-1-methylethyl)amino]methyl, and [(3-pyrrolidin-1-ylpropanoyl)amino].

Suitable examples of Q (sub-formula VII) when it is a heterocyclyl(1-6C)alkyl group include azetidinylmethyl, pyrrolidinylmethyl, 1-methyl-pyrrolidin-2-ylmethyl, piperidinylmethyl, piperazinylmethyl, morpholinylmethyl, 1-azetidinylethyl, 1-pyrrolidinylethyl, 1-piperidinylethyl, 1-piperazinylethyl, 1-morpholinylethyl.

When Q is a heterocyclyl-(1-6C)alkyl group it is preferable that it is a heterocyclyl-(1-4C)alkyl group, and more preferably a heterocyclyl-(1-2C)alkyl, and especially a heterocyclyl-methyl group.

A suitable pharmaceutically-acceptable salt of a compound of the Formula I is, for example, an acid-addition salt of a compound of the Formula I, for example an acid-addition salt with an inorganic or organic acid such as hydrochloric, hydrobromic, sulphuric, trifluoroacetic, citric or maleic acid; or, for example, a salt of a compound of the Formula I which is sufficiently acidic, for example an alkali or alkaline earth metal salt such as a calcium or magnesium salt, or an ammonium salt, or a salt with an organic base such as methylamine, dimethylamine, trimethylamine, piperidine, morpholine or tris-(2-hydroxyethyl)amine. A further suitable pharmaceutically-acceptable salt of a compound of the Formula I is, for example, a salt formed within the human or animal body after administration of a compound of the Formula I.

The compounds of the invention may be administered in the form of a pro-drug (that is a compound that is broken down in the human or animal body to release a compound of the invention). A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable ester derivatives that may be formed at a carboxy group or a hydroxy group in a compound of the Formula I and in vivo cleavable amide derivatives that may be formed at a carboxy group or an amino group in a compound of the Formula I.

Accordingly, the present invention includes those compounds of the Formula I as defined hereinbefore when made available by organic synthesis and when made available within the human or animal body by way of cleavage of a pro-drug thereof. Accordingly, the present invention includes those compounds of the Formula I that are produced by organic synthetic means and also such compounds that are produced in the human or animal body by way of metabolism of a precursor compound, that is a compound of the Formula I may be a synthetically-produced compound or a metabolically-produced compound.

A suitable pharmaceutically-acceptable pro-drug of a compound of the Formula I is one that is based on reasonable medical judgement as being suitable for administration to the human or animal body without undesirable pharmacological activities and without undue toxicity.

Various forms of pro-drug have been described, for example in the following documents:—
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

A suitable pharmaceutically-acceptable pro-drug of a compound of the Formula I that possesses a carboxy group is, for example, an in vivo cleavable ester thereof. An in vivo cleavable ester of a compound of the Formula I containing a carboxy group is, for example, a pharmaceutically-acceptable ester which is cleaved in the human or animal body to produce the parent acid. Suitable pharmaceutically-acceptable esters for carboxy include (1-6C)alkyl esters such as methyl, ethyl and tert-butyl, (1-6C)alkoxymethyl esters such as methoxymethyl esters, (1-6C)alkanoyloxymethyl esters such as pivaloyloxymethyl esters, 3-phthalidyl esters, (3-8C)cycloalkylcarbonyloxy-(1-6C)alkyl esters such as cyclopentylcarbonyloxymethyl and 1-cyclohexylcarbonyloxyethyl esters, 2-oxo-1,3-dioxolenylmethyl esters such as 5-methyl-2-oxo-1,3-dioxolen-4-ylmethyl esters and (1-6C)alkoxycarbonyloxy-(1-6C)alkyl esters such as methoxycarbonyloxymethyl and 1-methoxycarbonyloxyethyl esters.

A suitable pharmaceutically-acceptable pro-drug of a compound of the Formula I that possesses a hydroxy group is, for example, an in vivo cleavable ester or ether thereof. An in vivo cleavable ester or ether of a compound of the Formula I containing a hydroxy group is, for example, a pharmaceutically-acceptable ester or ether which is cleaved in the human or animal body to produce the parent hydroxy compound. Suitable pharmaceutically-acceptable ester forming groups for a hydroxy group include inorganic esters such as phosphate esters (including phosphoramidic cyclic esters). Further suitable pharmaceutically-acceptable ester forming groups for a hydroxy group include (1-10C)alkanoyl groups such as acetyl, benzoyl, phenylacetyl and substituted benzoyl and phenylacetyl groups, (1-10C)alkoxycarbonyl groups such as ethoxycarbonyl, N,N-[di-(1-4C)alkyl]carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(1-4C)alkylpiperazin-1-ylmethyl. Suitable pharmaceutically-acceptable ether forming groups for a hydroxy group include α-acyloxyalkyl groups such as acetoxymethyl and pivaloyloxymethyl groups.

A suitable pharmaceutically-acceptable pro-drug of a compound of the Formula I that possesses a carboxy group is, for example, an in vivo cleavable amide thereof, for example an amide formed with an amine such as ammonia, a (1-4C)alkylamine such as methylamine, a di-(1-4C)alkylamine such as dimethylamine, N-ethyl-N-methylamine or diethylamine, a (1-4C)alkoxy-(2-4C)alkylamine such as 2-methoxyethylamine, a phenyl-(1-4C)alkylamine such as benzylamine and amino acids such as glycine or an ester thereof.

A suitable pharmaceutically-acceptable pro-drug of a compound of the Formula I that possesses an amino group is, for example, an in vivo cleavable amide derivative thereof. Suitable pharmaceutically-acceptable amides from an amino group include, for example an amide formed with (1-10C)alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(1-4C)alkylpiperazin-1-ylmethyl.

The in vivo effects of a compound of the Formula I may be exerted in part by one or more metabolites that are formed within the human or animal body after administration of a compound of the Formula I. As stated hereinbefore, the in vivo effects of a compound of the Formula I may also be exerted by way of metabolism of a precursor compound (a pro-drug).

Particular novel compounds of the invention include, for example, benzamide derivatives of the Formula I, or pharmaceutically-acceptable salts or pro-drugs thereof, wherein, unless otherwise stated, each of R^{1a}, R^{1b}, R^{1c}, m, R², n, R³, and R⁴ has any of the meanings defined hereinbefore or in paragraphs (1) to (50) hereinafter:—
(1) R^{1a} is selected from hydrogen, amino, (1-3C)alkyl, N-(1-3C)alkylamino, and N,N-di-(1-3C)alkylamino;
   and wherein if R^{1a} is a N-(1-3C)alkylamino or N,N-di-(1-3C)alkylamino group, the (1-3C)alkyl moiety is optionally substituted by hydroxy or (1-2C)alkoxy;
(2) R¹¹ is selected from hydrogen, amino, (1-2C)alkyl, N-(1-2C)alkylamino, and N,N-di-(1-2C)alkylamino;
(3) R^{1a} is hydrogen;
(4) R^{1b} is selected from hydrogen, (1-4C)alkyl, halo(1-4C)alkyl, hydroxy(1-4C)alkyl, (3-4C)cycloalkyl, (3-4C)cycloalkyl(1-2C)alkyl, (1-4C)alkoxy, (1-4C)alkoxy(1-4C)alkyl, N-(1-4C)alkylsulphamoyl, N,N-di-[(1-4C)alkyl]sulphamoyl; or

a group of sub-formula III:

R⁷R⁸N—[CR^{a}R^{b}]ₐ—X²— (III)

wherein:
X² is selected from a direct bond, or —O—;
a is 0, 1, 2 or 3;
each R^{a} and R^{b} group present is independently selected from hydrogen or (1-2C)alkyl;
R⁷ and R⁸ are independently selected from hydrogen, (1-4C)alkyl, (3-4C)cycloalkyl, (3-4C)cycloalkyl(1-2C)alkyl, (1-4C)alkoxy(1-4C)alkyl, or a group of sub-formula IVA:

R⁹R¹⁰N—[CR^{a}R^{b}]_{b}— (IVA)

wherein:
b is 1, 2 or 3;
R^{a} and R^{b} are as defined above;
R⁹ and R¹⁰ are independently selected from hydrogen, (1-4C)alkyl, (3-4C)cycloalkyl, (3-4C)cycloalkyl(1-2C)alkyl, (1-4C)alkoxy(1-4C)alkyl, or R⁹ and R¹⁰ are linked so that, together with the nitrogen atom to which they are attached, they form a 4-, 5-, or 6-membered heterocyclic ring, wherein said heterocyclic ring optionally comprises, in addition to the nitrogen atom to which R⁹ and R¹⁰ are attached, an additional nitrogen atom, and the heterocyclic ring is optionally substituted by hydroxy, (1-2C)alkyl, oxo, or —[CH₂]ₑ—NR¹¹R¹² (wherein e is 0 or 1, and R¹¹ and R¹² are independently selected from hydrogen, or (1-4C)alkyl);
or R⁷ and R⁸ are linked so that, together with the nitrogen atom to which they are attached, they form a 4-, 5-, or 6-membered heterocyclic ring, wherein said heterocyclic ring optionally comprises, in addition to the nitrogen atom to which R⁷ and R⁸ are attached, an additional nitrogen atom, and the heterocyclic ring is optionally substituted by hydroxy, (1-4C)alkyl, oxo, or —[CH₂]_{f}—NR¹³R¹⁴ (wherein f is 0 or 1, and R¹³ and R¹⁴ are independently selected from hydrogen, or (1-4C)alkyl);
(5) R^{1b} is selected from hydrogen, (1-2C)alkyl, halo(1-2C)alkyl, hydroxy(1-2C)alkyl, (3-4C)cycloalkyl, (3-4C)cycloalkyl(1-2C)alkyl, (1-2C)alkoxy, (1-2C)alkoxy(1-2C)alkyl, N-(1-2C)alkylsulphamoyl, N,N-di-[(1-2C)alkyl]sulphamoyl; or

a group of sub-formula III:

R⁷R⁸N—[CR^{a}R^{b}]ₐ—X²— (III)

wherein:
X² is selected from a direct bond, or —O—;
a is 0, 1, or 2;
each R^{a} and R^{b} group present is independently selected from hydrogen;
R⁷ and R⁸ are independently selected from hydrogen, (1-4C)alkyl, (3-4C)cycloalkyl, (3-4C)cycloalkyl(1-2C)alkyl, (1-2C)alkoxy(1-2C)alkyl, or a group of sub-formula IVA:

R⁹R¹⁰N—[CR^{a}R^{b}]_{b}— (IVA)

wherein:
b is 2 or 3;
R^{a} and R^{b} are as defined above;
R⁹ and R¹⁰ are independently selected from hydrogen, (1-2C)alkyl, (3-4C)cycloalkyl, (3-4C)cycloalkyl(1-2C)alkyl, (1-2C)alkoxy(1-2C)alkyl, or R⁹ and R¹⁰ are linked so that, together with the nitrogen atom to which they are attached, they form a 4-, 5-, or 6-membered heterocyclic ring, wherein said heterocyclic ring optionally comprises, in addition to the nitrogen atom to which R⁹ and R¹⁰ are attached, an additional nitrogen atom, and the heterocyclic ring is optionally substituted by hydroxy, (1-2C)alkyl, oxo, or —[CH₂]ₑ—NR¹¹R¹² (wherein e is 0 or 1, and R¹¹ and R¹² are independently selected from hydrogen, or (1-2C)alkyl);
or R⁷ and R⁸ are linked so that, together with the nitrogen atom to which they are attached, they form a 4-, 5-, or 6-membered heterocyclic ring, wherein said heterocyclic ring optionally comprises, in addition to the nitrogen atom to which R⁷ and R⁸ are attached, an additional nitrogen atom, and the heterocyclic ring is optionally substituted by hydroxy, (1-4C)alkyl, oxo, or —[CH₂]_{f}—NR¹³R¹⁴ (wherein f is 0 or 1, and R¹³ and R¹⁴ are independently selected from hydrogen, or (1-2C)alkyl);
(6) R^{1b} is selected from hydrogen, (1-2C)alkyl, or hydroxy(1-2C)alkyl;

or a group of sub-formula III:

R⁷R⁸N—[CR^{a}R^{b}]ₐ—X²— (II)

wherein:
X² is selected from a direct bond or —O—;
a is 1 or 2;
each R^{a} and R^{b} group present is hydrogen;
R⁷ and R⁸ are independently selected from hydrogen, (1-4C)alkyl, (3-4C)cycloalkyl, (3-4C)cycloalkyl(1-2C)alkyl, or a group of sub-formula IVA:

R⁹R¹⁰N—[CR^{a}R^{b}]_{b}— (IVA)
wherein:
   b is 2 or 3;
   R^{a} and R^{b} are as defined above;
   R⁹ and R¹⁰ are independently selected from hydrogen, (1-2C)alkyl, or
   R⁹ and R¹⁰ are linked so that, together with the nitrogen atom to which they are attached, they form a 4-, 5-, or 6-membered heterocyclic ring, wherein the nitrogen atom to which R⁹ and R¹⁰ are attached is the only heteroatom present in the ring;
   or R⁷ and R⁸ are linked so that, together with the nitrogen atom to which they are attached, they form a 4-, 5-, or 6-membered heterocyclic ring, wherein said heterocyclic ring optionally comprises, in addition to the nitrogen atom to which R⁷ and R⁸ are attached, an additional nitrogen atom, and the heterocyclic ring is optionally substituted by (1-4C)alkyl or —[CH₂]_{f}—NR¹³R¹⁴ (wherein f is 0, and R¹³ and R¹⁴ are independently selected from hydrogen, or (1-2C)alkyl);
(7) R^{1b} is selected from hydrogen, (1-4C)alkyl, hydroxy(1-4C)alkyl,

or a group of sub-formula III:

R⁷R⁸N—[CR^{a}R^{b}]ₐ—X²— (III)
wherein:
   X² is a direct bond or —O—;
   a is 1 or 2;
   R^{a} and R^{b} are both hydrogen;
   R⁷ and R⁸ are independently selected from hydrogen, (1-4C)alkyl, (3-4C)cycloalkyl, (3-4C)cycloalkyl(1-2C)alkyl, or a group of formula IVA:

R⁹R¹⁰N—[CR^{a}R^{b}]_{b}— (IVA)
wherein:
   b is 2 or 3;
   R^{a} and R^{b} are as defined above;
   R⁹ and R¹⁰ are independently selected from hydrogen, (1-4C)alkyl, or
   R⁹ and R¹⁰ are linked so that, together with the nitrogen atom to which they are attached, they form a 4-, 5- or 6-membered heterocyclic ring, and wherein the nitrogen atom to which R⁹ and R¹⁰ are attached is the only heteroatom present in the ring;
   or R⁷ and R⁸ are linked so that, together with the nitrogen atom to which they are attached, they form a 4-, 5- or 6-membered heterocyclic ring, which is optionally substituted by (1-4C)alkyl or —[CH₂]_{f}—NR¹³R¹⁴ (wherein f is 0 or 1, and R¹³ and R¹⁴ are independently selected from hydrogen, or (1-2C)alkyl), and wherein the nitrogen atom to which R⁹ and R¹⁰ are attached is the only heteroatom present in the ring;
(8) R^{1b} is selected from hydrogen or a group of sub-formula III:

R⁷R⁸N—[CR^{a}R^{b}]ₐ—X²— (III)

wherein:
X² is selected from a direct bond or —O—;
a is 1 or 2;
R^{a} and R^{b} are both hydrogen;
R⁷ and R⁸ are independently selected from hydrogen, (1-4C)alkyl, (3-4C)cycloalkyl, (3-4C)cycloalkyl(1-2C)alkyl, or a group of sub-formula IVA:

R⁹R¹⁰N—[CR^{a}R^{b}]_{b}— (IVA)

wherein:
b is 2 or 3;
R^{a} and R^{b} are as defined above;
R⁹ and R¹⁰ are independently selected from hydrogen, (1-2C)alkyl, or R⁹ and R¹⁰ are linked so that, together with the nitrogen atom to which they are attached, they form a 4-, 5-, or 6-membered heterocyclic ring, wherein the nitrogen atom to which R⁹ and R¹⁰ are attached is the only heteroatom present in the ring;
or R⁷ and R⁸ are linked so that, together with the nitrogen atom to which they are attached, they form a 4-, 5-, or 6-membered heterocyclic ring, wherein said heterocyclic ring optionally comprises, in addition to the nitrogen atom to which R⁷ and R⁸ are attached, an additional nitrogen atom, and the heterocyclic ring is optionally substituted by (1-3C)alkyl or —[CH₂]_{f}—NR¹³R¹⁴ (wherein f is 0 or 1, and R¹³ and R¹⁴ are independently selected from hydrogen, or (1-2C)alkyl);
(9) R^{1b} is selected from hydrogen, or a group of sub-formula III:

R⁷R⁸N—[CR^{a}R^{b}]ₐ—X²— (III)

wherein:
X² is a direct bond;
a is 1;
R^{a} and R^{b} are both hydrogen;
R⁷ and R⁸ are independently selected from hydrogen, methyl, ethyl, propyl, isopropyl, butyl, cyclopropyl, cyclobutyl, cyclopropylmethyl, or a group of sub-formula IVA:

R⁹R¹⁰N—[CR^{a}R^{b}]_{b}— (IVA)

wherein:
b is 1 or 2;
R^{a} and R^{b} are as defined above;
R⁹ and R¹⁰ are independently selected from hydrogen, methyl, ethyl, propyl or R⁹ and R¹⁰ are linked so that, together with the nitrogen atom to which they are attached, they form an azetidinyl, pyrrolidinyl, or piperidinyl ring;
or R⁷ and R⁸ are linked so that, together with the nitrogen atom to which they are attached, they form a heterocyclic ring selected from azetidinyl, pyrrolidinyl, piperidinyl, and piperazinyl, and wherein said heterocyclic ring is optionally substituted by methyl, ethyl, propyl, isopropyl or dimethylamino;
(10) R^{1b} is selected from hydrogen, (methylamino)methyl, (ethylamino)methyl, (propylamino)methyl, (isopropylamino)methyl, (cyclopropylamino)methyl, (butylamino)methyl, (cyclobutylamino)methyl, (cyclopentylamino)methyl, (1-methylpropylamino)methyl, (2-methylpropylamino)methyl, 1-(methylamino)ethyl, 2-(methylamino)ethyl, 2-(ethylamino)ethyl, (di-methylamino)methyl, (di-ethylamino)methyl, [(ethyl)(methyl)amino]methyl, [(isopropyl)(methyl)amino]methyl, [(propyl)(methyl)amino]methyl, [(cyclopropylmethyl)amino]methyl, [(cyclobutylmethyl)(methyl)amino]methyl, [(2-methoxyethyl)(methyl)amino]methyl, [(isopropyl)(2-methoxyethyl)amino]methyl, [(2-methoxyethyl)amino]methyl, [(ethyl)(2-methoxyethyl)amino]methyl, [(2-hydroxyethyl)(ethyl)amino]methyl, [(2-hydroxyethyl)(methyl)amino]methyl, {[2-(di-methylamino)ethyl]amino}methyl, {[2-(di-ethylamino)ethyl]amino}methyl, {[2-(di-methylamino)ethyl][methyl]amino}methyl, {[2-(di-ethylamino)ethyl][methyl]amino}methyl, [(2-pyrrolidin-1-ylethyl)amino]methyl, azetidin-1-ylmethyl, pyrrolidin-1-ylmethyl, [3-(dimethylamino)pyrrolidin-1-yl]methyl, piperidinylmethyl, piperazinylmethyl, (4-methylpiperazin-1-yl)methyl, (4-ethylpiperazin-1-yl)methyl, (4-isopropylpiperazin-1-yl)methyl;
(11) R^{1b} is selected from hydrogen, (methylamino)methyl, (ethylamino)methyl, (propylamino)methyl, (isopropylamino)methyl, (butylamino)methyl, (cyclobutylamino)methyl, (2-methylpropylamino)methyl, [(cyclopropylmethyl)amino]methyl, [(2-pyrrolidin-1-ylethyl)amino]methyl, azetidin-1-ylmethyl, pyrrolidin-1-ylmethyl, [3-(dimethylamino)pyrrolidin-1-yl]methyl, (4-methylpiperazin-1-yl)methyl, (4-ethylpiperazin-1-yl)methyl, (4-isopropylpiperazin-1-yl)methyl;
(12) R^{1b} is hydrogen or a group of sub-formula III:

R⁷R⁸N—[CR^{a}R^{b}]ₐ—X²— (III)

wherein:
X² is selected from a direct bond, —O— or —C(O)—;
a is 0, 1, 2, 3 or 4;
R^{a} and R^{b} are both hydrogen or (1-2C)alkyl;
R⁷ and R⁸ are independently selected from hydrogen, (1-6C)alkyl, (3-6C)cycloalkyl, (3-6C)cycloalkyl(1-6C)alkyl, (1-6C)alkoxy(1-6C)alkyl, or a group of formula IVA:

R⁹R¹⁰N—[CR^{a}R^{b}]_{b}— (IVA)

wherein:
b is 1, 2 or 3;
R^{a} and R^{b} are as defined above;
R⁹ and R¹⁰ are independently selected from hydrogen, (1-6C)alkyl, (3-6C)cycloalkyl, (3-6C)cycloalkyl(1-6C)alkyl, (1-6C)alkoxy(1-6C)alkyl, or R⁹ and R¹⁰ are linked so that, together with the nitrogen atom to which they are attached, they form a 4-, 5-, 6- or 7-membered heterocyclic ring, said heterocyclic ring optionally comprising, in addition to the nitrogen atom to which R⁹ and R¹⁰ are attached, one or two heteroatoms selected from N, O or S, and wherein the heterocyclic ring is optionally substituted by hydroxy, halo, (1-4C)alkyl, carbamoyl, oxo, or —[CH₂]ₑ—NR¹¹R¹² (wherein e is 0, 1 or 2, and R¹¹ and R¹² are independently selected from hydrogen, (1-6C)alkyl, (3-6C)cycloalkyl or (3-6C)cycloalkyl(1-6C)alkyl);
or R⁷ and R⁸ are linked so that, together with the nitrogen atom to which they are attached, they form a 4-, 5-, 6- or 7-membered heterocyclic ring, said heterocyclic ring optionally comprising, in addition to the nitrogen atom to which R⁷ and R⁸ are attached, one or two heteroatoms selected from N, O or S, and wherein the heterocyclic ring is optionally substituted by hydroxy, halo, (1-4C)alkyl, carbamoyl, oxo, or —[CH₂]_{f}—NR¹³R¹⁴ (wherein f is 0, 1 or 2, and R¹³ and R¹⁴ are independently selected from hydrogen, (1-6C)alkyl, (3-6C)cycloalkyl or (3-6C)cycloalkyl(1-6C)alkyl);
(13) R^{1b} is hydrogen or a group of sub-formula III:

R⁷R⁸N—[CR^{a}R^{b}]ₐ—X²— (III)

wherein:
X² is selected from a direct bond, or —O—;
a is 0, 1, 2, or 3;
R^{a} and R^{b} are both hydrogen;
R⁷ and R⁸ are independently selected from hydrogen, (1-4C)alkyl, (3-6C)cycloalkyl, (3-6C)cycloalkyl(1-4C)alkyl, (1-4C)alkoxy(1-4C)alkyl, or a group of formula IVA:

R⁹R¹⁰N—[CR^{a}R^{b}]_{b}— (IVA)

wherein:
b is 2 or 3;
R^{a} and R^{b} are as defined above;
R⁹ and R¹⁰ are independently selected from hydrogen, (1-4C)alkyl, (3-6C)cycloalkyl, (3-6C)cycloalkyl(1-4C)alkyl, (1-4C)alkoxy(1-4C)alkyl, or R⁹ and R¹⁰ are linked so that, together with the nitrogen atom to which they are attached, they form a 4-, 5-, or 6-membered heterocyclic ring, said heterocyclic ring optionally comprising, in addition to the nitrogen atom to which R⁹ and R¹⁰ are attached, one or two further nitrogen atoms, and wherein the heterocyclic ring is optionally substituted by hydroxy, halo, (1-4C)alkyl, carbamoyl, oxo, or —[CH₂]ₑ—NR¹¹R¹² (wherein e is 0, 1 or 2, and R¹¹ and R¹² are independently selected from hydrogen, (1-4C)alkyl, (3-6C)cycloalkyl or (3-6C)cycloalkyl(1-2C)alkyl);
or R⁷ and R⁸ are linked so that, together with the nitrogen atom to which they are attached, they form a 4-, 5-, or 6-membered heterocyclic ring, said heterocyclic ring optionally comprising, in addition to the nitrogen atom to which R⁷ and R⁸ are attached, one or two further nitrogen atoms, and wherein the heterocyclic ring is optionally substituted by hydroxy, halo, (1-4C)alkyl, carbamoyl, oxo, or —[CH₂]_{f}—NR¹³R¹⁴ (wherein f is 0, 1 or 2, and R¹³ and R¹⁴ are independently selected from hydrogen, (1-4C)alkyl, (3-6C)cycloalkyl or (3-6C)cycloalkyl(1-2C)alkyl);
(14) R^{1b} is hydrogen or a group of sub-formula III:

R⁷R⁸N—[CR^{a}R^{b}]ₐ—X²— (III)

wherein:
X² is selected from a direct bond or —O—;
a is 1 or 2;
R^{a} and R^{b} are both hydrogen;
R⁷ and R⁸ are independently selected from hydrogen, (1-4C)alkyl, (3-4C)cycloalkyl, (3-4C)cycloalkyl(1-2C)alkyl, or a group of sub-formula IVA:

R⁹R¹⁰N—[CR^{a}R^{b}]_{b}— (IVA)

wherein:
b is 2 or 3;
R^{a} and R^{b} are as defined above;
R⁹ and R¹⁰ are independently selected from hydrogen, (1-2C)alkyl, or
R⁹ and R¹⁰ are linked so that, together with the nitrogen atom to which they are attached, they form a 4-, 5-, or 6-membered heterocyclic ring, wherein the nitrogen atom to which R⁹ and R¹⁰ are attached is the only heteroatom present in the ring;
or R⁷ and R⁸ are linked so that, together with the nitrogen atom to which they are attached, they form a 4-, 5-, or 6-membered heterocyclic ring, wherein said heterocyclic ring optionally comprises, in addition to the nitrogen atom to which R⁷ and R⁸ are attached, an additional nitrogen atom, and the heterocyclic ring is optionally substituted by (1-4C)alkyl or —[CH₂]_{f}—NR¹³R¹⁴ (wherein f is 0, and R¹³ and R¹⁴ are independently selected from hydrogen, or (1-2C)alkyl);
(15) R^{1b} is hydrogen or a group of sub-formula III:

R⁷R⁸N—[CR^{a}R^{b}]ₐ—X²— (III)

wherein:
X² is selected from a direct bond;
a is 1;
R^{a} and R^{b} are both hydrogen;
R⁷ and R⁸ are independently selected from hydrogen, (1-4C)alkyl, (3-4C)cycloalkyl, (3-4C)cycloalkyl(1-2C)alkyl, or R⁷ and R⁹ are linked so that, together with the nitrogen atom to which they are attached, they form a 4-, 5-, or 6-membered heterocyclic ring, wherein said heterocyclic ring optionally comprises, in addition to the nitrogen atom to which R⁷ and R⁸ are attached, an additional nitrogen atom, and the heterocyclic ring is optionally substituted by (1-4C)alkyl;
(16) R^{1b} is selected from hydrogen, (methylamino)methyl, (ethylamino)methyl, (propylamino)methyl, (isopropylamino)methyl, (butylamino)methyl, (cyclobutylamino)methyl, (2-methylpropylamino)methyl, [(cyclopropylmethyl)amino]methyl, [(2-pyrrolidin-1-ylethyl)amino]methyl, azetidin-1-ylmethyl, pyrrolidin-1-ylmethyl, piperidinylmethyl, piperazinylmethyl, [3-(dimethylamino)pyrrolidin-1-yl]methyl, (4-methylpiperazin-1-yl)methyl, (4-ethylpiperazin-1-yl)methyl, or (4-isopropylpiperazin-1-yl)methyl;
(17) R^{1b} is selected from hydrogen, [(cyclopropylmethyl)amino]methyl, azetidin-1-ylmethyl, (4-ethylpiperazin-1-yl)methyl, or (4-isopropylpiperazin-1-yl)methyl;
(18) R^{1c} is selected from hydrogen, amino, (1-3C)alkyl, N-(1-3C)alkylamino, and N,N-di-[(1-3C)alkyl]amino;
(19) R^{1c} is hydrogen;
(20) m is 0;
(21) n is 0;
(22) R⁴ is amino;
(23) W is methyl;
(24) R^{1b} is a group of sub-formula (III) where at least one of R⁷ or R⁸ is (2-3C)alkenyl or (2-3C)alkynyl;
(25) R^{1b} is a group of sub-formula (M) where a least one group R⁷ or R⁸ is a group of sub-formula (IV) where X³ is a direct bond;
(26) R^{1b} is a group of sub-formula (M) where a least one group R⁷ or R⁸ is a group of sub-formula (IV) where X³ is a —(CO)—;
(27) R^{1b} is a group of sub-formula (III) where a least one group R⁷ or R⁸ is a group of sub-formula (IV) where b is 2;
28) R^{1b} is a group of sub-formula (M) where at least one group R⁷ or R⁸ is a (1-6C)alkoxy(1-6C)alkyl;
(29) R^{1b} is a group of sub-formula (III) where at least one group R⁷ or R⁸ is (1-4C)alkoxy(1-4C)alkyl, wherein the alkyl or alkoxy moiety is optionally branched;
(30) R^{1b} a group of sub-formula (III) where at least one group R⁷ or R⁸ is (1-4C)alkoxy(1-4C)alkyl selected from methoxyethyl, methoxypropyl, ethoxyethyl, ethoxypropyl, isopropoxypropyl, or 2-methoxy-2,2 dimethylethyl.
(31) R^{1b} is a group of sub-formula (III) where R⁷ and R⁸ are linked so that, together with the nitrogen atom to which they are attached, they form a 4-, 5-, 6- or 7-membered heterocyclic ring, said heterocyclic ring optionally comprising, in addition to the nitrogen atom to which R⁷ and R⁸ are attached, one or two further heteroatoms selected from N, O or S, and wherein said heterocyclic ring is optionally substituted by hydroxy, halo, (1-4C)alkyl, carbamoyl, oxo, —[CH₂]_{f}—NR¹³R¹⁴ (wherein f is 0, 1 or 2, and R¹³ and R¹⁴ are independently selected from hydrogen, (1-6C)alkyl, (3-6C)cycloalkyl or (3-6C)cycloalkyl(1-6C)alkyl), (2-4C)alkenyl, (2-4C)alkynyl, (1-4C)alkoxy, (1-4C)alkoxy(1-4C)alkyl, (1-4C)alkyl-S(O)_{q}— (where q is 0, 1 or 2), or a 5- or 6-membered heterocyclic ring comprising one to three heteroatoms selected from N, O or S provided that at least one of R¹³ or R¹⁴ are selected from (2-4C)alkenyl, (2-4C)alkynyl, (1-4C)alkoxy, (1-4C)alkoxy(1-4C)alkyl, (1-4C)alkyl-S(O)_{q}— (where q is 0, 1 or 2), or a 5- or 6-membered heterocyclic ring comprising one to three heteroatoms selected from N, O or S;
(32) R^{1b} is a group of sub-formula (III) where R⁷ and R⁸ are linked so that, together with the nitrogen atom to which they are attached, they form a 4-, 5-, 6- or 7-membered heterocyclic ring, said heterocyclic ring optionally comprising, in addition to the nitrogen atom to which R⁷ and R⁸ are attached, one or two further nitrogen atoms, and wherein said heterocyclic ring is optionally substituted as described above.
(33) R^{1b} is a group of sub-formula (M) where R⁷ and R⁸ are linked so that, together with the nitrogen atom to which they are attached, they form an azetidinyl, pyrrolidinyl, piperidinyl, piperazinyl, which may be optionally substituted as defined above;
(34) R^{1b} is a group of sub-formula (III) where R⁷ and R⁸ are linked so that, together with the nitrogen atom to which they are attached, they form a 4-, 5-, 6- or 7-membered heterocyclic ring, said heterocyclic ring being optionally substituted by (1-4C)alkyl (2-4C)alkynyl, (1-4C)alkoxy(1-4C)alkyl, (1-4C)alkyl-S(O)₂—, a 5- or 6-membered heterocyclic ring comprising one to three heteroatoms selected from N, O or S, or a group —NR¹³R¹⁴ (wherein R¹³ and R¹⁴ are independently selected from (1-6C)alkyl);
(35) R^{1b} is a group of sub-formula (III) where R⁷ and R⁸ are linked so that, together with the nitrogen atom to which they are attached, they form a 4-, 5-, 6- or 7-membered heterocyclic ring, said heterocyclic ring being optionally substituted by a 5- or 6-membered heterocyclic ring comprising one to three heteroatoms selected from N, O or S, and in particular, one or two nitrogen atoms, for example, a pyrrolidinyl, piperidinyl, piperazinyl, pyrrolyl, pyrazinyl or pyridyl group, and preferably a pyrazinyl group;
(36) R^{1b} is a group of sub-formula (III) where R^{a} and R^{b} are both hydrogen;
(37) R^{1b} is a group of sub-formula (III), where at least one of R⁷ or R⁹ is a group of formula (IV) where R^{a} and R^{b} are all hydrogen;
(38) R^{1b} is selected from hydrogen, (1-4C)alkyl, halo(1-4C)alkyl, hydroxy(1-4C)alkyl, (3-4C)cycloalkyl, (3-4C)cycloalkyl(1-2C)alkyl, (1-4C)alkoxy, (1-4C)alkoxy(1-4C)alkyl, N-(1-4C)alkylsulphamoyl, N,N-di-[(1-4C)alkyl]sulphamoyl; or

a group of sub-formula III:

R⁷R⁸N—[CR^{a}R^{b}]ₐ—X²— (III)
wherein:
   X² is selected from a direct bond, or —O—;
   a is 0, 1, 2 or 3;
   each R^{a} and R^{b} group present is independently selected from hydrogen or (1-2C)alkyl;
   R⁷ and R⁸ are independently selected from hydrogen, (1-4C)alkyl, (3-4C)cycloalkyl, (3-4C)cycloalkyl(1-2C)alkyl, (1-4C)alkoxy(1-4C)alkyl, or a group of sub-formula IV:

R⁹R¹⁰N—[CR^{a}R^{b}]_{b}X³— (IV)
wherein:
   b is 1, 2 or 3;
   R^{a} and R^{b} are as defined above;
   R⁹ and R¹⁰ are independently selected from hydrogen, (1-4C)alkyl, (3-4C)cycloalkyl, (3-4C)cycloalkyl(1-2C)alkyl, (1-4C)alkoxy(1-4C)alkyl, or R⁹ and R¹⁰ are linked so that, together with the nitrogen atom to which they are attached, they form a 4-, 5-, or 6-membered heterocyclic ring, wherein said heterocyclic ring optionally comprises, in addition to the nitrogen atom to which R⁹ and R¹⁰ are attached, an additional nitrogen atom, and the heterocyclic ring is optionally substituted by hydroxy, (1-2C)alkyl, oxo, or —[CH₂]ₑ—NR¹¹R¹² (wherein e is 0 or 1, and R¹¹ and R¹² are independently selected from hydrogen, or (1-4C)alkyl);
   X³ is a direct bond or —(C(O)—;
   or R⁷ and R⁸ are linked so that, together with the nitrogen atom to which they are attached, they form a 4-, 5-, or 6-membered heterocyclic ring, wherein said heterocyclic ring optionally comprises, in addition to the nitrogen atom to which R⁷ and R⁸ are attached, an additional nitrogen atom, and the heterocyclic ring is optionally substituted by hydroxy, (1-4C)alkyl, oxo, or
   —[CH₂]_{f}—NR¹³R¹⁴ (wherein f is 0 or 1, and R¹³ and R¹⁴ are independently selected from hydrogen, or (1-4C)alkyl);
(39) R^{1b} is selected from hydrogen, (1-2C)alkyl, halo(1-2C)alkyl, hydroxy(1-2C)alkyl, (3-4C)cycloalkyl, (3-4C)cycloalkyl(1-2C)alkyl, (1-2C)alkoxy, (1-2C)alkoxy(1-2C)alkyl, N-(1-2C)alkylsulphamoyl, N,N-di-[(1-2C)alkyl]sulphamoyl; or

a group of sub-formula III:

R⁷R⁸N—[CR^{a}R^{b}]ₐ—X²— (III)
wherein:
   X² is selected from a direct bond, or —O—;
   a is 0, 1, or 2;
   each R^{a} and R^{b} group present is independently selected from hydrogen;
   R⁷ and R⁸ are independently selected from hydrogen, (1-4C)alkyl, (3-4C)cycloalkyl, (3-4C)cycloalkyl(1-2C)alkyl, (1-2C)alkoxy(1-2C)alkyl, or a group of sub-formula IVA:

R⁹R¹⁰N—[CR^{a}R^{b}]_{b}—X³— (IVA)
wherein:
   b is 2 or 3;
   R^{a} and R^{b} are as defined above;
   X³ is a direct bond or —(C(O)—;
   R⁹ and R¹⁰ are independently selected from hydrogen, (1-2C)alkyl, (3-4C)cycloalkyl, (3-4C)cycloalkyl(1-2C)alkyl, (1-2C)alkoxy(1-2C)alkyl, or R⁹ and R¹⁰ are linked so that, together with the nitrogen atom to which they are attached, they form a 4-, 5-, or 6-membered heterocyclic ring, wherein said heterocyclic ring optionally comprises, in addition to the nitrogen atom to which R⁹ and R¹⁰ are attached, an additional nitrogen atom, and the heterocyclic ring is optionally substituted by hydroxy, (1-2C)alkyl, oxo, or —[CH₂]ₑ—NR¹¹R¹² (wherein e is 0 or 1, and R¹¹ and R¹² are independently selected from hydrogen, or (1-2C)alkyl);
   or R⁷ and R⁸ are linked so that, together with the nitrogen atom to which they are attached, they form a 4-, 5-, or 6-membered heterocyclic ring, wherein said heterocyclic ring optionally comprises, in addition to the nitrogen atom to which R⁷ and R⁸ are attached, an additional nitrogen atom, and the heterocyclic ring is optionally substituted by hydroxy, (1-4C)alkyl, oxo, or
   —[CH₂]_{f}—NR¹³R¹⁴ (wherein f is 0 or 1, and R¹³ and R¹⁴ are independently selected from hydrogen, or (1-2C)alkyl);
(40) R^{1b} is selected from hydrogen, (1-2C)alkyl, or hydroxy(1-2C)alkyl; or a group of sub-formula III:

R⁷R⁸N—[CR^{a}R^{b}]ₐ—X²— (III)
wherein:
   X² is selected from a direct bond or —O—;
   a is 1 or 2;
   each R^{a} and R^{b} group present is hydrogen;
   R⁷ and R⁸ are independently selected from hydrogen, (1-4C)alkyl, (3-4C)cycloalkyl, (3-4C)cycloalkyl(1-2C)alkyl, or a group of sub-formula IV:

R⁹R¹⁰N—[CR^{a}R^{b}]_{b}X³— (IV)
wherein:
   b is 2 or 3;
   R^{a} and R^{b} are as defined above;
   X³ is a direct bond or —(C(O)—;
   R⁹ and R¹⁰ are independently selected from hydrogen, (1-2C)alkyl, or
   R⁹ and R¹⁰ are linked so that, together with the nitrogen atom to which they are attached, they form a 4-, 5-, or 6-membered heterocyclic ring,
   wherein the nitrogen atom to which R⁹ and R¹⁰ are attached is the only heteroatom present in the ring;
   or R⁷ and R⁵ are linked so that, together with the nitrogen atom to which they are attached, they form a 4-, 5-, or 6-membered heterocyclic ring, wherein said heterocyclic ring optionally comprises, in addition to the nitrogen atom to which R⁷ and R⁸ are attached, an additional nitrogen atom, and the heterocyclic ring is optionally substituted by (1-4C)alkyl or —[CH₂]_{f}—NR¹³R¹⁴ (wherein f is 0, and R¹³ and R¹⁴ are independently selected from hydrogen, or (1-2C)alkyl);
(41) R^{1b} is selected from hydrogen, (1-4C)alkyl, hydroxy(1-4C)alkyl,

or a group of sub-formula III:

R⁷R⁸N—[CR^{a}R^{b}]ₐ—X²— (III)
wherein:
   X² is a direct bond or —O—;
   a is 1 or 2;
   R^{a} and R^{b} are both hydrogen;
   R⁷ and R⁸ are independently selected from hydrogen, (1-4C)alkyl, (3-4C)cycloalkyl, (3-4C)cycloalkyl(1-2C)alkyl, or a group of formula IV:

R⁹R¹⁰N—[CR^{a}R^{b}]_{b}X³— (IV)
wherein:
   b is 2 or 3;
   R^{a} and R^{b} are as defined above;
   X³ is a direct bond or —(C(O)—;
   R⁹ and R¹⁰ are independently selected from hydrogen, (1-4C)alkyl, or
   R⁹ and R¹⁰ are linked so that, together with the nitrogen atom to which they are attached, they form a 4-, 5- or 6-membered heterocyclic ring, and wherein the nitrogen atom to which R⁹ and R¹⁰ are attached is the only heteroatom present in the ring;
   or R⁷ and R⁸ are linked so that, together with the nitrogen atom to which they are attached, they form a 4-, 5- or 6-membered heterocyclic ring, which is optionally substituted by (1-4C)alkyl or —[CH₂]_{f}—NR¹³R¹⁴ (wherein f is 0 or 1, and R¹³ and R¹⁴ are independently selected from hydrogen, or (1-2C)alkyl), and wherein the nitrogen atom to which R⁹ and R¹⁰ are attached is the only heteroatom present in the ring;
(42) R^{1b} is selected from hydrogen or a group of sub-formula III:

R⁷R⁸N—[CR^{a}R^{b}]ₐ—X²— (III)
wherein:
   X² is selected from a direct bond or —O—;
   a is 1 or 2;
   R^{a} and R^{b} are both hydrogen;
   R⁷ and R⁸ are independently selected from hydrogen, (1-4C)alkyl, (3-4C)cycloalkyl, (3-4C)cycloalkyl(1-2C)alkyl, or a group of sub-formula IV:

R⁹R¹⁰N—[CR^{a}R^{b}]_{b}X³— (IV)
wherein:
   b is 2 or 3;
   R^{a} and R^{b} are as defined above;
   X³ is a direct bond or —(C(O)—;
   R⁹ and R¹⁰ are independently selected from hydrogen, (1-2C)alkyl, or
   R⁹ and R¹⁰ are linked so that, together with the nitrogen atom to which they are attached, they form a 4-, 5-, or 6-membered heterocyclic ring, wherein the nitrogen atom to which R⁹ and R¹⁰ are attached is the only heteroatom present in the ring;
   or R⁷ and R⁸ are linked so that, together with the nitrogen atom to which they are attached, they form a 4-, 5-, or 6-membered heterocyclic ring, wherein said heterocyclic ring optionally comprises, in addition to the nitrogen atom to which R⁷ and R⁸ are attached, an additional nitrogen atom, and the heterocyclic ring is optionally substituted by (1-3C)alkyl or —[CH₂]_{f}—NR¹³R¹⁴ (wherein f is 0 or 1, and R¹³ and R¹⁴ are independently selected from hydrogen, or (1-2C)alkyl);
(43) R^{1b} is selected from hydrogen, or a group of sub-formula III:

R⁷R⁸N—[CR^{a}R^{b}]ₐ—X²— (III)
wherein:
   X² is a direct bond;
   a is 1;
   R^{a} and R^{b} are both hydrogen;
   R⁷ and R⁸ are independently selected from hydrogen, methyl, ethyl, propyl, isopropyl, butyl, cyclopropyl, cyclobutyl, cyclopropylmethyl, or a group of sub-formula IV:

R⁹R¹⁰N—[CR^{a}R^{b}]_{b}X³— (IV)
wherein:
   b is 1 or 2;
   R^{a} and R^{b} are as defined above;
   X³ is a direct bond or —(C(O)—;
   R⁹ and R¹⁰ are independently selected from hydrogen, methyl, ethyl, propyl or R⁹ and R¹⁰ are linked so that, together with the nitrogen atom to which they are attached, they form an azetidinyl, pyrrolidinyl, or piperidinyl ring;
   or R⁷ and R⁸ are linked so that, together with the nitrogen atom to which they are attached, they form a heterocyclic ring selected from azetidinyl, pyrrolidinyl, piperidinyl, and piperazinyl, and wherein said heterocyclic ring is optionally substituted by methyl, ethyl, propyl, isopropyl or dimethylamino;
(44) R^{1b} is hydrogen or a group of sub-formula III:

R⁷R⁸N—[CR^{a}R^{b}]ₐ—X²— (III)
wherein:
   X² is selected from a direct bond, —O— or —C(O)—;
   a is 0, 1, 2, 3 or 4;
   R^{a} and R^{b} are both hydrogen or (1-2C)alkyl;
   R⁷ and R⁸ are independently selected from hydrogen, (1-6C)alkyl, (3-6C)cycloalkyl, (3-6C)cycloalkyl(1-6C)alkyl, (1-6C)alkoxy(1-6C)alkyl, or a group of formula IV:

R⁹R¹⁰N—[CR^{a}R^{b}]_{b}X³— (IV)
wherein:
   b is 1, 2 or 3;
   R^{a} and R^{b} are as defined above;
   X³ is a direct bond or —(C(O)—;
   R⁹ and R¹⁰ are independently selected from hydrogen, (1-6C)alkyl, (3-6C)cycloalkyl, (3-6C)cycloalkyl(1-6C)alkyl, (1-6C)alkoxy(1-6C)alkyl, or R⁹ and R¹⁰ are linked so that, together with the nitrogen atom to which they are attached, they form a 4-, 5-, 6- or 7-membered heterocyclic ring, said heterocyclic ring optionally comprising, in addition to the nitrogen atom to which R⁹ and R¹⁰ are attached, one or two heteroatoms selected from N, O or S, and wherein the heterocyclic ring is optionally substituted by hydroxy, halo, (1-4C)alkyl, carbamoyl, oxo, or —[CH₂]ₑ—NR¹¹R¹² (wherein e is 0, 1 or 2, and R¹¹ and R¹² are independently selected from hydrogen, (1-6C)alkyl, (3-6C)cycloalkyl or (3-6C)cycloalkyl(1-6C)alkyl);
   or R⁷ and R⁸ are linked so that, together with the nitrogen atom to which they are attached, they form a 4-, 5-, 6- or 7-membered heterocyclic ring, said heterocyclic ring optionally comprising, in addition to the nitrogen atom to which R⁷ and R⁸ are attached, one or two heteroatoms selected from N, O or S, and wherein the heterocyclic ring is optionally substituted by hydroxy, halo, (1-4C)alkyl, carbamoyl, oxo, or —[CH₂]_{f}—NR¹³R¹⁴ (wherein f is 0, 1 or 2, and R¹³ and R¹⁴ are independently selected from hydrogen, (1-6C)alkyl, (3-6C)cycloalkyl or (3-6C)cycloalkyl(1-6C)alkyl);
(45) R^{1b} is hydrogen or a group of sub-formula III:

R⁷R⁸N—[CR^{a}R^{b}]ₐ—X²— (III)
wherein:
   X² is selected from a direct bond, or —O—;
   a is 0, 1, 2, or 3;
   R^{a} and R^{b} are both hydrogen;
   R⁷ and R⁸ are independently selected from hydrogen, (1-4C)alkyl, (3-6C)cycloalkyl, (3-6C)cycloalkyl(1-4C)alkyl, (1-4C)alkoxy(1-4C)alkyl, or a group of formula IV:

R⁹R¹⁰N—[CR^{a}R^{b}]_{b}X³— (IV)
wherein:
   b is 2 or 3;
   R^{a} and R^{b} are as defined above;
   X³ is a direct bond or —C(O)—;
   R⁹ and R¹⁰ are independently selected from hydrogen, (1-4C)alkyl, (3-6C)cycloalkyl, (3-6C)cycloalkyl(1-4C)alkyl, (1-4C)alkoxy(1-4C)alkyl, or R⁹ and R¹⁰ are linked so that, together with the nitrogen atom to which they are attached, they form a 4-, 5-, or 6-membered heterocyclic ring, said heterocyclic ring optionally comprising, in addition to the nitrogen atom to which R⁹ and R¹⁰ are attached, one or two further nitrogen atoms, and wherein the heterocyclic ring is optionally substituted by hydroxy, halo, (1-4C)alkyl, carbamoyl, oxo, or —[CH₂]ₑ—NR¹¹R¹² (wherein e is 0, 1 or 2, and R¹¹ and R¹² are independently selected from hydrogen, (1-4C)alkyl, (3-6C)cycloalkyl or (3-6C)cycloalkyl(1-2C)alkyl);
   or R⁷ and R⁸ are linked so that, together with the nitrogen atom to which they are attached, they form a 4-, 5-, or 6-membered heterocyclic ring, said heterocyclic ring optionally comprising, in addition to the nitrogen atom to which R⁷ and R⁸ are attached, one or two further nitrogen atoms, and wherein the heterocyclic ring is optionally substituted by hydroxy, halo, (1-4C)alkyl, carbamoyl, oxo, or —[CH₂]_{f}—NR¹³R¹⁴ (wherein f is 0, 1 or 2, and R¹³ and R¹⁴ are independently selected from hydrogen, (1-4C)alkyl, (3-6C)cycloalkyl or (3-6C)cycloalkyl(1-2C)alkyl);
(46) R^{1b} is hydrogen or a group of sub-formula III:

R⁷R⁸N—[CR^{a}R^{b}]ₐ—X²— (III)

wherein:
X² is selected from a direct bond or —O—;
a is 1 or 2;
R^{a} and R^{b} are both hydrogen;
R⁷ and R⁸ are independently selected from hydrogen, (1-4C)alkyl, (3-4C)cycloalkyl, (3-4C)cycloalkyl(1-2C)alkyl, or a group of sub-formula IV:

R⁹R¹⁰N—[CR^{a}R^{b}]_{b}X³— (IV)

wherein:
b is 2 or 3;
R^{a} and R^{b} are as defined above;
X³ is a direct bond or —C(O)—;
R⁹ and R¹⁰ are independently selected from hydrogen, (1-2C)alkyl, or
R⁹ and R¹⁰ are linked so that, together with the nitrogen atom to which they are attached, they form a 4-, 5-, or 6-membered heterocyclic ring, wherein the nitrogen atom to which R⁹ and R¹⁰ are attached is the only heteroatom present in the ring;
or R⁷ and R⁸ are linked so that, together with the nitrogen atom to which they are attached, they form a 4-, 5-, or 6-membered heterocyclic ring, wherein said heterocyclic ring optionally comprises, in addition to the nitrogen atom to which R⁷ and R⁸ are attached, an additional nitrogen atom, and the heterocyclic ring is optionally substituted by (1-4C)alkyl or —[CH₂]_{f}—NR¹³R¹⁴ (wherein f is 0, and R¹³ and R¹⁴ are independently selected from hydrogen, or (1-2C)alkyl);
(47) R^{1b} is selected from hydrogen, amino, (4-isopropylpiperazin-1-yl)methyl, cyclopropylmethyl)amino]methyl, 4-ethylpiperazin-1-yl)methyl, 5-(azetidin-1-ylmethyl), 3-methylpyridin-2-yl, 3-pyrrolidin-1-ylpropanoyl)amino, [2-(di-methylamino)ethyl]amino}carbonyl, [(2-pyrrolidin-1-ylethyl)amino]carbonyl, {[2-(di-ethylamino)ethyl][methyl]amino}carbonyl, {[2-(di-ethylamino)ethyl]amino}carbonyl, (4-methylpiperazin-1-yl)methyl, (2-methoxyethyl)piperazin-1-yl]methyl, (4-prop-2-yn-1-ylpiperazin-1-yl)methyl, 3-(methylsulfonyl)pyrrolidin-1-yl]methyl, (2-methoxyethyl)amino]methyl, (2-methoxy-1-methylethyl)amino]methyl, (2-ethoxyethyl)amino]methyl, (2-propoxyethyl)amino]methyl, (2-methoxy-2-methylpropyl)amino]methyl, 3-methoxypropyl)amino]methyl, 3-ethoxypropyl)amino]methyl, (3-isopropoxypropyl)amino]methyl, (methylamino)methyl, (ethylamino)methyl, (propylamino)methyl, (isopropylamino)methyl, (butylamino)methyl, (isobutylamino)methyl, (cyclobutylamino)methyl, (2-pyrrolidin-1-ylethyl)amino]methyl, (prop-2-yn-1-ylamino)methyl, (prop-2-yn-1-yl)amino]methyl, (4-pyrazin-2-ylpiperazin-1-yl)methyl, (pyrrolidin-1-ylmethyl) or (dimethylamino)pyrrolidin-1-yl]methyl;
(48) R^{1b} is selected from [2-(di-methylamino)ethyl]amino}carbonyl, [(2-pyrrolidin-1-ylethyl)amino]carbonyl, {[2-(di-ethylamino)ethyl][methyl]amino}carbonyl, or {[2-(di-ethylamino)ethyl]amino}carbonyl;
(49) R^{1b} is selected from (2-methoxyethyl)amino]methyl, (2-methoxy-1-methylethyl)amino]methyl, (2-ethoxyethyl)amino]methyl, (2-propoxyethyl)amino]methyl, (2-methoxy-2-methylpropyl)amino]methyl, 3-methoxypropyl)amino]methyl, 3-ethoxypropyl)amino]methyl or (3-isopropoxypropyl)amino]methyl.
(50) R^{1b} is selected from hydrogen, amino, (4-isopropylpiperazin-1-yl)methyl, (cyclopropylmethyl)amino]methyl, (4-ethylpiperazin-1-yl)methyl, [(2-pyrrolidin-1-ylethyl)amino]carbonyl, [2-(di-methylamino)ethyl]amino}carbonyl, [2-(di-methylamino)ethyl][methyl]amino}carbonyl, {[2-(di-ethylamino)ethyl]amino}carbonyl, pyrrolidin-1-ylmethyl, (methylamino)methyl, (ethylamino)methyl, (propylamino)methyl, (isopropylamino)methyl, (cyclopropylamino)methyl, (butylamino)methyl, (cyclobutylamino)methyl, (2-pyrrolidin-1-ylethyl)amino]methyl, [3-(dimethylamino)pyrrolidin-1-yl]methyl, (4-methylpiperazin-1-yl)methyl, [(2-methoxyethyl)piperazin-1-yl]methyl, [3-(methylsulfonyl)pyrrolidin-1-yl]methyl, (4-prop-2-yn-1-ylpiperazin-1-yl)methyl, (prop-2-yn-1-ylamino)methyl, [(prop-2-yn-1-yl)(methyl)amino]methyl, (4-pyrazin-2-ylpiperazin-1-yl)methyl, [(3-ethoxypropyl)amino]methyl, [(2-propoxyethyl)amino]methyl, [(2-methoxy-2-methylpropyl)amino]methyl, [(2-methoxyethyl)amino]methyl, [(3-ethoxypropyl)amino]methyl, [(2-ethoxyethyl)amino]methyl, [(3-isopropoxypropyl)amino]methyl, [(2-methoxy-1-methylethyl)amino]methyl, and [(3-pyrrolidin-1-ylpropanoyl)amino].

For instance, the compounds of the invention may have features defined in any of paragraphs (1)-(23) above. Alternatively, they may have one or more of the features defined in items (24)-(50) above.

Suitably, R^{1a} is hydrogen, amino or (1-3C)alkyl, particularly hydrogen.

Suitably, R^{1c} is hydrogen, amino or (1-3C)alkyl, particularly hydrogen.

Suitably, m is 0, 1, 2 or 3, particularly 0.

Suitably, R², when present, is fluoro or chloro, particularly fluoro.

Suitably, n is 0, 1, 2 or 3, particularly 0.

Suitably, R³, when present, is hydroxy, fluoro or chloro, particularly fluoro.

Suitably, R⁴ is amino.

W is methyl or ethyl, particularly methyl.

Suitably, R^{1b} is hydrogen or a group of sub-formula III, particularly a group of sub-formula III.

Suitably, when R^{1b} is a group of sub-formula III, it has any one of the definitions set out in paragraphs (4) to (9), or (12) to (15) above.

Suitably, when R^{1b} is a group of sub-formula (III), it has any one of the definitions set out in paragraphs (24) to (50) above.

Suitably, X² is selected from a direct bond or —O—, and particularly a direct bond.

Suitably, integer a is 1 or 2, particularly 1.

Suitably, R^{a} and R^{b} are both hydrogen.

When R^{1b} is a group of sub-formula (III), and at least one of R⁷ or R⁸ is a group of sub-formula (IV), integer b is suitably 1, 2 or 3, particularly 2 or 3 and most particularly 2.

Suitably, R⁷, R⁸, R⁹ and R¹⁰ are as defined in anyone of paragraphs (4) to (9), (12) to (15) or (24) to (46) above.

Preferably, R^{1b} is a group of the formula R⁷R⁸N—CH₂— (i.e. a group of sub-formula III wherein X² is a direct bond, integer a is 1, and R^{a} and R^{b} are both hydrogen), wherein R⁷ and R⁸ have any one of the definitions set out herein (and are suitably as defined in any one of paragraphs (4) to (9) or (12) to (15) above).

Preferably when R⁷ and R⁸ are not linked so as to form a ring, one of R⁷ or R⁸ is hydrogen or (1-6C)alkyl, such as methyl, and most preferably one of R⁷ or R⁸ is hydrogen.

In particular, one of R⁷ or R⁸ is hydrogen or (1-6C)alkyl, such as methyl, but in particular is hydrogen, and the other is (1-6C)alkyl, (3-6C)cycloalkyl, (3-6C)cycloalkyl(1-6C)alkyl, (1-6C)alkoxy(1-6C)alkyl, (2-3C)alkenyl, (2-3C)alkynyl, or a group of formula (IV) as defined above.

In a particular embodiment, R^{1b} is a group of sub-formula (III) where R⁷ and R⁸ are linked so that, together with the nitrogen atom to which they are attached, they form a 4-, 5-, 6- or 7-membered heterocyclic ring, said heterocyclic ring may optionally contain one or two further heteroatoms selected from N, O or S, and be optionally substituted as described above. In particular, R⁷ and R⁸ are linked so that, together with the nitrogen atom to which they are attached, they form a 4-, 5- or 6-membered heterocyclic ring which optionally contains one or two further N atoms. Particular examples for rings formed by R⁷ and R⁸ include azetidinyl, pyrrolidinyl, piperidinyl or piperazinyl, and in particular azetidinyl, pyrrolidinyl, or piperazinyl.

Suitably rings R⁷ and R⁸ and the nitrogen atom to which they are attached are substituted by one or more groups, suitably from one to three groups, and most preferably one group which are selected from those defined above. Particular examples of such substituents include (1-4C)alkyl, —[CH₂]_{f}—NR¹³R¹⁴ (wherein f is 0), and R¹³ and R¹⁴ are independently selected from (1-6C)alkyl, (2-4C)alkynyl, (1-4C)alkoxy(1-4C)alkyl, or (1-4C)alkyl-S(O)₂—. Other particular examples of substitutents include 5- or 6-membered heterocyclic rings comprising one to three heteroatoms selected from N, O or S, and in particular, 5- or 6-membered heterocyclic rings containing one or two nitrogen atoms, for example, pyrrolidinyl, piperidinyl, piperazinyl, pyrrolyl, pyrazinyl or pyridyl group. In a particular embodiment a pyrazine group is substituted onto a ring formed by R⁷ and R⁸ and the nitrogen atom to which they are attached.

Examples of Compounds of Formula (I) include compounds of formula (IA):

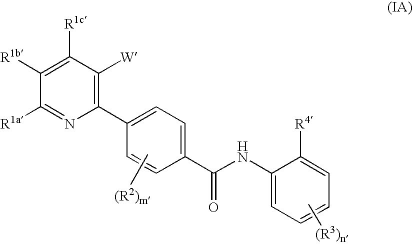

wherein:

### R^{1a} is selected from hydrogen, amino, (1-3C)alkyl, N-(1-3C)alkylamino, N,N-di-(1-3C)alkylamino, or a group of the sub-formula II:

R^{5′}R^{6′}N—X^{1′}—[CR^{a′}R^{b′}]_{q}— (II)

wherein:
q′ is 1, 2 or 3;
each R^{a′} and R^{b′} group present is independently selected from hydrogen, halo, hydroxy or (1-4C)alkyl;
X^{1′} is selected from a direct bond or —C(O)—; and
R^{5′} and R^{6′} are each independently selected from hydrogen or (1-3C)alkyl;
and wherein if R^{1a′, is a N-(}1-3C)alkylamino or N,N-di-(1-3C)alkylamino group, the (1-3C)alkyl moiety is optionally substituted by hydroxy or (1-2C)alkoxy;
   R^{1b′} is selected from:
(i) hydrogen, (1-6C)alkyl, halo(1-6C)alkyl, hydroxy(1-6C)alkyl, (3-6C)cycloalkyl, (3-6C)cycloalkyl(1-4C)alkyl, (1-6C)alkoxy, (1-6C)alkoxy(1-6C)alkyl, N-(1-4C)alkylsulphamoyl, N,N-di-[(1-4C)alkyl]sulphamoyl; or
(ii) a group of sub-formula III:

R^{7′}R^{8′}N—[CR^{a′}R^{b′}]_{a′}—X^{2′}— (III)

wherein:
X^{2′} is selected from a direct bond, —O— or —C(O)—;
a′ is 0, 1, 2, 3 or 4;
R^{a′} and R^{b′} are as defined above;
R^{7′} and R^{8′} are independently selected from hydrogen, (1-6C)alkyl, (3-6C)cycloalkyl, (3-6C)cycloalkyl(1-6C)alkyl, (1-6C)alkoxy(1-6C)alkyl, or a group of formula IV:

R^{9′}R^{10′}N—[CR^{a′}R^{b′}]_{b′}— (IV)

wherein:
b′ is 1, 2 or 3;
R^{a′} and R^{b′} are as defined above;

R^{9′} and R^{10′} are independently selected from hydrogen, (1-6C)alkyl, (3-6C)cycloalkyl, (3-6C)cycloalkyl(1-6C)alkyl, (1-6C)alkoxy(1-6C)alkyl, or R^{9′} and R^{10′} are linked so that, together with the nitrogen atom to which they are attached, they form a 4-, 5-, 6- or 7-membered heterocyclic ring, said heterocyclic ring optionally comprising, in addition to the nitrogen atom to which R^{9′} and R^{10′} are attached, one or two further heteroatoms selected from N, O or S, and wherein said heterocyclic ring is optionally substituted by hydroxy, halo, (1-4C)alkyl, carbamoyl, oxo, or —[CH₂]_{e′}—NR^{1′1}R^{12′} (wherein e′ is 0, 1 or 2, and R^{11′} and R^{12′} are independently selected from hydrogen, (1-6C)alkyl, (3-6C)cycloalkyl or (3-6C)cycloalkyl(1-6C)alkyl);

or R^{7′} and R^{8′} are linked so that, together with the nitrogen atom to which they are attached, they form a 4-, 5-, 6- or 7-membered heterocyclic ring, said heterocyclic ring optionally comprising, in addition to the nitrogen atom to which R^{7′} and R^{8′} are attached, one or two further heteroatoms selected from N, O or S, and wherein said heterocyclic ring is optionally substituted by hydroxy, halo, (1-4C)alkyl, carbamoyl, oxo, or —[CH₂]_{f}—NR^{13′}R^{14′} (wherein f′ is 0, 1 or 2, and R^{13′} and R^{14′} are independently selected from hydrogen, (1-6C)alkyl, (3-6C)cycloalkyl or (3-6C)cycloalkyl(1-6C)alkyl); or
(iii) a group of the sub-formula VII:

Q′-Z′-Y′— (VII)

wherein:
Y′ is a direct bond or —[CR^{a′}R^{b′}]_{x′}—, where integer x′ is 1 to 4 and R^{a′} and R^{b′} are as defined above;
Z′ is absent or selected from —O—, —S—, —SO—, —SO₂—, —NH—SO₂—, —SO₂—NH— or —C(O)—; and

Q′ is a carbon-linked heterocyclyl or a heterocyclyl-(1-6C)alkyl group, said heterocyclyl or a heterocyclyl-(1-6C)alkyl group being optionally substituted on the heterocyclyl ring by one or more substituent groups (for example 1, 2 or 3), which may be the same or different, selected from halo, oxo, cyano, hydroxy, trifluoromethyl, amino, carboxy, carbamoyl, mercapto, sulphamoyl, (1-3C)alkyl, (2-3C)alkenyl, (2-3C)alkynyl, (1-3C)alkoxy, (1-3C)alkanoyl, (1-3C)alkanoyloxy, (1-3C)alkoxy(1-3C)alkyl, (1-3C)alkoxycarbonyl, halo(1-3C)alkyl, N-[(1-3C)alkyl]amino, N,N-di-[(1-3C)alkyl]amino, N-[(1-3C)alkoxy(1-3C)alkyl]amino, N,N-di-[(1-3C)alkoxy(1-3C)alkyl]amino, N-[(1-3C)alkoxy(1-3C)alkyl]-N-[(1-3C)alkyl]amino, N-(1-3C)alkylcarbamoyl, N,N-di-[(1-3C)alkyl]carbamoyl, (1-3C)alkylthio, (1-3C)alkylsulphinyl, (1-3C)alkylsulphonyl, N-(1-3C)alkylsulphamoyl, N,N-di-[(1-3C)alkyl]sulphamoyl;

R^{1c′} is selected from hydrogen, halo, cyano, hydroxy, trifluoromethyl, trifluoromethoxy, amino, carboxy, carbamoyl, mercapto, sulphamoyl, (1-3C)alkyl, (2-3C)alkenyl, (2-3C)alkynyl, (1-3C)alkoxy, (1-3C)alkanoyl, (1-3C)alkanoyloxy, N-(1-3C)alkylamino, N,N-di-[(1-3C)alkyl]amino, (1-3C)alkanoylamino, N-(1-3C)alkylcarbamoyl, N,N-di-(1-3C)alkylcarbamoyl, (1-3C)alkylthio, (1-3C)alkylsulphinyl, (1-3C)alkylsulphonyl, (1-3C)alkoxycarbonyl, N-(1-3C)alkylsulphamoyl, and N,N-di-(1-3C)alkylsulphamoyl;

integer m′ is 0, 1, 2, 3 or 4;

### R^{2′} is halo;

integer n′ is 0, 1, 2, 3 or 4;
R^{3′} is selected from halo, cyano, hydroxy, trifluoromethyl, trifluoromethoxy, amino, carboxy, carbamoyl, mercapto, sulphamoyl, (1-3C)alkyl, (2-3C)alkenyl, (2-3C)alkynyl, (1-3C)alkoxy, (1-3C)alkanoyl, (1-3C)alkanoyloxy, N-(1-3C)alkylamino, N,N-di-[(1-3C)alkyl]amino, (1-3C)alkanoylamino, N-(1-3C)alkylcarbamoyl, N,N-Di(1-3C)alkylcarbamoyl, (1-3C)alkylthio, (1-3C)alkylsulphinyl, (1-3C)alkylsulphonyl, (1-3C)alkoxycarbonyl, N-(1-3C)alkylsulphamoyl, and N,N-di-(1-3C)alkylsulphamoyl;

### R^{4′} is amino or hydroxy; and

### W′ is methyl or ethyl;

or a pharmaceutically acceptable salt or pro-drug thereof.

A particular group of compounds according to the present invention have the general formula (V) shown below

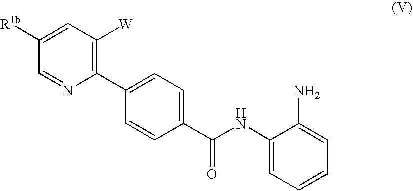

wherein W is as hereinbefore defined (and is particularly methyl) and R^{1b} has any one of the definitions set out hereinbefore (and is particularly as defined in any one of paragraphs (4) to (17) or (24) to (50) above).

A further particular sub-group of compounds of the present invention have the general formula (VI)

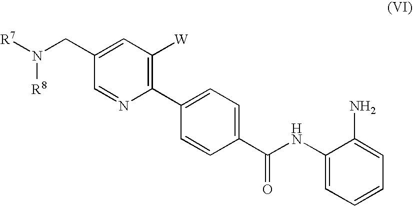

wherein W is as hereinbefore defined (and is particularly methyl) and R⁷ and R⁸ each have any one of the definitions set out hereinbefore (and are particularly as defined for the group of sub-formula III in any one of paragraphs (4) to (9) or (12) to (15) or (24) to (46) above).

Particular novel compounds of the invention include any one of the following:
N-(2-aminophenyl)-4-(3-methylpyridin-2-yl)benzamide;
N-(2-aminophenyl)-4-{5-[(4-isopropylpiperazin-1-yl)methyl]-3-methylpyridin-2-yl}benzamide;
N-(2-aminophenyl)-4-(5-{[(cyclopropylmethyl)amino]methyl}-3-methylpyridin-2-yl)benzamide;
N-(2-aminophenyl)-4-{5-[(4-ethylpiperazin-1-yl)methyl]-3-methylpyridin-2-yl}benzamide;
N-(2-aminophenyl)-4-[5-(azetidin-1-ylmethyl)-3-methylpyridin-2-yl]benzamide;
4-(5-amino-3-methylpyridin-2-yl)-N-(2-aminophenyl)benzamide;
N-(2-aminophenyl)-4-{3-methyl-5-[(3-pyrrolidin-1-ylpropanoyl)amino]pyridin-2-yl}benzamide;
6-(4-{[(2-aminophenyl)amino]carbonyl}phenyl)-N-[2-(dimethylamino)ethyl]-N,5-dimethylnicotinamide;
6-(4-{[(2-aminophenyl)amino]carbonyl}phenyl)-N-[2-(dimethylamino)ethyl]-5-methylnicotinamide;
6-(4-{[(2-aminophenyl)amino]carbonyl}phenyl)-5-methyl-N-(2-pyrrolidin-1-ylethyl)nicotinamide;
6-(4-{[(2-aminophenyl)amino]carbonyl}phenyl)-N-[2-(diethylamino)ethyl]-5-methylnicotinamide;
N-(2-aminophenyl)-4-(3-ethylpyridin-2-yl)benzamide;
N-(2-aminophenyl)-4-{3-methyl-5-[(4-methylpiperazin-1-yl)methyl]pyridin-2-yl}benzamide;
N-(2-aminophenyl)-4-(5-{[4-(2-methoxyethyl)piperazin-1-yl]methyl}-3-ethylpyridin-2-yl)benzamide;
N-(2-aminophenyl)-4-{3-methyl-5-[(4-prop-2-yn-1-ylpiperazin-1-yl)methyl]pyridin-2-yl}benzamide;
N-(2-aminophenyl)-4-(3-methyl-5-{[3-(methylsulfonyl)pyrrolidin-1-yl]methyl}pyridin-2-yl)benzamide;
N-(2-aminophenyl)-4-(5-{[(2-methoxyethyl)amino]methyl}-3-methylpyridin-2-yl)benzamide;
N-(2-aminophenyl)-4-(5-{[(2-methoxy-1-methylethyl)amino]methyl}-3-methylpyridin-2-yl)benzamide;
N-(2-aminophenyl)-4-(5-{[(2-ethoxyethyl)amino]methyl}-3-methylpyridin-2-yl)benzamide;
N-(2-aminophenyl)-4-(3-methyl-5-{[(2-propoxyethyl)amino]methyl}pyridin-2-yl)benzamide;
N-(2-aminophenyl)-4-(5-{[(2-methoxy-2-methylpropyl)amino]methyl}-3-methylpyridin-2-yl)benzamide;
N-(2-aminophenyl)-4-(5-{[(3-methoxypropyl)amino]methyl}-3-methylpyridin-2-yl)benzamide;
N-(2-aminophenyl)-4-(5-{[(3-ethoxypropyl)amino]methyl}-3-methylpyridin-2-yl)benzamide;
N-(2-aminophenyl)-4-(5-{[(3-isopropoxypropyl)amino]methyl}-3-methylpyridin-2-yl)benzamide;
N-(2-aminophenyl)-4-{3-methyl-5-[(methylamino)methyl]pyridin-2-yl}benzamide;
N-(2-aminophenyl)-4-{5-[(ethylamino)methyl]-3-methylpyridin-2-yl}benzamide;
N-(2-aminophenyl)-4-{3-methyl-5-[(propylamino)methyl]pyridin-2-yl}benzamide;
N-(2-aminophenyl)-4-{5-[(isopropylamino)methyl]-3-methylpyridin-2-yl}benzamide;
N-(2-aminophenyl)-4-{5-[(butylamino)methyl]-3-methylpyridin-2-yl}benzamide;
N-(2-aminophenyl)-4-{5-[(isobutylamino)methyl]-3-methylpyridin-2-yl}benzamide;
N-(2-aminophenyl)-4-{5-[(ethylamino)methyl]-3-methylpyridin-2-yl}benzamide;
N-(2-aminophenyl)-4-(3-methyl-5-{[(2-pyrrolidin-1-ylethyl)amino]methyl}pyridin-2-yl)benzamide;
N-(2-aminophenyl)-4-{3-methyl-5-[(prop-2-yn-1-ylamino)methyl]pyridin-2-yl}benzamide;
N-(2-aminophenyl)-4-(3-methyl-5-{[methyl(prop-2-yn-1-yl)amino]methyl}pyridin-2-yl)benzamide
N-(2-aminophenyl)-4-{3-methyl-5-[(4-pyrazin-2-ylpiperazin-1-yl)methyl]pyridin-2-yl}benzamide;
N-(2-aminophenyl)-4-[3-methyl-5-(pyrrolidin-1-ylmethyl)pyridin-2-yl]benzamide
N-(2-aminophenyl)-4-(5-{[(3S)-3-(dimethylamino)pyrrolidin-1-yl]methyl}-3-methylpyridin-2-yl)benzamide; and
N-(2-aminophenyl)-4-(5-{[(3R)-3-(dimethylamino)pyrrolidin-1-yl]methyl}-3-methylpyridin-2-yl)benzamide.

Another aspect of the present invention provides a process for preparing a compound of formula (I) or a pharmaceutically acceptable salt or pro-drug form thereof (wherein R^{1a}, R^{1b}, R^{1c}, R², R³, R⁴, integer m, integer n and W are, unless otherwise specified, as hereinbefore defined), said process comprising the steps of:

(a) the reaction of a compound of the formula (A)

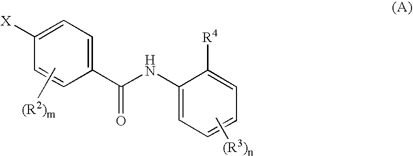

wherein X is a reactive group, with a compound of the formula (B)

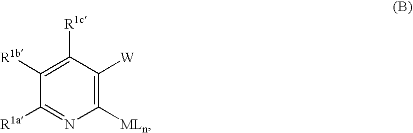

wherein
R^{1a′} is a group R^{1a} as hereinbefore defined or a precursor thereof,
R^{1b′} is a group R^{1b} as hereinbefore defined or a precursor thereof,
R^{1c′} is a group R^{1c} as hereinbefore defined or a precursor thereof,
M is a metal,
L is a ligand, and
integer n′ is 0 to 3;

and wherein if any one of said groups R^{1a′}, R^{1b′} or R^{1c′} is a precursor for a R^{1a}, R^{1b} or R^{1c} group respectively, then said process thereafter comprises a step of converting the compound formed by the reaction of a compound of the formula (A) with a compound of the formula (B) to a compound of formula (I) (by converting the precursor of any one of groups R^{1a}, R^{1b} or R^{1c} group to the appropriate R^{1a}, R^{1b} or R^{1c} group); or

(b) The reaction of a compound of the formula (C)

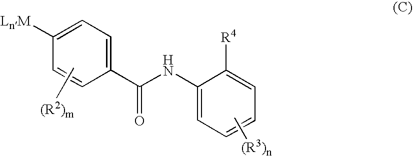

wherein M, L and integer n′ are as defined above, with a compound of the formula (D)

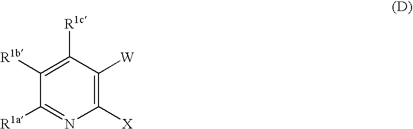

wherein R^{1a′}, R^{1b′} and R^{1c′} are as defined above and X is a reactive group; and wherein if any one of said groups R^{1a′}, R^{1b′} or R^{1c′} is a precursor for a R^{1a}, R^{1b} or R^{1c} group respectively, then said process comprises an additional step thereafter of converting the compound formed by the reaction of a compound of the formula (C) with a compound of the formula (D) to a compound of formula (I) (by converting the precursor of any one of groups R^{1a}, R^{1b} or R^{1c} group to the appropriate R^{1a}, R^{1b} or R^{1c} group); or

(c) the reaction to couple an acid to an amine, in particular in the presence of a suitable amide coupling agent, in particular 4-(4,6-dimethoxy-1,3,5-triazinyl-2-yl)-4-methylmorpholinium chloride, of a compound of the formula (E)

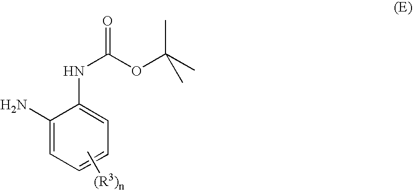

with a compound of the formula (F)

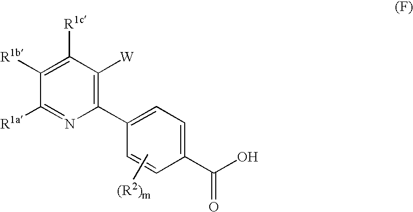

wherein R^{1a′}, R^{1b′} and R^{1c′} are as defined above, and wherein if any one of said groups R^{1a′}, R^{1b′} or R^{1c′} is a precursor for a R^{1a}, R^{1b} or R^{1c} group respectively, then said process comprises an additional step thereafter of converting the compound formed by the reaction of a compound of the formula (E) with a compound of the formula (F) to a compound of formula (I) (by converting the precursor of any one of groups R^{1a}, R^{1b} or R^{1c} group to the appropriate R^{1a}, R^{1b} or R^{1c} group); or

and thereafter if necessary:
i) converting a compound of the formula (I) into another compound of the formula (I); and/or
ii) removing any protecting groups.

A suitable base for process (a), (b) or (c) is, for example, an organic amine base such as, for example, pyridine, 2,6-lutidine, collidine, 4-dimethylaminopyridine, triethylamine, morpholine, N-methylmorpholine or diazabicyclo[5.4.0]undec-7-ene, or, for example, an alkali or alkaline earth metal carbonate or hydroxide, for example sodium carbonate, potassium carbonate, calcium carbonate, sodium hydroxide or potassium hydroxide, or, for example, an alkali metal hydride, for example sodium hydride, or an alkaline metal hydrogencarbonate such as sodium hydrogencarbonate, or a metal alkoxide such as sodium ethoxide.

A suitable reactive group X is, for example, a halo or a sulphonyloxy group, for example a chloro, bromo, iodo, methanesulphonyloxy, trifluoromethanesulphonyloxy or toluene-4-sulphonyloxy group.

The reactions are conveniently carried out in the presence of a suitable inert solvent or diluent, for example an alkanol or ester such as methanol, ethanol, isopropanol or ethyl acetate, a halogenated solvent such as methylene chloride, chloroform or carbon tetrachloride, an ether such as tetrahydrofuran, 1,2-dimethoxyethane or 1,4-dioxan, an aromatic solvent such as toluene, or a dipolar aprotic solvent such as N N-dimethylformamide, N,N-dimethylacetamide, N-methylpyrrolidin-2-one or dimethylsulphoxide. The reactions are conveniently carried out at a temperature in the range, for example, 10 to 250° C., preferably in the range 40 to 80° C.;

Metal M may be any metal that is known in the literature to form organometallic compounds that undergo catalytic cross coupling reactions. Examples of suitable metals include boron, tin, zinc, magnesium.

A suitable value for n′ is dependent on the metal M, but is usually in the range 0-3.

Suitable values for the ligand L, when present, include, for example, a hydroxy, a halo, (1-4C)alkoxy or (1-6C)alkyl ligand, for example a hydroxy, bromo, chloro, fluoro, iodo, methoxy, ethoxy, propoxy, isopropoxy, butoxy, methyl, ethyl, propyl, isopropyl or butyl ligand or, where n is 2 and M is boron, the two ligands present may be linked such that, together with the boron atom to which they are attached, they form a ring.

Suitably, the group ML_{n′} is a group of the formula —BL¹L², where B is boron and L¹ and L² are as defined for ligand L above. In particular, the ligands L¹ and L² may be linked such that, together with the boron atom to which they are attached, they form a ring. For example, L¹ and L² together may define an oxy-(2-4C)alkylene-oxy group, for example an oxyethyleneoxy, pinacolato (—O—C(CH₃)₂C(CH₃)₂—O—) or oxypropyleneoxy group such that, together with the boron atom to which they are attached, they form a cyclic boronic acid ester group.

A suitable catalyst for process (a) or (b) includes, for example, a metallic catalyst such as a palladium(0), palladium(II), nickel(0) or nickel(II) catalyst, for example tetrakis(triphenylphosphine)palladium(0), palladium(II) chloride, palladium(II) bromide, bis(triphenylphosphine)palladium(II) chloride, tetrakis(triphenylphosphine)nickel(0), nickel(II) chloride, nickel(II) bromide, bis(triphenylphosphine)nickel(II) chloride or dichloro[1-1′-bis(diphenylphosphino)ferrocene]palladium(II). In addition, a free radical initiator may conveniently be added, for example an azo compound such as azo(bisisobutyronitrile).

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, reductive amination of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halo group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green and P. G. M. Wuts, Protective Groups in Organic Synthesis, John Wiley and Sons, 1999). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

Where any one of groups R^{1a′}, R^{1b′} or R^{1c′} is a precursor for a R^{1a}, R^{1b} or R^{1c} group respectively, it may converted into a compound of formula I by converting the precursor of any one of groups R^{1a}, R^{1b} or R^{1c} to the appropriate R^{1a}, R^{1b} or R^{1c} group using standard chemical techniques that are well known to those skilled in the art. Examples of possible R^{1a′}, R^{1b′} or R^{1c′} precursor groups (particularly R^{1b′} precursor groups) include nitro, amino, hydroxy or alcohol-containing groups (e.g. —CH₂OH), aldehyde-containing groups (e.g. —CHO), carboxylic acid-containing groups (e.g. —(CH₂)₀₋₃—COOH), ester containing groups (e.g. —(CH₂)₀₋₃—COOR^{Z}, where R^{Z} is (1-4C)alkyl), activated ester containing groups (e.g. —(CH₂)₀₋₃—COOR^{y}, where R^{y} is a group such as, for example, pentafluorophenyl), amide containing groups (e.g. —CONH₂), acyl halide containing groups (e.g —(CH₂)₀₋₃—C(O)X, where X is a halogen such as for example chloride) or a group —CH₂—X where X is a reactive group as hereinbefore defined. For instance, possible R^{1a′}, R^{1b′} or R^{1c′} precursor groups (particularly R^{1b′} precursor groups) are hydroxy or alcohol-containing groups (e.g. —CH₂OH), aldehyde-containing groups (e.g. —CHO), carboxylic acid-containing groups (e.g. —(CH₂)₀₋₃—COOH), ester containing groups (e.g. —(CH₂)₀₋₃—COOR^{Z}, where R^{Z} is (1-4C)alkyl), amide containing groups (e.g. —CONH₂), a group —CH₂—X where X is a reactive group as hereinbefore defined, or an activated ester group, such as a pentafluorphenoxy ester or an acyl chloride. A person skilled in the art will appreciate how to select the most appropriate precursor group for conversion into the desired R^{1a}, R^{1b} or R^{1c} substituent groups.

For example, a compound of the present invention having the formula (VI) shown below (i.e. a compound of formula I in which R^{1b} is a group of sub-formula III wherein X² is a direct bond, integer a is 1 and R^{a} and R^{b} are both hydrogen; integer m is 0; W is (1-2C)alkyl, particularly methyl and R⁴ is amino)

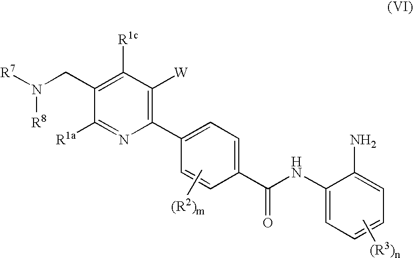

is suitably prepared by a process (process (d)) comprising the reaction, in the presence of a suitable base, of a compound of formula (G), wherein the aniline may be protected and R^{1b′} is a precursor for the R⁷R⁸N—CH₂— group in the compound of formula (VI) above, said precursor having the formula —CH₂—X, wherein X is a reactive group as hereinbefore defined,

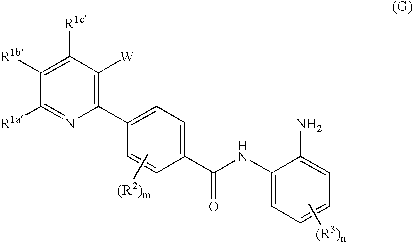

with a compound of formula (H);

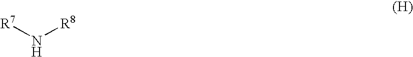

and thereafter, if necessary, removing any protecting groups and/or converting any precursor groups R^{1a′} or R^{1c′} to groups R^{1a} and R^{1c} respectively.

In a particular embodiment, R^{1a′}, R^{1c′}, R^{1a} and R^{1b} are all hydrogen.

Alternatively, a compound of general structural formula (VI) above may be prepared by a process (process (e)) which comprises the reaction, in the presence of a suitable reducing agent and a suitable acid, of a compound of formula (G), wherein the aniline may be protected and R^{1b′} is a precursor for the R⁷R⁸N—CH₂— group in the compound of formula (VI) above, said precursor having the formula —CHO (formyl):

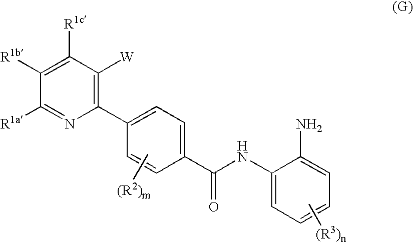

with a compound of formula (H);

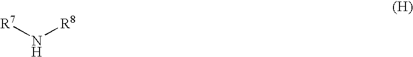

and thereafter, if necessary, removing any protecting groups and/or converting any precursor groups R^{1a′} and R^{1c′} to groups R^{1a} and R^{1c} respectively. As before, groups R^{1a′}, R^{1c′}, R^{1a} and R^{1c} are suitably hydrogen.

A suitable base for process (d) is, for example, an organic amine base such as, for example, pyridine, 2,6-lutidine, collidine, 4-dimethylaminopyridine, triethylamine, morpholine, N-methylmorpholine or diazabicyclo[5.4.0]undec-7-ene, or, for example, an alkali or alkaline earth metal carbonate or hydroxide, for example sodium carbonate, potassium carbonate, calcium carbonate, sodium hydroxide or potassium hydroxide, or, for example, an alkali metal hydride, for example sodium hydride, or an alkaline earth metal hydrogencarbonate such as sodium hydrogencarbonate, or a metal alkoxide such as sodium ethoxide.

A suitable reducing agent for process (e) includes, for example, an inorganic borohydride salt such as, sodium borohydride, sodium triacetoxyborohydride or sodium cyanoborohydride.

A suitable acid for process (e), includes a Bronsted acid such as, for example formic acid, acetic acid, trifluoroacetic acid, hydrochloric acid, sulphuric acid, paratoluene sulfonic acid or camphor sulfonic acid; or a Lewis acid of formula MX_{z}, wherein M is a metal, X is a reactive group as hereindefined and z is in the range of 1-6 and the value of z will depend on the metal M. Typical examples of suitable Lewis acids include boron trifluoride, scandium(III) trifluoromethanesulfonate, tin(VI) chloride, titanium(IV) isopropoxide or zinc(II) chloride.

Another aspect of the present invention provides a particular process (process (f)) for preparing the intermediate compound (G) above wherein R^{1b′} is —CHO (formyl), said process comprising the steps of:

i) the reaction of a compound of the formula (J), wherein L and integer n′ are as hereinbefore defined,

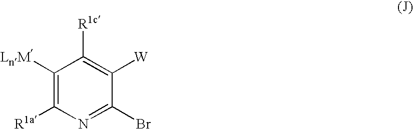

with a suitable formulating agent, to produce a compound of the formula (K).

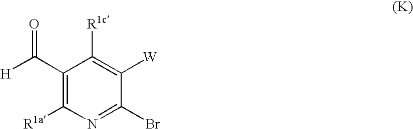

ii) the reaction, in the presence of a suitable base, of a compound the formula (K), made in step (i) above, with a compound of formula (IV)

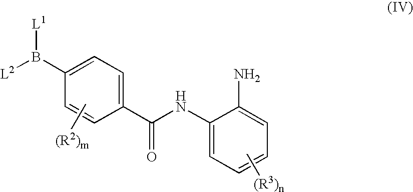

wherein L¹ and L² are ligands as hereinbefore defined; and thereafter, if necessary, removing any protecting groups.

Metal M′ may be any metal known in the literature to form nucleophilic organometallic compounds. Examples of suitable metals include lithium and magnesium.

A suitable value for n′ and L are as hereinbefore defined.

A suitable choice of formulating agent for process (f) (step (i)) includes, for example, carbon monoxide, N-formylmorpholine, N-formylpiperidine and N,N dimethylformamide.

Suitable values for the ligands L¹ and L², which are present on the boron atom, are as hereinbefore defined.

A suitable choice of base and catalyst for process (f) (step (ii)) are as hereinbefore defined for processes (a) and (b).

In an additional example, a compound of the present invention having the formula (VII) shown below i.e. a compound of formula I in which R^{1b} is a group of sub-formula III wherein X² is —C(O), integer a is 0; integer m is 0; integer n is 0; W is (1-2C)alkyl, particularly methyl, and R⁴ is amino,

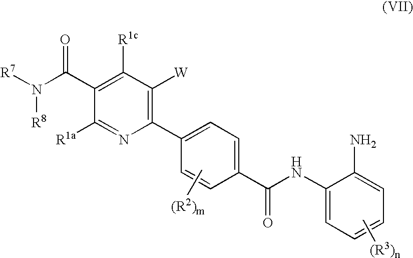

is suitably prepared by a process (process (g)) comprising the reaction, in the presence of a suitable base, of a compound of formula (G), wherein the aniline may be protected and R^{1b′} is a precursor for the R⁷R⁸N—C(O)— group in the compound of formula (VI) above, said precursor suitably having the formula —COOH, or an activated form thereof, such as an acyl chloride or an active ester

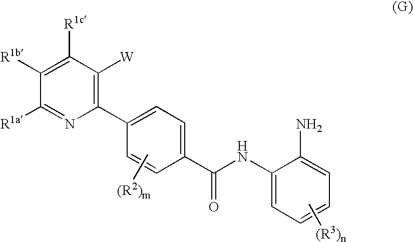

with a compound of formula (H);

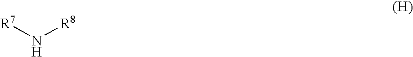

and thereafter, if necessary, removing any protecting groups and/or converting any precursor groups R^{1a′} or R^{1c′} to groups to groups R^{1a} and R^{1c} respectively. Suitably, R^{1a}, R^{1c}, R^{1a′} and R^{1c′} are all hydrogen.

A further aspect of the present invention provides a particular process (process (h)) for preparing the intermediate compound (G) above wherein R^{1b′} is —COOH (carboxy), said process comprising the steps of:

i) the reaction of a compound of the formula (J), wherein M, L and integer n are as hereinbefore defined,

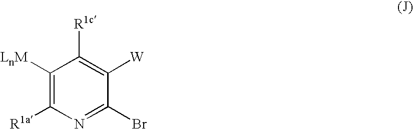

with a suitable electrophilic reagent, to produce a compound of the formula (L).

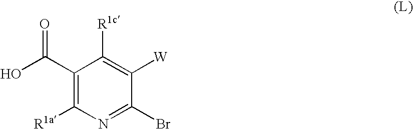

ii) the reaction, in the presence of a suitable base and suitable catalyst, of a compound the formula (L), made in step (i) above, with a compound of formula (IV)

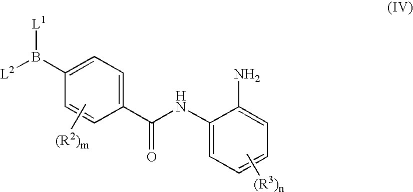

wherein L¹ and L² are ligands as hereinbefore defined;

and thereafter, if necessary, removing any protecting groups.

Metal M may be any metal known in the literature to form nucleophilic organometallic compounds. Examples of suitable metals include lithium and magnesium.

A suitable value for n and L are as hereinbefore defined.

A suitable choice of electrophilic reagent for process (h) (step (i)) includes, for example, carbon dioxide.

Suitable values for the ligands L¹ and L², which are present on the boron atom, are as hereinbefore defined.

A suitable choice of base and catalyst for process (f) (step (ii)) are as hereinbefore defined for processes (a) and (b).

In a further example, a compound of the present invention having the formula (VIII) shown below (i.e. a compound of formula I in which R^{1b} is a group of sub-formula III wherein X² is 0, integer a is 0, R⁷ is hydrogen and R⁸ is a group of substructure IV, wherein X³ is —C(O)—, integer b is 2 and R^{a} and R^{b} are all hydrogen, integer m is 0, integer n is 0, and R⁴ is amino)

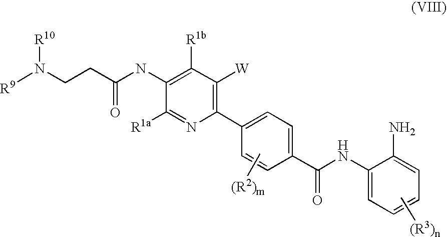

is suitably prepared by a process (process (i)) comprising the reaction, in the presence of a suitable base, of a compound of formula (M), wherein the aniline may be protected and X is a reactive group as hereinbefore defined

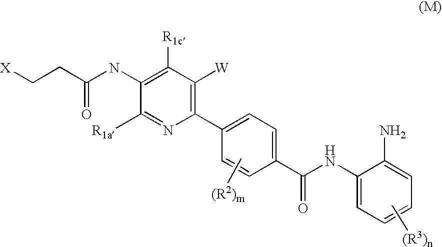

with a compound of formula (N)

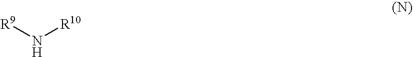

and thereafter, if necessary, removing any protecting groups and/or converting any precursor groups R^{1a′} and R^{1c′} to groups R^{1a} and R^{1c} respectively. Again, R^{1a′}, R^{1b′}, R^{1a} and R^{1b} may all be hydrogen.

A suitable choice of base for process (i) is as hereinbefore defined for process (d).

Another aspect of the present invention provides a particular process (process (O)) for preparing an intermediate of compound (M) above, said process comprising the steps of:

i) the reaction, in the presence of a suitable base and suitable catalyst of a compound of formula (IV), wherein L¹ and L² are ligands as hereinbefore defined;

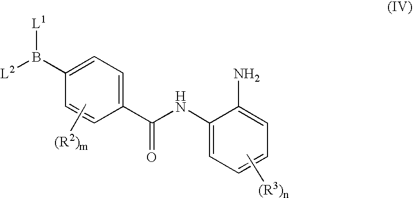

with a compound of formula (O)

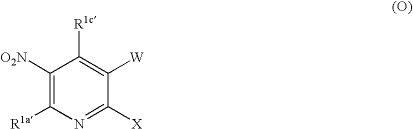

to produce a compound (P).

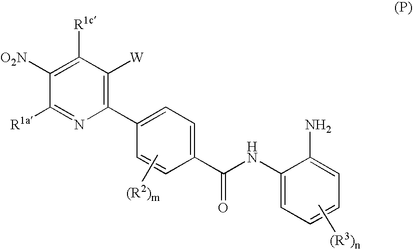

ii) the reaction of compound (P) made in step (i) above in the presence of a suitable reducing agent or under suitable reducing conditions, to produce a compound of formula (Q)

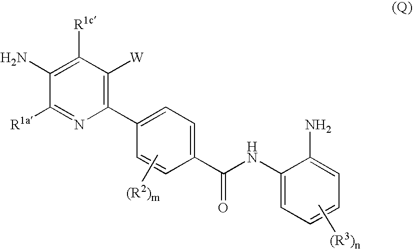

iii) the reaction of a compound of formula (Q) made in step (ii) above, in the presence of a suitable base with a compound of formula (R), wherein X is a reactive group as hereinbefore defined and Y is a group known in the chemical art to form amides, for example, groups such as chloro, bromo or pentafluorophenoxy.

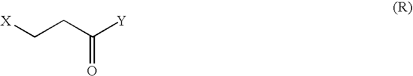

and thereafter if necessary removing any protecting groups.

A suitable choice of base and catalyst for process (j) (step (i)) are as hereinbefore defined for processes (a) and (b).

A suitable choice of reducing agent or reducing conditions for process (j) (step (ii)), include for example, the use of hydrogen in the presence of a palladium catalyst, such as, for example palladium (II) hydroxide; the use of metallic iron in the presence of acetic acid and cyclohexene in the presence of a suitable palladium catalyst.

A suitable base for process (j) (step (iii)) is as hereinbefore defined for process (d).

A further aspect of the present invention provides a particular process (process (k)) for preparing an intermediate of compound (F) above wherein W is specifically —C₂H₅ (ethyl), said process comprising the steps of:

i) the reaction of a compound of formula (S), wherein X is a reactive group as hereinbefore defined,

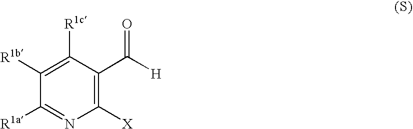

with a compound of formula (T), wherein as outlined above the carboxylate group maybe suitably protected and M, L & integer n′ are as hereinbefore defined,

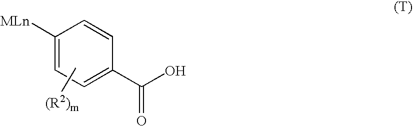

to produce the compound (U)

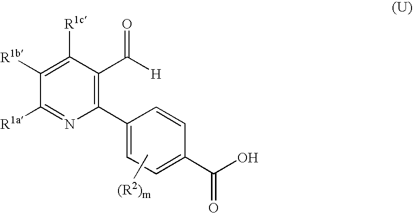

ii) The conversion of a compound of formula (U) into a compound of formula (F) wherein W is C₂H₅— (ethyl), using techniques well known to one skilled in the chemical art.
An example of such a conversion includes, for example, the reaction of a compound (U), in the presence of a suitable base, for example sodium tert-butoxide, with methyltriphenyl phosphonium iodide, followed by hydrogenation over a catalyst such as palladium-on-carbon.

A suitable choice of base and catalyst for process (k) (step (i)) are as hereinbefore defined for processes (a) and (b).

Metal M may be any metal that is known in the literature to form organometallic compounds that undergo catalytic cross coupling reactions, in particular boron.

A suitable value for n′ is dependent on the metal M, but is usually in the range 0-3.

Suitable values for the ligand L are as defined above.

It will be appreciated that it may be necessary/desirable to protect the carboxylate group. A suitable protecting group for this group is, for example, an esterifying group as hereinbefore defined, such as for example a methyl ester (—CO2Me).

### Biological Assays

The following assays can be used to measure the effects of the compounds of the present invention as HDAC inhibitors, as inhibitors in vitro of recombinant human HDAC1 produced in Hi5 insect cells, and as inducers in vitro & in vivo of Histone H3 acetylation in whole cells and tumours. They also assess the ability of such compounds to inhibit proliferation of human tumour cells.

(a) In Vitro Enzyme Assay of Recombinant HDAC1

HDAC inhibitors were screened against recombinant human HDAC1 produced in Hi5 insect cells. The enzyme was cloned with a FLAG tag at the C-terminal of the gene and affinity purified using Anti-FLAG M2 agarose from SIGMA (A2220).

The deacetylase assays were carried out in a 50 μl reaction. HDAC1 (75 ng of enzyme) diluted in 15 μl of reaction buffer (25 mM Tris HCl (pH 8), 137 mM NaCl, 2.7 mM KCl, 1 mM MgCl₂) was mixed with either buffer alone (10 μl) or buffer containing compound (10 μl) for 30 minutes at ambient temperature. 25 μM acetylated histone H4 peptide (KI 174 Biomol) diluted in 25 μl of buffer was then added to the reaction and incubated for one hour at ambient temperature. The reaction was stopped by addition of an equal volume (50 μl) Fluor de Lys developer (Biomol) containing Trichostatin A at 2 μM. The reaction was allowed to develop for 30 minutes at ambient temperature and then fluorescence measured at an excitation wavelength of 360 nM and an emission wavelength of 465 nM. IC₅₀ values for HDAC enzyme inhibitors were determined by performing dose response curves with individual compounds and determining the concentration of inhibitor producing fifty percent decrease in the maximal signal (diluent control).

(b) In Vitro Assay of Inhibition of Proliferation in Whole Cells

Inhibition of proliferation in whole cells was assayed using the Promega cell titer 96 aqueous proliferation assay (Promega #G5421). HCT116 cells were seeded in 96 well plates at 1×10³ cells/well, and allowed to adhere overnight. They were treated with inhibitors for 72 hours. 20 μl of the tetrazolium dye MTS was added to each well and the plates were reincubated for 3 hours. Absorbance was then measured on a 96 well plate reader at 490 nM. The IC₅₀ values for HDAC inhibitors were determined by performing dose response curves with individual compounds and determining the concentration of inhibitor producing fifty percent decrease in maximal signal (diluent control).

(c) In Vitro Enzyme Assay of Histone Deacetylase Activity in Whole Cells

Histone H3 acetylation in whole cells was measured using immunohistochemistry and analysis using the Cellomics arrayscan. A549 or HCT116 cells were seeded in 96 well plates at 1×10⁴ cells/well, and allowed to adhere overnight. They were treated with inhibitors for 24 hours and then fixed in 1.8% formaldehyde in tris buffered saline (TBS) for one hour. Cells were permeabilized with ice-cold methanol for 5 minutes, rinsed in TBS and then blocked in TBS 3% low-fat dried milk for 90 minutes. Cells were then incubated with polyclonal antibodies specific for the acetylated histone H3 (Upstate #06-599) diluted 1 in 500 in TBS 3% milk for one hour. Cells were rinsed three times in TBS and then incubated with fluorescein conjugated secondary antibodies (Molecular Probes #A11008) & Hoechst 333542 (1 μg/ml) (Molecular Probes #H3570) in TBS plus 1% Bovine serum albumin (Sigma #B6917) for one hour. Unbound antibody was removed by three rinses with TBS and after the final rinse 100 μl of TBS was added to the cells and the plates sealed and analysed using the Cellomics arrayscan.

EC₅₀ values for HDAC inhibitors were determined by performing dose response curves with individual compounds and then determining the concentration of inhibitor producing fifty percent of the maximal signal (reference compound control—Trichostatin A (Sigma)).

Although the pharmacological properties of the compounds of the formula (I) vary with structural change as expected, in general activity possessed by compounds of the Formula I, may be demonstrated at the following concentrations or doses in one or more of the above tests (a)-(b):—

Test (a:—IC₅₀ in the range, for example, <0.060 μM;

Test (b):—IC₅₀ in the range, for example, <0.75 μM;

Test (c):—IC₅₀ in the range, for example, <0.75 μM.

The following table discloses various biological data for a representative selection of compounds of the present invention. Comparative test data is also provided for N-(2-aminophenyl)-4-pyridin-2-ylbenzamide (Comparator).

| Example | Test (a) IC₅₀ (μM) | Test (b) IC₅₀ (μM) |
|---|---|---|
| 2 | 0.019 | 0.238 |
| 4 | 0.021 | 0.31 |
| Comparator | 0.089 | 2.33 |

According to a further aspect of the invention there is provided a pharmaceutical composition which comprises a compound of the formula (I), or a pharmaceutically acceptable salt or pro-drug thereof, as defined hereinbefore in association with a pharmaceutically-acceptable diluent or carrier.

The composition may be in a form suitable for oral administration, for example as a tablet or capsule, for parenteral injection (including intravenous, subcutaneous, intramuscular, intravascular or infusion) as a sterile solution, suspension or emulsion, for topical administration as an ointment or cream or for rectal administration as a suppository.

In general the above compositions may be prepared in a conventional manner using conventional excipients.

The compound of formula (I) will normally be administered to a warm-blooded animal at a unit dose within the range 5-5000 mg/m² body area of the animal, i.e. approximately 0.1-100 mg/kg, and this normally provides a therapeutically-effective dose. A unit dose form such as a tablet or capsule will usually contain, for example 1-250 mg of active ingredient. Preferably a daily dose in the range of 1-50 mg/kg is employed. However the daily dose will necessarily be varied depending upon the host treated, the particular route of administration, and the severity of the illness being treated. Accordingly the optimum dosage may be determined by the practitioner who is treating any particular patient.

We have found that the compounds defined in the present invention, or a pharmaceutically acceptable salt thereof, are effective cell cycle inhibitors (anti-cell proliferation agents), which property is believed to arise from their HDAC inhibitory properties. We also believe that the compounds of the present invention may be involved in the inhibition of angiogenesis, activation of apoptosis and differentiation. Accordingly the compounds of the present invention are expected to be useful in the treatment of diseases or medical conditions mediated alone or in part by HDAC enzymes, i.e. the compounds may be used to produce a HDAC inhibitory effect in a warm-blooded animal in need of such treatment. Thus, the compounds of the present invention provide a method for treating the proliferation of malignant cells characterised by inhibition of HDAC enzymes, i.e. the compounds may be used to produce an anti-proliferative effect mediated alone or in part by the inhibition of HDACs.

According to one aspect of the present invention there is provided a compound of the formula (I), or a pharmaceutically acceptable salt or pro-drug thereof, as defined hereinbefore for use in a method of treatment of the human or animal body by therapy.

Thus according to a further aspect of the invention there is provided a compound of the formula (I), or a pharmaceutically acceptable salt or pro-drug thereof, as defined hereinbefore for use as a medicament.

According to a further aspect of the invention there is provided the use of a compound of the formula (I), or a pharmaceutically acceptable salt or pro-drug thereof, as defined hereinbefore in the manufacture of a medicament for use in the production of a HDAC inhibitory effect in a warm-blooded animal such as man.

According to a further feature of this aspect of the invention there is provided a method for producing a HDAC inhibitory effect in a warm-blooded animal, such as man, in need of such treatment which comprises administering to said animal an effective amount of a compound of the formula (I), or a pharmaceutically acceptable salt or pro-drug thereof, as defined hereinbefore.

According to a further aspect of the invention there is provided the use of a compound of the formula (I), or a pharmaceutically acceptable salt or pro-drug thereof, as defined hereinbefore in the manufacture of a medicament for use in the production of a cell cycle inhibitory (anti-cell-proliferation) effect in a warm-blooded animal such as man.

According to a further feature of this aspect of the invention there is provided a method for producing a cell cycle inhibitory (anti-cell-proliferation) effect in a warm-blooded animal, such as man, in need of such treatment which comprises administering to said animal an effective amount of a compound of the formula (I), or a pharmaceutically acceptable salt or pro-drug thereof, as defined hereinbefore.

According to an additional feature of this aspect of the invention there is provided a method of treating cancer in a warm-blooded animal, such as man, in need of such treatment which comprises administering to said animal an effective amount of a compound of the formula (I), or a pharmaceutically acceptable salt or pro-drug thereof, as defined hereinbefore.

According to a further feature of the invention there is provided a compound of the formula (I), or a pharmaceutically acceptable salt or pro-drug thereof, as defined hereinbefore in the manufacture of a medicament for use in the treatment of cancer.

According to an additional feature of this aspect of the invention there is provided a compound of the formula (I), or a pharmaceutically acceptable salt or pro-drug thereof, or a pharmaceutically acceptable salt thereof, as defined hereinbefore, for use in the treatment of cancer.

In a further aspect of the present invention there is provided the use of a compound of the formula (I), or a pharmaceutically acceptable salt or pro-drug thereof, as defined hereinbefore, in the manufacture of a medicament for use in lung cancer, colorectal cancer, breast cancer, prostate cancer, lymphoma and/or leukaemia.

In a further aspect of the present invention there is provided a method of treating lung cancer, colorectal cancer, breast cancer, prostate cancer, lymphoma or leukaemia, in a warm-blooded animal, such as man, in need of such treatment which comprises administering to said animal an effective amount of a compound of the formula (I), or a pharmaceutically acceptable salt or pro-drug thereof, as defined hereinbefore.

Cancers that are amenable to treatment with the present invention include oesophageal cancer, myeloma, hepatocellular, pancreatic and cervical cancer, Ewings tumour, neuroblastoma, kaposis sarcoma, ovarian cancer, breast cancer, colorectal cancer, prostate cancer, bladder cancer, melanoma, lung cancer [including non small cell lung cancer (NSCLC) and small cell lung cancer (SCLC)], gastric cancer, head and neck cancer, brain cancer, renal cancer, lymphoma and leukaemia.

There is further provided is a compound of the formula (I), or a pharmaceutically acceptable salt or pro-drug thereof, as defined hereinbefore, for use in a method of treating inflammatory diseases, autoimmune diseases and allergic/atopic diseases.

In particular a compound of the formula (I), or a pharmaceutically acceptable salt thereof, as defined hereinbefore, is provided for use in a method of treating inflammation of the joint (especially rheumatoid arthritis, osteoarthritis and gout), inflammation of the gastro-intestinal tract (especially inflammatory bowel disease, ulcerative colitis and gastritis), inflammation of the skin (especially psoriasis, eczema, dermatitis), multiple sclerosis, atherosclerosis, spondyloarthropathies (ankylosing spondylitis, psoriatic arthritis, arthritis connected to ulcerative colitis), AIDS-related neuropathies, systemic lupus erythematosus, asthma, chronic obstructive lung diseases, bronchitis, pleuritis, adult respiratory distress syndrome, sepsis, and acute and chronic hepatitis (either viral, bacterial or toxic).

Further provided is a compound of the formula (I), or a pharmaceutically acceptable salt or pro-drug thereof, as defined hereinbefore, for use as a medicament in the treatment of inflammatory diseases, autoimmune diseases and allergic/atopic diseases in a warm-blooded animal such as man.

In particular a compound of the formula (I), or a pharmaceutically acceptable salt or pro-drug thereof, as defined hereinbefore, is provided for use as a medicament in the treatment of inflammation of the joint (especially rheumatoid arthritis, osteoarthritis and gout), inflammation of the gastro-intestinal tract (especially inflammatory bowel disease, ulcerative colitis and gastritis), inflammation of the skin (especially psoriasis, eczema, dermatitis), multiple sclerosis, atherosclerosis, spondyloarthropathies (ankylosing spondylitis, psoriatic arthritis, arthritis connected to ulcerative colitis), AIDS-related neuropathies, systemic lupus erythematosus, asthma, chronic obstructive lung diseases, bronchitis, pleuritis, adult respiratory distress syndrome, sepsis, and acute and chronic hepatitis (either viral, bacterial or toxic).

Further provided is the use of a compound of the formula (I), or a pharmaceutically acceptable salt thereof, as defined hereinbefore, in the manufacture of a medicament for use in the treatment of inflammatory diseases, autoimmune diseases and allergic/atopic diseases in a warm-blooded animal such as man.

As stated above the size of the dose required for the therapeutic or prophylactic treatment of a particular cell-proliferation disease will necessarily be varied depending on the host treated, the route of administration and the severity of the illness being treated. A unit dose in the range, for example, 1-100 mg/kg, preferably 1-50 mg/kg is envisaged.

The HDAC inhibitory activity defined hereinbefore may be applied as a sole therapy or may involve, in addition to a compound of the invention, one or more other substances and/or treatments. Such conjoint treatment may be achieved by way of the simultaneous, sequential or separate administration of the individual components of the treatment. In the field of medical oncology it is normal practice to use a combination of different forms of treatment to treat each patient with cancer. In medical oncology the other component(s) of such conjoint treatment in addition to the cell cycle inhibitory treatment defined hereinbefore may be: surgery, radiotherapy or chemotherapy. Such chemotherapy may include one or more of the following categories of anti-tumour agents:

(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegafur, raltitrexed, methotrexate, cytosine arabinoside and hydroxyurea; antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecin);
(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) Agents which inhibit cancer cell invasion (for example metalloproteinase inhibitors like marimastat and inhibitors of urokinase plasminogen activator receptor function);
(iv) inhibitors of growth factor function, for example such inhibitors include growth factor antibodies, growth factor receptor antibodies (for example the anti-erbb2 antibody trastuzumab [Herceptin™] and the anti-erbb1 antibody cetuximab [C225]), farnesyl transferase inhibitors, MEK inhibitors, tyrosine kinase inhibitors and serine/threonine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, AZD 1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI-1033)), for example inhibitors of the platelet-derived growth factor family and for example inhibitors of the hepatocyte growth factor family;
(v) antiangiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin);
(vi) vascular damaging agents such as Combretastatin A4 and compounds disclosed in International Patent Applications WO 99/02166, WO00/40529, WO 00/41669, WO01/92224, WO02/04434 and WO02/08213;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy;
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies;
(x) Cell cycle inhibitors including for example CDK inhibitors (eg flavopiridol) and other inhibitors of cell cycle checkpoints (eg checkpoint kinase); inhibitors of aurora kinase and other kinases involved in mitosis and cytokinesis regulation (eg mitotic kinesins); and other histone deacetylase inhibitors; and
(xi) differentiation agents (for example retinoic acid and vitamin D).

According to this aspect of the invention there is provided a pharmaceutical composition comprising a compound of the formula (I) as defined hereinbefore and an additional anti-tumour substance as defined hereinbefore for the conjoint treatment of cancer.

In addition to their use in therapeutic medicine, the compounds of formula (I) and their pharmaceutically acceptable salts thereof, are also useful as pharmacological tools in the development and standardisation of in vitro and in vivo test systems for the evaluation of the effects of inhibitors of cell cycle activity in laboratory animals such as cats, dogs, rabbits, monkeys, rats and mice, as part of the search for new therapeutic agents.

The invention will now be illustrated in the following Examples in which, generally:

(i) operations were carried out at ambient temperature, i.e. in the range 17 to 25° C. and under an atmosphere of an inert gas such as argon unless otherwise stated;
(ii) evaporations were carried out by rotary evaporation in vacuo and work-up procedures were carried out after removal of residual solids by filtration;
(iii) column chromatography (by the flash procedure) and medium pressure liquid chromatography (MPLC) were performed on Merck Kieselgel silica (Art. 9385) or Merck Lichroprep RP-18 (Art. 9303) reversed-phase silica obtained from E. Merck, Darmstadt, Germany or using proprietary pre-packed normal phase silica cartridges, for example Redisep™ disposable chromatography cartridges obtained from Presearch Ltd., Hitchin, UK, or high pressure liquid chromatography (HPLC) was performed on C18 reverse phase silica, for example on a Dynamax C-18 60 Å preparative reversed-phase column;
(iv) yields, where present, are not necessarily the maximum attainable;
(v) in general, the structures of the end-products of the Formula (I) were confirmed by nuclear magnetic resonance (NMR) and/or mass spectral techniques; fast-atom bombardment (FAB) mass spectral data were obtained using a Platform spectrometer and, where appropriate, either positive ion data or negative ion data were collected; NMR chemical shift values were measured on the delta scale [proton magnetic resonance spectra were determined using a Jeol JNM EX 400 spectrometer operating at a field strength of 400 MHz, Varian Gemini 2000 spectrometer operating at a field strength of 300 MHz or a Bruker AM300 spectrometer operating at a field strength of 300 MHz—measurements were taken at ambient temperature unless otherwise specified;
(vi) intermediates were not generally fully characterised and purity was assessed by thin layer chromatographic, HPLC, infra-red (IR) and/or NMR analysis;
(vii) melting points are uncorrected and were determined using a Mettler SP62 automatic melting point apparatus or an oil-bath apparatus; melting points for the end-products of the formula (I) were determined after crystallisation from a conventional organic solvent such as ethanol, methanol, acetone, ether or hexane, alone or in admixture;
(viii) the following abbreviation has been used:—

DMSO dimethylsulphoxide

BOC t-butoxycarbonyl

### EXAMPLE 1

### Preparation of N-(2-aminophenyl)-4-(3-methylpyridin-2-yl)benzamide

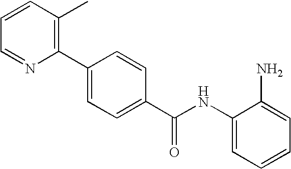

N-(2-t-Butoxycarbonylaminophenyl)-4-(4,4,5,5-tetramethyl-1,3,2-dioxaborolan-2-yl)benzamide (400 mg, 0.913 mmol—prepared as described in Method 13, page 60, of International patent publication number WO 03/087057), 2-chloro-3-methylpyridine (120 mg, 0.913 mmol), tetrakis(triphenylphosphine)palladium (50 mg, 0.043 mmol), 1,2-dimethoxyethane (4 ml) and a saturated aqueous solution of sodium hydrogen carbonate (2 ml) were stirred at 120° C. under an atmosphere of nitrogen in a microwave for 45 minutes. The mixture was allowed to cool before being partitioned between dichloromethane and water. The organics were separated, dried over sodium sulfate, filtered and evaporated. The crude product was purified by chromatography eluting with ethyl acetate then rechromatographed eluting with 1-3% methanol in dichloromethane to afford the title compound as a white solid (90 mg, 33%); NMR Spectrum: (DMSO-d₆) 2.35 (s, 3H) 4.90 (s, 2H), 6.60 (m, 1H), 6.78 (m, 1H), 6.97 (m, 1H), 7.19 (d, 1H), 7.32 (m, 1H), 7.66 (d, 2H), 7.74 (m, 1H), 8.07 (d, 2H), 8.50 (d, 1H), 9.70 (s, 1H); Mass Spectrum: M+H⁺304.

### EXAMPLE 2

### Preparation of N-(2-aminophenyl)-4-{5-[(4-isopropylpiperazin-1-yl)methyl]-3-methylpyridin-2-yl}benzamide

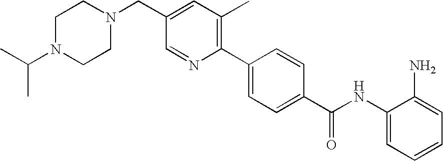

t-Butyl (2-{[4-(5-formyl-3-methylpyridin-2-yl)benzoyl]amino}phenyl)carbamate (1 g, 2.32 mmol, see Method 3 below) was dissolved in tetrahydrofuran (50 ml) and then 1-isopropylpiperazine (445 mg, 3.48 mmol) and acetic acid (134 μl, 2.32 mmol) added. The solution was stirred at ambient temperature for 2 hours then sodium triacetoxyborohydride (738 mg, 3.48 mmol) added and the mixture stirred for a further 18 hours. The solvents were evaporated under reduced pressure and the residue partitioned between dichloromethane (50 ml) and saturated aqueous sodium bicarbonate solution (50 ml). The dichloromethane layer was washed with brine (50 ml), dried over magnesium sulfate, filtered and evaporated under reduced pressure. The residue was purified by flash column chromatography eluting with 50% ethyl acetate in isohexane then a gradient of 10% to 15% methanol in dichloromethane. Fractions containing the t-butoxycarbonyl (BOC) protected product were evaporated and re-dissolved in dichloromethane (5 ml), trifluoroacetic acid (5 ml) added and the mixture stirred at ambient temperature for 2 hours. The mixture was absorbed onto an SCX-2 column and washed with methanol (2 column volumes) then the product eluted with a 2M solution of ammonia in methanol (2 column volumes) and concentrated under reduced pressure, to give the title compound (357 mg, 35%); NMR Spectrum: (DMSO-d₆) 0.97 (d, 6H), 2.37 (s, 3H), 2.44 (br m, 8H), 2.62 (m, 1H), 3.52 (s, 2H), 4.92 (br s, 2H), 6.62 (m, 1H), 6.80 (d, 1H), 6.99 (m, 1H), 7.21 (d, 1H), 7.66 (s, 1H), 7.69 (d, 2H), 8.07 (d, 2H), 8.42 (s, 1H), 9.72 (s, 1H); Mass Spectrum: M+H⁺444.

### EXAMPLE 3

### Preparation of N-(2-aminophenyl)-4-{5-[(4-ethylpiperazin-1-yl)methyl]-3-methylpyridin-2-yl}benzamide

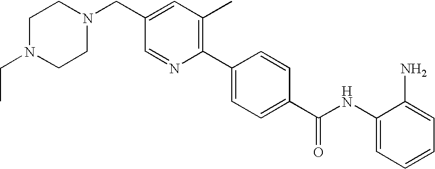

t-Butyl (2-{[4-(5-formyl-3-methylpyridin-2-yl)benzoyl]amino}phenyl)carbamate (1 g, 2.32 mmol, see Method 3 below) was dissolved in tetrahydrofuran (50 ml) then 1-ethylpiperazine (397 mg, 3.48 mmol) and acetic acid (134 μl, 2.32 mmol) added. The solution was stirred at ambient temperature for 2 hours then sodium triacetoxyborohydride (738 mg, 3.48 mmol) added and the mixture stirred for a further 18 hours. The solvents were evaporated under reduced pressure and the residue partitioned between dichloromethane (50 ml) and saturated aqueous sodium bicarbonate solution (50 ml). The dichloromethane layer was washed with brine (50 ml), dried over magnesium sulfate, filtered and then evaporated under reduced pressure. The residue was purified by flash column chromatography eluting with 50% ethyl acetate in isohexane then a gradient of 10% to 15% methanol in dichloromethane. Fractions containing the t-butoxycarbonyl (BOC) protected product were evaporated and re-dissolved in dichloromethane (5 ml), trifluoroacetic acid (5 ml) added and stirred at ambient temperature for 2 hours. The mixture was absorbed onto an SCX-2 column and washed with methanol (2 column volumes) then the product eluted with a 2M solution of ammonia in methanol (2 column volumes) and concentrated under reduced pressure, to give the title compound (319 mg, 32%); NMR Spectrum: (DMSO-d₆) 0.99 (t, 3H), 2.32 (q, 2H), 2.41 (br m, 8H), 2.37 (s, 3H), 3.52 (s, 2H), 4.92 (br s, 2H), 6.62 (m, 1H), 6.80 (d, 1H), 6.99 (m, 1H), 7.21 (d, 1H), 7.66 (s, 1H), 7.69 (d, 2H), 8.07 (d, 2H), 8.42 (s, 1H), 9.72 (s, 1H); Mass Spectrum: M+H⁺430.

### EXAMPLE 4

### Preparation of N-(2-aminophenyl)-4-[5-(azetidin-1-ylmethyl)-3-methylpyridin-2-yl]benzamide

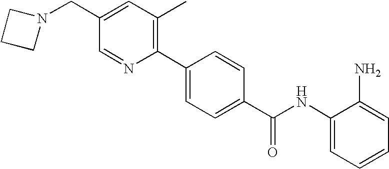

t-Butyl (2-{[4-(5-formyl-3-methylpyridin-2-yl)benzoyl]amino}phenyl)carbamate (1 g, 2.32 mmol, see Method 3 below) was dissolved in tetrahydrofuran (50 ml) and then azetidine (199 mg, 3.48 mmol) and acetic acid (134 μl, 2.32 mmol) added. The solution was stirred at ambient temperature for 2 hours then sodium triacetoxyborohydride (738 mg, 3.48 mmol) added and the mixture stirred for a further 18 hours. The solvents were evaporated under reduced pressure and the residue partitioned between dichloromethane (50 ml) and saturated aqueous sodium bicarbonate solution (50 ml). The dichloromethane layer was washed with brine (50 ml), dried over magnesium sulfate, filtered and evaporated under reduced pressure. The residue was purified by flash column chromatography eluting with 50% ethyl acetate in isohexane then a gradient of 10% to 15% methanol in dichloromethane. Fractions containing the t-butoxycarbonyl (BOC) protected product were evaporated and re-dissolved in dichloromethane (5 ml), trifluoroacetic acid (5 ml) added and stirred at ambient temperature for 2 hours. The mixture was absorbed onto an SCX-2 column and washed with methanol (2 column volumes) then the product eluted with a 2M solution of ammonia in methanol (2 column volumes) and concentrated under reduced pressure, to give the title compound (274 mg, 32%); NMR Spectrum: (DMSO-d₆) 2.01 (m, 2H), 2.36 (s, 3H), 3.18 (t, 4H), 3.58 (s, 2H), 4.92 (br s, 2H), 6.62 (m, 1H), 6.80 (d, 1H), 6.99 (m, 1H), 7.21 (d, 1H), 7.63 (s, 1H), 7.68 (d, 2H), 8.07 (d, 2H), 8.40 (s, 1H), 9.72 (s, 1H); Mass Spectrum: M+H⁺373.

### EXAMPLE 5

### Preparation of N-(2-aminophenyl)-4-(5-{[(cyclopropylmethyl)amino]methyl}-3-methylpyridin-2-yl)-benzamide

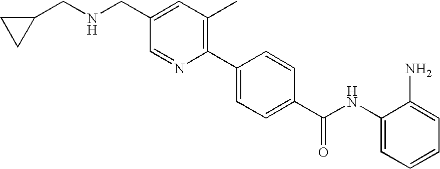

### Method A

t-Butyl (2-{[4-(5-formyl-3-methylpyridin-2-yl)benzoyl]amino}phenyl)carbamate (1 g, 2.32 mmol, see Method 3 below) was dissolved in dichloromethane (35 ml) then aminomethylcyclopropane (302 μl, 3.48 mmol) and titanium isopropoxide (1.39 ml, 4.64 mmol) added. The solution was stirred at ambient temperature for 1 hour then sodium borohydride (351 mg, 9.28 mmol) in methanol (6 ml) added and left stirring for a further 18 hours. Saturated aqueous sodium bicarbonate solution (30 ml) was added to the reaction mixture and stirred vigorously for 10 minutes. The mixture was filtered, the dichloromethane layer separated, washed with brine (30 ml) then dried over magnesium sulfate, filtered and evaporated under reduced pressure. The residue was purified by flash column chromatography eluting with 50% ethyl acetate in isohexane then 15% methanol in dichloromethane. Fractions containing the t-butoxycarbonyl (BOC) protected product were evaporated then re-dissolved in dichloromethane (5 ml), trifluoroacetic acid (5 ml) added and stirred at ambient temperature for 2 hours before being absorbed onto an SCX-2 column. The column was washed with methanol (2 column volumes) and the product eluted with a 2M solution of ammonia in methanol (2 column volumes) which was concentrated under reduced pressure, to give the title compound (158 mg, 18%); NMR Spectrum: (DMSO-d₆) 0.10 (m, 2H), 0.40 (m, 2H), 0.91 (m, 1H), 2.25 (br s, 1H), 2.35 (s, 3H), 2.41 (d, 2H), 3.76 (s, 2H), 4.90 (br s, 2H), 6.61 (m, 1H), 6.79 (d, 1H), 6.97 (m, 1H), 7.20 (d, 1H), 7.66 (d, 2H), 7.68 (s, 1H), 8.05 (d, 2H), 8.44 (s, 1H), 9.70 (s, 1H); Mass Spectrum: M+H⁺387.

### Method B

t-Butyl (2-{[4-(5-{[(cyclopropylmethyl)amino]methyl}-3-methylpyridin-2-yl)benzoyl]amino}phenyl)carbamate (878 mg, 1.80 mmol, see Method 1 below) was stirred and suspended in dichloromethane (8 ml). Trifluoroacetic acid (2 ml) was added and the solution stirred for 3 hours at ambient temperature. The solution was absorbed onto an SCX-2 column and the column washed with methanol (3 column volumes) and the product eluted with a 2M solution of ammonia in methanol (3 column volumes). The ammonia/methanol solution was concentrated under reduced pressure to give the title compound (586 mg, 84%); NMR Spectrum: (DMSO-d₆) 0.11 (m, 2H), 0.40 (m, 2H), 0.91 (m, 1H), 2.35 (s, 3H), 2.41 (d, 2H), 3.76 (s, 2H), 4.90 (br s, 2H), 6.61 (m, 1H), 6.79 (d, 1H), 6.97 (m, 1H), 7.20 (d, 1H), 7.66 (d, 2H), 7.69 (s, 1H), 8.06 (d, 2H), 8.44 (s, 1H), 9.71 (s, 1H); Mass Spectrum: M+H⁺387.

### EXAMPLE 6

### Preparation of (4-(5-amino-3-methylpyridin-2-yl)-N-(2-aminophenyl)benzamide)

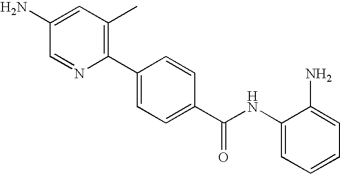

tert-Butyl (2-{[4-(5-amino-3-methylpyridin-2-yl)benzoyl]amino}phenyl)carbamate (120 mg, 0.29 mmol, see Method 6 below) was dissolved in dichloromethane (2 ml), trifluoroacetic acid (2 ml) added and the solution stirred at ambient temperature for 2 hours. The solution was absorbed onto an SCX-2 column and washed with methanol (2 column volumes) then the product eluted with a 2M solution of ammonia in methanol (2 column volumes) and concentrated under reduced pressure, to give the title compound (41 mg, 45%); NMR Spectrum: (DMSO-d₆) 2.27 (s, 3H), 4.90 (br s, 2H), 5.38 (br s, 2H), 6.62 (m, 1H), 6.80 (m, 1H), 6.86 (s, 1H), 6.98 (m, 1H), 7.20 (m, 1H), 7.61 (d, 2H), 7.91 (s, 1H), 8.01 (d, 2H), 9.66 (s, 1H); Mass Spectrum: M+H⁺319.

### EXAMPLE 7

### Preparation of (N-(2-aminophenyl-4-[3-methyl-5-[(3-pyrrolidin-1-ylpropanoyl)amino]pyridin-2-yl]benzamide)

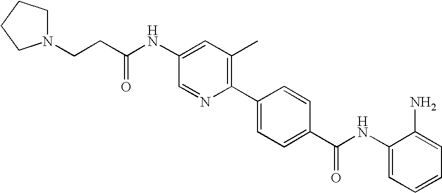

tert-Butyl {2-[(4-{5-[(3-bromopropanoyl)amino]-3-methylpyridin-2-yl}benzoyl)amino]phenyl}carbamate (189 mg, 0.34 mmol, see Method 5 below) was dissolved in tetrahydrofuran (5 ml), pyrrolidine (421 μl, 5.13 mmol) was added and the solution stirred at ambient temperature for 72 hours. The solvents were evaporated under reduced pressure and the residue partitioned between dichloromethane (5 ml) and water (5 ml). The dichloromethane layer was filtered through a PTFE cup and evaporated under reduced pressure. The residue was re-dissolved in dichloromethane (2 ml), trifluoroacetic acid (2 ml) added and stirred at ambient temperature for 2 hours. The mixture was absorbed onto an SCX-2 column and washed with methanol (2 column volumes) then the product eluted with a 2M solution of ammonia in methanol (2 column volumes) and concentrated under reduced pressure. The residue was suspended in diethyl ether (2 ml), isohexane (2 ml) was added and stirred at ambient temperature for 18 hours. The resulting solid was filtered and dried, to give the title compound (152 mg, >99%); NMR Spectrum: (DMSO-d₆) 1.70 (m, 4H), 2.36 (s, 3H), 2.46-2.57 (m, 6H), 2.75 (t, 2H), 4.91 (br s, 2H), 6.62 (m, 1H), 6.80 (m, 1H), 6.99 (m, 1H), 7.21 (m, 1H), 7.67 (d, 2H), 8.05 (m, 3H), 8.67 (s, 1H), 9.71 (s, 1H), 10.31 (s, 1H);

(CDCl₃) 1.92 (m, 4H), 2.39 (s, 3H), 2.58 (t, 2H), 2.71 (m, 4H), 2.89 (t, 2H), 3.91 (s, 2H), 6.87 (m, 2H), 7.11 (m, 1H), 7.37 (d, 1H), 7.64 (d, 2H), 7.98 (m, 3H), 8.23 (d, 1H), 8.31 (d, 1H), 11.75 (s, 1H); Mass Spectrum: M+H⁺444.

### EXAMPLE 8

### Preparation of (6-(4-{[(2-aminophenyl)amino]carbonyl}phenyl)-5-methyl-N-(2-pyrrolidin-1-ylethyl)nicotinamide)

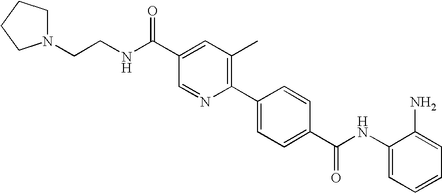

6-{4-[({2-[(tert-Butoxycarbonyl)amino]phenyl}amino)carbonyl]phenyl}-5-methylnicotinic acid (130 mg, 0.29 mmol, see Method 8 below) was suspended in dichloromethane (13 ml) and 1-chloro-N,N, 2-trimethyl-1-propenylamine (78 μl, 0.58 mmol) was added and the resulting solution stirred at ambient temperature for 2 hours. The solvents were evaporated under reduced pressure and the residue dried under high vacuum. The solid obtained was suspended in dichloromethane (10 ml), and then pyridine (70 μl, 0.87 mmol) and 1-(2-aminoethyl)pyrrolidine (66 mg, 0.58 mmol) added. The solution was stirred at ambient temperature for 72 hours then water and saturated aqueous sodium bicarbonate solution added. The dichloromethane layer was washed with brine then absorbed onto an SCX-2 column, washed with a 50% solution of methanol in water (2 column volumes), then methanol (2 column volumes), then the product eluted with a 2M solution of ammonia in methanol (2 column volumes) and concentrated under reduced pressure. The residue was purified by flash column chromatography eluting with a solution of ammonia in methanol (2M) in dichloromethane (10% then 20%). Fractions containing the t-butoxycarbonyl (BOC) protected product were evaporated and re-dissolved in dichloromethane (3 ml), trifluoroacetic acid (3 ml) added and stirred at ambient temperature for 3 hours. The mixture was absorbed onto an SCX-2 column and washed with methanol (2 column volumes) then the product eluted with a 2M solution of ammonia in methanol (2 column volumes) and concentrated under reduced pressure, to give the title compound (7.7 mg, 6%); NMR Spectrum: (DMSO-d₆) 1.71 (m, 4H), 2.42 (s, 3H), 2.56 (m, 4H), 2.65 (m, 2H), 3.44 (m, 2H), 4.93 (br s, 2H), 6.62 (m, 1H), 6.81 (m, 1H), 6.99 (m, 1H), 7.21 (m, 1H), 7.72 (d, 2H), 8.10 (d, 2H), 8.16 (d, 1H), 8.66 (m, 1H), 8.93 (d, 1H), 9.74 (s, 1H); Mass Spectrum: M+H⁺444.

### EXAMPLE 9

### Preparation of (6-(4-{[(2-aminophenyl)amino]carbonyl}phenyl)-N-[2-(dimethylamino)ethyl]-5-methylnicotinamide (9.1) (6-(4-{[(2-aminophenyl)amino]carbonyl}phenyl)-N-[2-(dimethylamino)ethyl]-5-N-methyl-methylnicotinamide (9.2) and (6-(4-{[(2-aminophenyl)amino]carbonyl}phenyl-N-[2-(diethylamino)ethyl]-5-methylnicotinamide) (9.3)

6-{4-[({2-[(tert-butoxycarbonyl)amino]phenyl}amino)carbonyl]phenyl}-5-methylnicotinic acid (698 mg, 1.56 mmol, see Method 8 below) was suspended in dichloromethane (30 ml) and 1-chloro-N,N,2-trimethyl-1-propenylamine (413 μl, 3.12 mmol) was added and the resulting solution stirred at ambient temperature for 2 hours. The solvents were evaporated under reduced pressure and the residue dried under high vacuum for 20 minutes. The residue was dissolved in dichloromethane (40 ml), and 10 mls transferred into separate reaction vessels. To each of the vessels was added pyridine (95 μl, 1.17 mmol) followed by the appropriate amine (0.78 mmol, see table below), and the solutions stirred at ambient temperature for 18 hours. Each reaction was treated in an identical manner as outlined below.

The mixture was absorbed onto an SCX-2 column, washed with methanol (2 column volumes), then the product eluted with a 2M solution of ammonia in methanol (2 column volumes) and concentrated under reduced pressure. The residue was re-dissolved in dichloromethane (5 ml), trifluoroacetic acid (5 ml) added and stirred at ambient temperature for 3 hours. The mixture was absorbed onto an SCX-2 column and washed with methanol (2 column volumes) then the product eluted with a 2M solution of ammonia in methanol (2 column volumes) and concentrated under reduced pressure. The residue was purified by acid modified reverse phase HPLC. Fractions containing the product were absorbed onto an SCX-2 column and washed with methanol (2 column volumes) then the products eluted with a 2M solution of ammonia in methanol (2 column volumes) and concentrated under reduced pressure, to give the title compounds.

TABLE

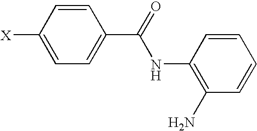

| Compd | X | Analytical Data | SM |
|---|---|---|---|
| 9.1 | 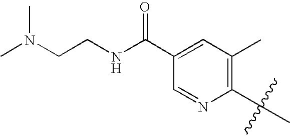 | NMR Spectrum: (DMSO-d₆) 2.21 (s,6H), 2.42 (s, 3H), 2.45 (m, 2H), 3.41(m, 2H), 4.92 (br s, 2H), 6.62 (m,1H), 6.81 (m, 1H), 6.99 (m, 1H),7.21 (m, 1H), 7.72 (d, 2H), 8.10 (d,2H), 8.16 (s, 1H), 8.59 (m, 1H), 8.93(s, 1H), 9.74 (s, 1H); Mass Spectrum:M + H⁺ 418. | Method 8 |
| 9.2 | 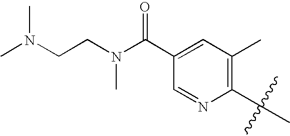 | NMR Spectrum: (CDCl₃) 2.14 (s,3H), 2.33 (s, 3H), 2.40 (s, 3H), 2.48(s, 1H), 2.62 (s, 1H), 3.11 (s, 3H),3.42 (s, 1H), 3.69 (s, 1H), 3.91 (s,2H), 6.88 (m, 2H), 7.12 (m, 1H),7.39 (d, 1H), 7.66 (d, 2H), 7.71 (s,1H), 8.02 (m, 3H), 8.61 (s, 1H);Mass Spectrum: M + H⁺ 432. | Method 8 |
| 9.3 | 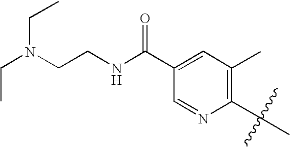 | NMR Spectrum: (DMSO-d₆) 0.99 (t,6H), 2.42 (s, 3H), 2.57 (m, 6H), 3.36(m, 2H), 4.92 (br s, 2H), 6.62 (m,1H), 6.81 (m, 1H), 6.99 (m, 1H),7.21 (m, 1H), 7.72 (d, 2H), 8.10 (d,2H), 8.15 (s, 1H), 8.59 (m, 1H), 8.92(s, 1H), 9.74 (s, 1H); Mass Spectrum:M + H⁺ 446. | Method 8 |

### EXAMPLE 10

### Preparation of (N-(2-aminophenyl)-4-(3-ethylpyridin-2-yl)benzamide)

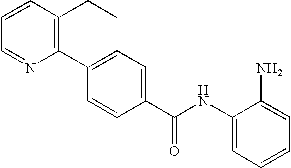

tert-Butyl (2-{[4-(3-ethylpyridin-2-yl)benzoyl]amino}phenyl) carbamate (Method 10, 137 mg, 0.33 mmol) was taken up in dichloromethane (4 ml) and trifluoroacetic acid (1 ml) added. The reaction mixture was allowed to stir overnight. The reaction mixture was then diluted with methanol before pouring directly onto a 5 g isolute SCX-2 cartridge. The cartridge was washed with methanol. Products were then eluted from the cartridge with a 2M solution of ammonia in methanol. Evaporation to dryness and trituration of the residue with diethyl ether and isohexane afforded the title compound (72 mg, 69%); NMR Spectrum: (DMSO-d₆) □1.10 (m, 3H), 2.67 (q, 2H), 4.93 (s, 2H), 6.62 (m, 1H), 6.81 (m, 1H), 6.99 (m, 1H), 7.21 (m, 1H), 7.39 (m, 1H), 7.61 (d, 2H), 7.81 (m, 1H), 8.08 (d, 2H), 8.52 (m, 1H), 9.73 (s, 1H); Mass Spectrum: M+H⁺318.

### EXAMPLE 11

### Preparation of (N-(2-aminophenyl)-4-{3-methyl-5-[(4-(2-methoxyethyl)-piperazin-1-yl)methyl]pyridin-2-yl}benzamide)

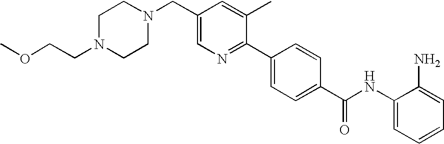

tert-Butyl (2-{[4-(5-formylpyridin-2-yl)benzoyl]amino}phenyl)carbamate (0.20 g, 0.4634 mmol; prepared as described in method 3), 1-(2-methoxyethyl)-piperazine (104 mg, 0.7 mmol) and acetic acid (27 μl, 0.4634 mmol) were dissolved in tetrahydrofuran (5 ml). The mixture was stirred at ambient temperature for 1 hour then sodium triacetoxyborohydride (147 mg, 0.695 mmol) added and the mixture stirred for a further 16 hours. The mixture was concentrated and partitioned between dichloromethane (5 ml) and saturated sodium bicarbonate (5 ml). The organics were washed with further saturated sodium bicarbonate (2×5 ml) and brine (5 ml) then dried over magnesium sulphate and filtered. This solution was then treated with trifluoroacetic acid (1.0 ml) and the solution then stirred at ambient temperature for 2 hours. The resulting solution was absorbed onto an SCX-2 column, which was washed with methanol (3 column volumes) and the product eluted with a 2M solution of ammonia in methanol (3 column volumes) which was concentrated to give the product as a gum. The residue was purified by acid modified reverse phase HPLC. Fractions containing the product were absorbed onto an SCX-2 column and washed with methanol (2 column volumes) then the products eluted with a 2M solution of ammonia in methanol (2 column volumes) and concentrated under reduced pressure, to give a residue which was triturated with ether to give title compound as a solid (12 mg, 6%). NMR Spectrum: (DMSO-d₆); 2.37 (s, 3H), 2.43 (m, 10H), 3.24 (s, 3H), 3.43 (t, 2H), 3.53 (s, 2H), 4.92 (s, 2H), 6.62 (m, 1H), 6.80 (m, 1H), 6.99 (m, 1H), 7.21 (m, 1H), 7.66 (s, 1H), 7.69 (d, 2H), 8.07 (d, 2H), 8.42 (m, 1H), 9.72 (s, 1H). Mass Spectrum: M+H⁺460.

### EXAMPLE 12

Using a method similar to that used in Example 11 the appropriate amine was reacted with tert-butyl (2-{[4-(5-formylpyridin-2-yl)benzoyl]amino}phenyl)carbamate to give the required products.

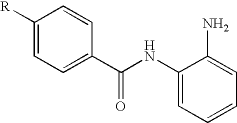

| | R | Spectral Data |
|---|---|---|
| 12.1 | 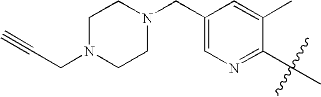 | NMR Spectrum: (DMSO-d₆); 2.38(s, 3H), 2.50 (m, 10H), 3.16 (m, 1H),3.53 (s, 2H), 4.92 (s, 2H), 6.62 (m,1H), 6.80 (m, 1H), 6.99 (m, 1H), 7.21(m, 1H), 7.69 (m, 3H), 8.08 (d, 2H),8.43 (m, 1H), 9.72 (s, 1H). MassSpectrum: M + H⁺ 440. |
| 12.2 | 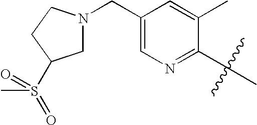 | NMR Spectrum: (DMSO-d₆); 2.14(m, 2H), 2.37 (s, 3H), 2.65 (m, 2H),2.87 (m, 2H), 2.95 (s, 3H), 3.69 (s,2H), 3.82 (m, 1H), 4.92 (s, 2H), 6.62(m, 1H), 6.80 (m, 1H), 6.99 (m, 1H),7.21 (m, 1H), 7.69 (m, 3H), 8.08 (d,2H), 8.46 (m, 1H), 9.72 (s, 1H).Mass Spectrum: M + H⁺ 465. |

### EXAMPLE 13

### Preparation of (N-(2-aminophenyl)-4-(5-{[(2-methoxyethyl-amino]methyl}-3-methylpyridin-2-yl)benzamide)

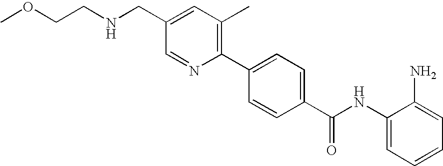

tert-Butyl (2-{[4-(5-formylpyridin-2-yl)benzoyl]amino}phenyl)carbamate (0.20 g, 0.4634 mmol; prepared as described in Method 3) and (2-methoxyethyl)amine (0.7 mmol) was dissolved in dichloromethane (5 ml). Titanium (IV) isopropoxide (0.28 ml, 0.9 mmol) was added and the mixture stirred at ambient temperature for 2 hours. Sodium borohydride (70 mg, 1.85 mmol) and methanol (0.5 ml) was added and the mixture stirred for a further hour. Water (4 ml) and a saturated aqueous sodium bicarbonate solution (2 ml) was added and stirred for 10 minutes. The product was extracted with dichloromethane (3×10 ml) and the organic extracts concentrated to give a residue. The residue was purified using chromatography eluting with 10% methanol in ethyl acetate then concentration of the relevant fractions gave product as a gum. The gum was dissolved in dichloromethane (5 ml), trifluoroacetic acid (1 ml) added and the solution then stirred at ambient temperature for 2 hours. The resultant solution was absorbed onto an SCX-2 column washed with methanol (3 column volumes) and the product eluted with a 2M solution of ammonia in methanol (3 column volumes) which after concentration gave the product as a gum. This was triturated with diethyl ether (3 ml) to give the product as a solid (93 mg, 51%). NMR Spectrum: (DMSO-d₆); 2.37 (s, 3H), 2.75 (m, 2H), 3.27 (s, 3H), 3.31 (s, 3H), 3.46 (m, 2H), 3.83 (s, 2H), 4.92 (s, 2H), 6.62 (m, 1H), 6.81 (m, 1H), 6.99 (m, 1H), 7.21 (m, 1H), 7.68 (d, 2H), 7.72 (s, 1H), 8.08 (d, 2H), 8.48 (s, 1H), 9.73 (s, 1H). Mass Spectrum: M+H⁺391

### EXAMPLE 14

Using a method similar to that used in Example 13 the appropriate amine was reacted with tert-butyl (2-{[4-(5-formylpyridin-2-yl)benzoyl]amino}phenyl)carbamate to give the required products.

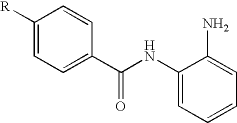

| No. | R | Spectral Data |
|---|---|---|
| 14.1 | 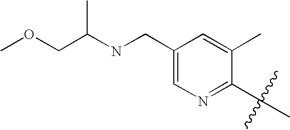 | NMR Spectrum: (DMSO-d₆); 1.09(m, 3H), 2.37 (s, 3H), 2.94 (m, 1H),3.39 (m, 5H), 3.89 (m, 2H), 4.92 (s,2H), 6.62 (m, 1H), 6.80 (m, 1H),6.98 (m, 1H), 7.21 (m, 1H), 7.68 (d,2H), 7.74 (s, 1H), 8.08 (d, 2H), 8.50(s, 1H), 9.72 (s, 1H). MassSpectrum: M + H⁺ 405. |
| 14.2 | 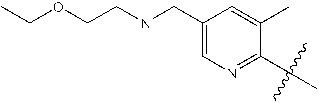 | NMR Spectrum: (DMSO-d₆); 1.12(t, 3H), 2.37 (s, 3H), 2.71 (m, 2H),2.88 (br s, 1H), 3.45 (m, 4H), 3.80(s, 2H), 4.92 (s, 2H), 6.62 (m, 1H),6.81 (m, 1H), 6.99 (m, 1H), 7.21 (m,1H), 7.69 (m, 3H), 8.07 (d, 2H),8.46 (s, 1H), 9.72 (s, 1H). MassSpectrum: M + H⁺ 405. |
| 14.3 | 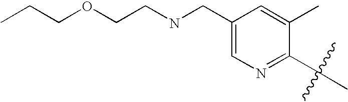 | NMR Spectrum: (DMSO-d₆); 0.88(t, 3H), 1.53 (m, 2H), 2.37 (s, 3H),2.76 (m, 2H), 3.36 (m, 2H), 3.50 (m,2H), 3.85 (s, 2H), 4.92 (s, 2H), 6.62(m, 1H), 6.81 (m, 1H), 6.99 (m, 1H),7.21 (m, 1H), 7.68 (d, 2H), 7.73 (s,1H), 8.08 (d, 2H), 8.48 (m, 1H),9.72 (s, 1H). Mass Spectrum:M + H⁺ 418. |
| 14.4 | 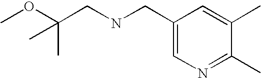 | NMR Spectrum: (DMSO-d₆); 1.14(s, 6H), 2.37 (s, 3H), 2.55 (s, 2H),3.10 (s, 3H), 3.84 (s, 2H), 4.92 (s,2H), 6.62 (m, 1H), 6.81 (m, 1H),6.99 (m, 1H), 7.21 (m, 1H), 7.69 (d,2H), 7.73 (s, 1H), 8.08 (d, 2H), 8.49(s, 1H), 9.72 (s, 1H). MassSpectrum: M + H⁺ 419. |
| 14.5 | 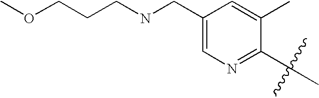 | NMR Spectrum: (DMSO-d₆); 1.69(m, 2H), 2.37 (s, 3H), 2.59 (m, 2H),3.23 (s, 3H), 3.39 (m, 2H), 3.76 (s,2H), 4.92 (s, 2H), 6.62 (m, 1H), 6.807.68 (m, 3H), 8.07 (d, 2H), 8.46 (m,1H), 9.72 (s, 1H). Mass Spectrum:M + H⁺ 405. |
| 14.6 | 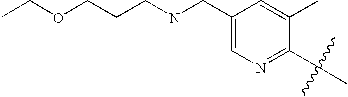 | NMR Spectrum: (DMSO-d₆); 1.10(t, 3H), 1.68 (m, 2H), 2.36 (s, 3H),2.59 (m, 2H), 3.41 (m, 4H), 3.75 (s,2H), 4.92 (s, 2H), 6.62 (m, 1H), 6.80(m, 1H), 6.99 (m, 1H), 7.21 (m, 1H),7.68 (m, 3H), 8.07 (d, 2H), 8.46 (m,1H), 9.72 (s, 1H). Mass Spectrum:M + H⁺ 419. |
| 14.7 | 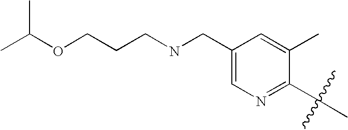 | NMR Spectrum: (DMSO-d₆); 1.07(d, 6H), 1.65 (m, 2H), 2.36 (s, 3H),2.59 (m, 2H), 3.42 (m, 2H), 3.50 (m,1H), 3.75 (s, 2H), 4.92 (s, 2H), 6.62(m, 1H), 6.81 (m, 1H), 6.99 (m, 1H),7.21 (m, 1H), 7.68 (m, 3H), 8.07 (d,2H), 8.46 (m, 1H), 9.72 (s, 1H).Mass Spectrum: M + H⁺ 432. |

### EXAMPLE 15

### Preparation of (N-(2-aminophenyl)-4-{3-methyl-5-[(methylamino)methyl]pyridin-2-yl}benzamide)

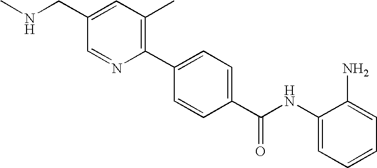

tert-Butyl[2-({4-[5-(hydroxymethyl)pyridin-2-yl]benzoyl}amino)phenyl]carbamate (0.2 g, 0.461 mmol, prepared a described in Method 2) was dissolved with stirring in tetrahydrofuran (6 ml). N,N-diisopropylethylamine (0.24 ml, 1.38 mmol) was added and the mixture cooled to 0° C. in an ice bath. Methanesulfonyl chloride (47.5 μl, 0.61 mmol) was added and the solution stirred for 30 minutes at low temperature. The mixture was allowed to warm to ambient temperature and a solution of methylamine (7.0 mmol) in tetrahydrofuran (1 ml) was added. The mixture was then stirred at ambient temperature for 16 hours. The solution was concentrated under reduced pressure and the residue purified by chromatography eluting with 5% methanol in dichloromethane which after concentration gave the product as a gum. The residue was dissolved in dichloromethane (5 ml), trifluoroacetic acid (1 ml) added and the solution stirred at ambient temperature for 2 hours. The resulting solution was absorbed onto an SCX-2 column, which was washed with methanol (3 column volumes) and the product eluted with a 2M solution of ammonia in methanol (3 column volumes) which after concentration gave the product as a gum. This was triturated with diethyl ether (3 ml) to give the title compound as a solid (187 mg, 36%).

### NMR Spectrum: (DMSO-d₆); 2.33 (s, 3H), 2.37 (s, 3H), 3.72 (s, 2H), 4.92 (s, 2H), 6.62 (m, 1H), 6.80 (m, 1H), 6.99 (m, 1H), 7.21 (m, 1H), 7.68 (m, 3H), 8.07 (d, 2H), 8.46 (s, 1H), 9.72 (s, 1H). Mass Spectrum: M+H⁺347.

### EXAMPLE 16

Using a method similar to that used in Example 15 the appropriate amine was reacted with tert-butyl[2-({4-[5-(hydroxymethyl)pyridin-2-yl]benzoyl}amino)phenyl]carbamate to give the required products.

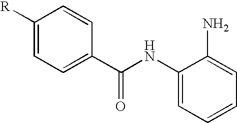

| | R | Spectral Data |
|---|---|---|
| 16.1 | 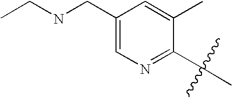 | NMR Spectrum: (DMSO-d₆); 1.07 (t,3H), 2.37 (s, 3H), 2.60 (m, 2H), 3.77 (s,2H), 4.92 (s, 2H), 6.62 (m, 1H), 6.81 (m,1H), 6.99 (m, 1H), 7.21 (m, 1H), 7.69 (m,3H), 8.07 (d, 2H), 8.46 (m, 1H), 9.72 (s,1H). Mass Spectrum: M + H⁺ 361. |
| 16.2 | 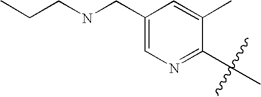 | NMR Spectrum: (CDCl₃); 0.95 (t, 3H),1.57 (m, 2H), 2.36 (s, 3H), 2.65 (m, 2H),3.84 (s, 2H), 3.88 (s, 2H), 6.86 (m, 2H),7.10 (m, 1H), 7.37 (m, 1H), 7.63 (m, 3H),7.97 (m, 3H), 8.47 (s, 1H). MassSpectrum: M + H⁺ 375. |
| 16.3 | 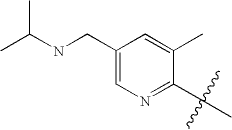 | NMR Spectrum: (DMSO-d₆); 1.05 (d,6H), 2.36 (s, 3H), 2.77 (m, 1H), 3.77 (s,2H), 4.92 (s, 2H), 6.62 (m, 1H), 6.80 (m,1H), 6.99 (m, 1H), 7.21 (m, 1H), 7.69 (m,3H), 8.07 (d, 2H), 8.47 (m, 1H), 9.72 (s,1H). Mass Spectrum: M + H⁺ 375. |
| 16.4 | 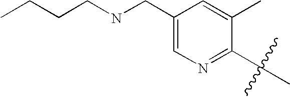 | NMR Spectrum: (DMSO-d₆); 0.89 (t,3H), 1.34 (m, 2H), 1.45 (m, 2H), 2.36 (s,3H), 2.55 (m, 2H), 3.76 (s, 2H), 4.92 (s,2H), 6.62 (m, 1H), 6.80 (m, 1H), 6.99 (m,1H), 7.21 (m, 1H), 7.68 (m, 3H), 8.07 (d,2H), 8.46 (m, 1H), 9.72 (s, 1H). MassSpectrum: M + H⁺ 389. |
| 16.5 | 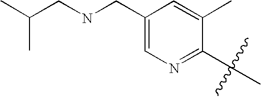 | NMR Spectrum: (CDCl₃) □0.97 (d, 6H),1.84 (m, 1H), 2.39 (s, 3H), 2.53 (m, 2H),3.88 (m, 4H), 6.89 (m, 2H), 7.12 (m, 1H),7.40 (m, 1H), 7.66 (m, 3H), 7.88 (s, 1H),8.00 (d, 2H), 8.51 (m, 1H). MassSpectrum: M + H⁺ 389 |
| 16.6 | 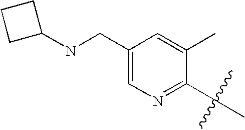 | NMR Spectrum: (DMSO-d₆); 1.54—1.79 (m, 4H), 2.11 (m, 2H), 2.36 (s, 3H),3.22 (m, 1H), 3.68 (s, 2H), 4.92 (s, 2H),6.62 (m, 1H), 6.80 (m, 1H), 6.99 (m, 1H),7.21 (m, 1H), 7.68 (m, 3H), 8.07 (d, 2H),8.45 (m, 1H), 9.72 (s, 1H). MassSpectrum: M + H⁺ 387. |
| 16.7 | 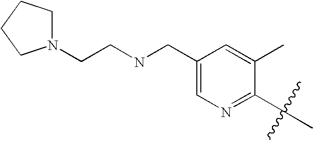 | NMR Spectrum: (DMSO-d₆); 1.74 (m,4H), 2.37 (s, 3H), 2.66 (m, 8H), 3.81 (s,2H), 4.92 (s, 2H), 6.62 (m, 1H), 6.80 (m,1H), 6.99 (m, 1H), 7.21 (m, 1H), 7.68 (d,2H), 7.72 (m, 1H), 8.07 (d, 2H), 8.48 (m,1H), 9.72 (s, 1H). Mass Spectrum:M + H⁺ 430. |
| 16.8 | 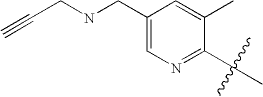 | NMR Spectrum: (DMSO-d₆); 2.37 (s,3H), 3.25 (s, 1H), 3.47 (m, 2H), 3.90 (s,2H), 4.91 (s, 2H), 6.62 (m, 1H), 6.81 (m,1H), 6.99 (m, 1H), 7.21 (m, 1H), 7.70 (m,3H), 8.08 (d, 2H), 8.48 (s, 1H), 9.73 (s,1H). Mass Spectrum: M + H⁺ 371. |
| 16.9 | 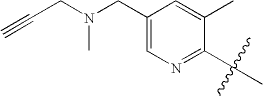 | NMR Spectrum: (DMSO-d₆); 2.27 (s,3H), 2.37 (s, 3H), 3.24 (s, 1H), 3.37 (s,2H), 3.61 (s, 2H), 4.95 (s, 2H), 6.62 (m,1H), 6.80 (m, 1H), 6.99 (m, 1H), 7.21 (m,1H), 7.69 (m, 3H), 8.08 (d, 2H), 8.43 (s,1H), 9.72 (s, 1H). Mass Spectrum:M + H⁺ 385. |
| 16.10 | 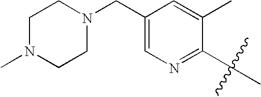 | NMR Spectrum: (DMSO-d₆); 2.24 (m,3H), 2.37 (s, 3H), 2.45 (m, 8H), 3.54 (s,2H), 4.92 (s, 2H), 6.62 (m, 1H), 6.80 (m,1H), 6.99 (m, 1H), 7.21 (m, 1H), 7.68 (m,3H), 8.07 (d, 2H), 8.43 (m, 1H), 9.72 (s,1H). Mass Spectrum: M + H⁺ 416. |
| 16.11 | 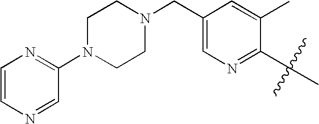 | NMR Spectrum: (DMSO-d₆); 2.39 (s,3H), 2.59 (m, 4H), 3.56 (m, 6H), 4.94 (s,2H), 6.62 (m, 1H), 6.81 (m, 1H), 6.99 (m,1H), 7.21 (m, 1H), 7.72 (m, 3H), 7.86 (m,1H), 8.09 (m, 3H), 8.33 (s, 1H), 8.50 (s,1H), 9.73 (s, 1H). Mass Spectrum:M + H⁺ 480. |

### EXAMPLE 17

### Preparation of (N-(2-aminophenyl)-4-[3-methyl-5-(pyrrolidin-1-ylmethyl)pyridin-2-yl]benzamide)

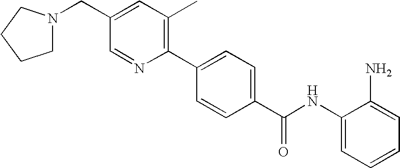

tert-Butyl (2-{[4-(5-formylpyridin-2-yl)benzoyl]amino}phenyl)carbamate (200 mg, 0.46 mmol; prepared as described in method 3), pyrrolidine (49.4 mg, 0.70 mmol) and acetic acid (0.027 ml, 0.46 mmol) were stirred at ambient temperature in tetrahydrofuran (5 ml) for 1 hour. Sodium triacetoxyborohydride (147 mg, 0.70 mmol) was added and the mixture stirred at ambient temperature for 3 hour. The solvent was evaporated and the residue partitioned between dichloromethane and saturated sodium bicarbonate solution. The organic solution was washed once with brine, dried over magnesium sulphate, filtered and the solvent evaporated. The residual gum was purified by silica chromatography eluting with ammonia in MeOH (2M) DCM (5%). Concentration of the solvent gave the product as a gum. This was dissolved in dichloromethane (4 ml) and trifluoroacetic acid (1 ml) was added and the solution then stirred at ambient temperature for 3 hours. The resulting solution was absorbed onto an SCX-2 column which was washed with methanol (2 column volumes) and then eluted with a 2M solution of ammonia in methanol (2 column volumes) the concentrated to give the product as a gum. This was stirred in diethyl ether to give the title compound as a white solid (85 mg, 100%). NMR Spectrum: (DMSO-d₆); 1.74 (m, 4H), 2.37 (s, 3H), 2.51 (m, 4H), 3.66 (s, 2H), 4.92 (brs, 2H), 6.62 (m, 1H), 6.81 (d, 1H), 6.99 (m, 1H), 7.21 (d, 1H), 7.69 (m, 3H), 8.07 (d, 2H), 8.44 (s, 1H), 9.72 (s, 1H); Mass Spectrum: M+H⁺387.

### EXAMPLE 18

By a method analogous to that used in Example 17 reacting the appropriate amine with tert-butyl (2-{[4-(5-formylpyridin-2-yl)benzoyl]amino}phenyl)carbamate to give the required products.

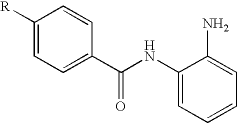

| Compound | R | Analytical Data | SM |
|---|---|---|---|
| 18.1 | 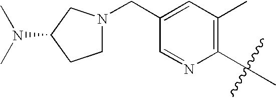 | NMR Spectrum: (DMSO-d₆); 1.64(m, 1H), 1.88 (m, 1H), 2.11 (s, 6H),2.37 (s, 3H), 2.40 (m, 2H), 2.62 (m,1H), 2.74 (m, 2H), 3.57 (d, 1H), 3.66(d, 1H), 4.92 (bs, 2H), 6.62 (m, 1H),6.80 (d, 1H), 6.99 (m, 1H), 7.21 (d,1H), 7.66 (s, 1H), 7.69 (d, 2H), 8.08(d, 2H), 8.43 (s, 1H), 9.72 (s, 1H);Mass Spectrum: M + H⁺ 430. | |
| 18.2 | 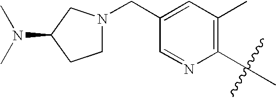 | NMR Spectrum: (DMSO-d₆); 1.64(m, 1H), 1.88 (m, 1H), 2.11 (s, 6H),2.37 (s, 3H), 2.40 (m, 2H), 2.62 (m,1H), 2.74 (m, 2H), 3.57 (d, 1H), 3.66(d, 1H), 4.92 (bs, 2H), 6.62 (m, 1H),6.80 (d, 1H), 6.99 (m, 1H), 7.21 (d,1H), 7.66 (s, 1H), 7.69 (d, 2H), 8.07(d, 2H), 8.43 (s, 1H), 9.72 (s, 1H);Mass Spectrum: M + H⁺ 430. | |

### METHODS

### Method 1

### Preparation of t-butyl (2-{[4-(5-{[(cyclopropylmethyl)amino]methyl}-3-methylpyridin-2-yl)-benzoyl]amino}phenyl)carbamate

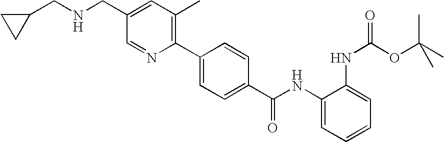

t-Butyl [2-({4-[5-(hydroxymethyl)-3-methylpyridin-2-yl]benzoyl}amino)phenyl]-carbamate (1.46 g, 3.37 mmol, see Method 2 below) was stirred and dissolved in chloroform (75 ml). Pyridine (3 drops) was added followed by thionylchloride (0.42 ml, 5.73 mmol) and the solution then stirred for 1 hour at ambient temperature. 2-Methyl-2-propanol (1.42 g, 16.85 mmol) was then added and the solution stirred for 2 hours at ambient temperature. The solution was then concentrated under reduced pressure and the solid dissolved in dimethylformamide (20 ml). Aminomethylcyclopropane (8.8 ml, 101 mmol) was added and the solution heated at 50° C. for 18 hours. The solution was concentrated under reduced pressure and added to water (100 ml) then stirred at ambient temperature. The solid was filtered and washed with water and dried in vacuo at 50° C. for 24 hours to give the title compound (878 mg, 54%); NMR Spectrum: (DMSO-d₆) 0.40 (m, 2H), 0.58 (m, 2H), 1.15 (m, 1H), 1.44 (s, 9H), 2.38 (s, 3H), 2.85 (d, 2H), 4.19 (s, 2H), 7.18 (m, 2H), 7.56 (m, 2H), 7.71 (d, 2H), 8.01 (s, 1H), 8.08 (d, 2H), 8.67 (s, 1H), 8.72 (s, 1H), 9.99 (s, 1H); Mass Spectrum: M+H⁺487.

### Method 2

### Preparation of t-butyl [2-({4-[5-(hydroxymethyl-3-methylpyridin-2-yl]benzoyl}amino)phenyl]carbamate

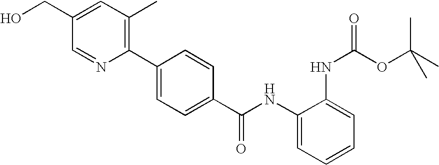

t-Butyl (2-{[4-(5-formyl-3-methylpyridin-2-yl)benzoyl]amino}phenyl)carbamate (2 g, 4.64 mmol, see Method 3 below) was stirred and suspended in methanol (100 ml) then sodium borohydride (350 mg, 9.28 mmol) added in portions and the solution stirred for 2 hours at ambient temperature. The solution was concentrated under reduced pressure and the residue partitioned between saturated sodium bicarbonate solution (100 ml) and dichloromethane. This was extracted with dichloromethane, the combined organic extracts were washed with brine and dried over magnesium sulphate. The solid was filtered and the solution concentrated under reduced pressure to give the title compound (2.04 g, 100%); NMR Spectrum: (DMSO-d₆) 1.46 (s, 9H), 2.37 (s, 3H), 4.58 (d, 2H), 5.32 (t, 1H), 7.20 (m, 2H), 7.58 (m, 2H), 7.68 (s, 1H), 7.72 (d, 2H), 8.05 (d, 2H), 8.47 (s, 1H), 8.69 (s, 1H), 9.90 (s, 1H); Mass Spectrum: M+H⁺434.

### Method 3

### Preparation of t-butyl (2-{[4-(5-formyl-3-methylpyridin-2-yl)benzoyl]amino}phenyl carbamate

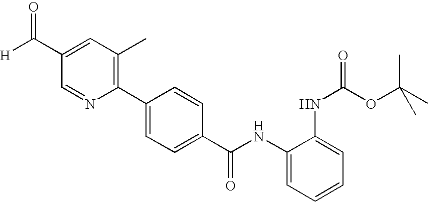

N-(2-t-Butoxycarbonylaminophenyl)-4-(4,4,5,5-tetramethyl-1,3,2,-dioxaborolan-2-yl)benzamide (7.69 g, 17.55 mmol—prepared as described in Method 13, page 60, of International patent publication number WO 03/087057), was added to a stirred solution of 6-bromo-5-methylnicotinaldehyde (3.51 g, 17.55 mmol, see Method 4 below) in dimethoxyethane (100 ml) at ambient temperature under a nitrogen atmosphere. 1,1′Bis(diphenylphosphino)ferrocenedichloropalladium(II) (0.72 g, 0.88 mmol) was added followed by saturated aqueous sodium bicarbonate solution (50 ml) and the mixture heated at 60° C. for 2 hours. The reaction mixture was concentrated under reduced pressure and the residue partitioned between dichloromethane and water. The dichloromethane layer was washed with saturated aqueous sodium bicarbonate solution and brine, then dried over magnesium sulphate, filtered and concentrated under reduced pressure. The residue was purified by flash chromatography, eluting with 40% ethyl acetate in isohexane, to give the title compound (5.93 g, 75%); NMR Spectrum: (DMSO-d₆) 1.46 (s, 9H), 2.47 (s, 3H), 7.20 (m, 2H), 7.58 (m, 2H), 7.80 (d, 2H), 8.09 (d, 2H), 8.23 (s, 1H), 8.70 (s, 1H), 9.04 (s, 1H), 9.94 (s, 1H), 10.17 (s, 1H); Mass Spectrum: M+H⁺432.

### Method 4

### Preparation of 6-bromo-5-methylnicotinaldehyde

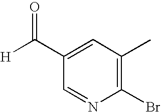

2,5-Dibromo-3-picoline (5.1 g, 20.30 mmol) in tetrahydrofuran (25 ml) was added dropwise to a 2M solution of isopropylmagnesium chloride (10.7 ml, 21.3 mmol) in tetrahydrofuran at 0° C. The solution was stirred for 2 hours at 0° C. and then for 1 hour at ambient temperature. A solution of 4-formylmorpholine (2.1 ml, 20.3 mmol) in tetrahydrofuran (25 ml) was added dropwise and the solution stirred at ambient temperature for 1 hour. The solution was poured into water and extracted with ethyl acetate. The combined organic extracts were washed with brine, dried over magnesium sulphate, filtered and the solution concentrated under reduced pressure. The residue was purified by flash chromatography, eluting with 10% ethyl acetate in isohexane, to give the title compound (3.0 g, 74%); NMR Spectrum: (DMSO-d₆) 2.44 (s, 3H), 8.19 (s, 1H), 8.73 (s, 1H), 10.09 (s, 1H).

### Method 5

### Preparation of tert-butyl {2-[(4-{5-[(3-bromopropanoyl)amino]-3-methylpyridin-2-yl}benzoyl)amino]phenyl}carbamate

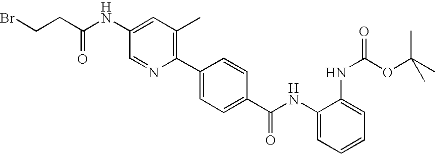

tert-Butyl (2-{[4-(5-amino-3-methylpyridin-2-yl)benzoyl]amino}phenyl) carbamate (250 mg, 0.60 mmol, see Method 6 below) was dissolved in dichloromethane (5 ml) and then pyridine (145 μl, 1.79 mmol) and 3-bromopropionyl chloride (90 μl, 0.90 mmol) added. The solution was stirred at ambient temperature for 18 hours then saturated aqueous sodium bicarbonate solution (5 ml) added. The dichloromethane layer was filtered through a PTFE cup and evaporated under reduced pressure. The residue was purified by flash chromatography, eluting with 40% ethyl acetate in isohexane, then 5% methanol in dichloromethane, to give the title compound (189 mg, 57%); Mass Spectrum: M+H⁺555. Used directly in next step.

### Method 6

### Preparation of tert-butyl (2-{[4-(5-amino-3-methylpyridin-2-yl)benzoyl]amino}phenyl)carbamate

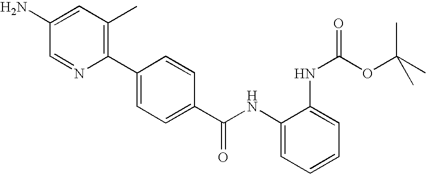

tert-butyl (2-{[4-(3-methyl-5-nitropyridin-2-yl)benzoyl]amino}phenyl)carbamate (1.81 g, 4.03 mmol, see Method 7 below) was dissolved in methanol (100 ml) and a catalytic amount of palladium hydroxide (20% on carbon) powder, added. The mixture was stirred at ambient temperature under an atmosphere of hydrogen for 4 hours then filtered and evaporated under reduced pressure. The residue was purified by flash chromatography, eluting with methanol in dichloromethane (2% then 5%), to give the title compound (1.35 g, 80%); NMR Spectrum: (DMSO-d₆) 1.46 (s, 9H), 2.27 (s, 3H), 5.40 (s, 2H), 6.86 (d, 1H), 7.19 (m, 2H), 7.56 (m, 2H), 7.64 (d, 2H), 7.91 (d, 1H), 7.98 (d, 2H), 8.68 (s, 1H), 9.85 (s, 1H); Mass Spectrum: M+H⁺419.

### Method 7

### Preparation of tert-butyl (2-{[4-(3-methyl-5-nitropyridin-2-yl)benzoyl]amino}phenyl)carbamate

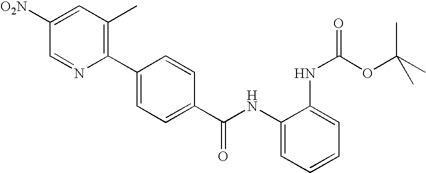

N-(2-t-Butoxycarbonylaminophenyl)-4-(4,4,5,5-tetramethyl-1,3,2,-dioxaborolan-2-yl)benzamide (2.45 g, 5.59 mmol—prepared as described in Method 13, page 60, of International patent publication number WO 03/087057), was added to a stirred solution of 2-chloro-3-methyl-5-nitropyridine (964 mg, 5.59 mmol) in dimethoxyethane (50 ml) at ambient temperature under a nitrogen atmosphere. 1,1′Bis(diphenylphosphino)ferrocenedichloropalladium(II) (0.23 g, 0.28 mmol) was added followed by saturated aqueous sodium bicarbonate solution (25 ml) and the mixture heated at 80° C. for 1 hour. The reaction mixture was concentrated under reduced pressure and the residue partitioned between dichloromethane (200 ml) and water (100 ml). The dichloromethane layer was washed with brine then dried over magnesium sulphate, filtered and concentrated under reduced pressure. The residue was purified by flash chromatography, eluting with 30% ethyl acetate in isohexane, to give the title compound (1.81 g, 72%); NMR Spectrum: (DMSO-d₆) 1.46 (s, 9H), 2.50 (s, 3H), 7.20 (m, 2H), 7.57 (m, 2H), 7.82 (d, 2H), 8.11 (d, 2H), 8.66 (d, 1H), 8.74 (s, 1H), 9.33 (d, 1H), 9.97 (s, 1H); Mass Spectrum: M+H⁺449.

### Method 8

### Preparation of 6-{4-[([2-[(tert-butoxycarbonyl)amino]phenyl]amino)carbonyl]phenyl}-5-methylnicotinic acid

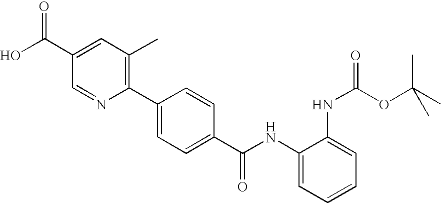

N-(2-t-Butoxycarbonylaminophenyl)-4-(4,4,5,5-tetramethyl-1,3,2,-dioxaborolan-2-yl)benzamide (1.55 g, 3.54 mmol—prepared as described in Method 13, page 60, of International patent publication number WO 03/087057), was added to a stirred solution of 6-bromo-5-methylnicotinic acid (764 mg, 3.54 mmol, see Method 9 below) in dimethoxyethane (50 ml) at ambient temperature under a nitrogen atmosphere.

1,1′Bis(diphenylphosphino)ferrocenedichloropalladium(II) (144 mg, 0.18 mmol) was added followed by saturated aqueous sodium bicarbonate solution (50 ml) and the mixture heated at 80° C. for 2 hours. The reaction mixture was concentrated under reduced pressure and the residue partitioned between ethyl acetate (100 ml) and water (100 ml). The aqueous layer was acidified to pH 3 with a 2M aqueous solution of hydrochloric acid and extracted with ethyl acetate (100 ml×2). The combined organics were washed with brine then dried over magnesium sulphate, filtered and concentrated under reduced pressure. The residue was triturated with diethyl ether and the resulting solid filtered, to give the title compound (828 mg, 52%); NMR Spectrum: (DMSO-d₆) 1.46 (s, 9H), 2.44 (s, 3H), 7.20 (m, 2H), 7.57 (m, 2H), 7.78 (d, 2H), 8.08 (d, 2H), 8.25 (d, 1H), 8.69 (s, 1H), 9.01 (d, 1H), 9.92 (s, 1H), 13.37 (br s, 1H); Mass Spectrum: M+H⁺448.

### Method 9

### Preparation of 6-bromo-5-methylnicotinic acid

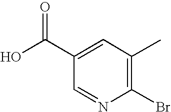

2,5-Dibromo-3-picoline (5.07 g, 20.21 mmol) in tetrahydrofuran (30 ml) was added dropwise to a 2M solution of isopropylmagnesium chloride (11.11 ml, 22.23 mmol) in tetrahydrofuran at 0° C. The solution was stirred for 1 hour at 0° C. and for 1 hour at ambient temperature, then added to diethyl ether which had been saturated with carbon dioxide pellets. After 18 hours water was added to the mixture and the diethyl ether layer separated and discarded. The aqueous layer was acidified to pH 4 with a 2M aqueous solution of hydrochloric acid and extracted with ethyl acetate (100 ml×2). The combined organics were washed with brine then dried over magnesium sulphate, filtered and concentrated under reduced pressure. The solid obtained was triturated with dichloromethane then filtered, to give the title compound (764 mg, 18%); NMR Spectrum: (DMSO-d₆) 2.41 (s, 3H), 8.21 (d, 1H), 8.67 (d, 1H), 13.55 (br s, 1H); Mass Spectrum: M+H⁺ (⁸¹Br) 218.

### Method 10

### Preparation of tert-butyl (2-{[4-(3-ethylpyridin-2-yl)benzoyl]amino}phenyl)carbamate

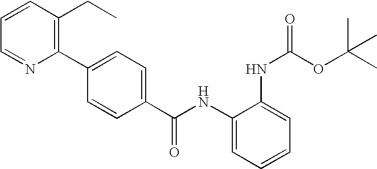

4-(3-Ethylpyridin-2-yl)benzoic acid (Method 11; 84 mg, 0.37 mmol) was taken up in N,N-dimethylformamide (5 ml). 1-(N-t-Butoxycarbonylamino)-2-aminobenzene (87 mg, 0.42 mmol) was then added followed by 4-(4,6-dimethoxy-1,3,5-triazinyl-2-yl)-4-methylmorpholinium chloride (205 mg, 0.74 mmol). The reaction mixture was allowed to stir, at ambient temperature, for 24 hours. Water (50 ml) was then added and the resultant precipitate was collected by suction filtration, washed with more water and dried under vacuum to afford the title compound (139 mg, 90%); NMR Spectrum: (DMSO-d₆) δ1.11 (t, 3H), 1.46 (s, 9H), 2.67 (q, 2H), 7.20 (m, 2H), 7.40 (m, 1H), 7.57 (m, 2H), 7.64 (d, 2H), 7.82 (m, 1H), 8.06 (d, 2H), 8.52 (m, 1H), 8.70 (s, 1H), 9.90 (s, 1H); Mass Spectrum: M+H⁺418.

### Method 11

### Preparation of 4-(3-ethylpyridin-2-yl)benzoic acid

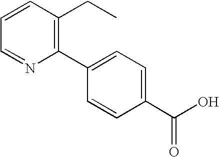

Methyl-4-(3-ethylpyridin-2-yl)benzoate (Method 12; 123 mg, 0.51 mmol) was taken up in a mixture of tetrahydrofuran (1.5 ml), methanol (0.75 ml) and water (0.75 ml). The resultant solution was then treated with lithium hydroxide monohydrate (109 mg, 2.60 mmol) and allowed to stir at ambient temperature for 1 hour. Volatile solvents were then removed by evaporation and remaining aqueous solution was diluted by addition of water (8.5 ml). The solution was then adjusted to pH 4 by addition of 2M aqueous hydrochloric acid and extracted into ethyl acetate (10 ml). The organic phase was separated and the aqueous layer re-extracted with more ethyl acetate (10 ml). The combined organic extracts were washed with brine, dried over magnesium sulphate, filtered and evaporated to afford the title compound (86 mg, 74%); NMR Spectrum: (DMSO-d₆) δ 1.10 (t, 3H), 2.63 (q, 2H), 7.40 (m, 1H), 7.59 (d, 2H), 7.81 (m, 1H), 8.03 (d, 2H), 8.51 (m, 1H), 13.01 (s, 1H); Mass Spectrum: M+H⁺228.

### Method 12

### Preparation of methyl 4-(3-ethylpyridin-2-yl)benzoate

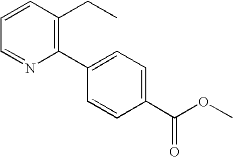

To a solution of methyl 4-(3-vinylpyridin-2-yl)benzoate (Method 13; 134 mg, 0.56 mmol) in ethanol (3 ml) was added palladium (10%) on activated carbon (24 mg). The reaction mixture was evacuated and flushed with nitrogen (×3) before evacuating and flushing with hydrogen (×3). The reaction mixture was then allowed to stir under hydrogen overnight before re-evacuating and flushing with nitrogen (×3). The reaction mixture was filtered through celite, washing with ethanol, and the filtrate evaporated to dryness to afford the title compound (125 mg, 93%); NMR Spectrum: (DMSO-d₆) δ 1.09 (t, 3H), 2.63 (q, 2H), 3.90 (s, 3H), 7.39 (m, 1H), 7.63 (d, 2H), 7.81 (m, 1H), 8.06 (d, 2H), 8.51 (m, 1H); Mass Spectrum: M+H⁺243.

### Method 13

### Preparation of methyl 4-(3-vinylpyridin-2-yl)benzoate

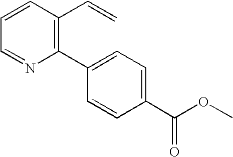

A suspension of methyltriphenylphosphonium iodide (403 mg, 1.00 mmol) in tetrahydrofuran (7 ml) was cooled to 0° C. in an ice bath. Potassium tert-butoxide (113 mg, 1.01 mmol) was then added. The reaction mixture was then allowed to stir for 90 minutes before addition of a solution of methyl 4-(3-formylpyridin-2-yl)benzoate (Method 14; 236 mg, 0.98 mmol) in tetrahydrofuran (3 ml). The mixture was then allowed to warm to ambient temperature with stirring overnight. Water (1 ml) was added before removing volatiles in vacuo. The residue was partitioned between water (10 ml) and dichloromethane (10 ml) and organic layer separated and evaporated. The resultant oil was purified by flash chromatography on silica, eluting with ethyl acetate/isohexane (1:1 v/v) to afford the title compound (134 mg, 57%); NMR Spectrum: (DMSO-d₆) δ 3.90 (s, 3H), 5.41 (m, 1H), 5.93 (m, 1H), 6.69 (m, 1H), 7.47 (m, 1H), 7.67 (d, 2H), 8.07 (d, 2H), 8.14 (m, 1H), 8.62 (m, 1H).

### Method 14

### Preparation of methyl 4-(3-formylpyridin-2-yl)benzoate

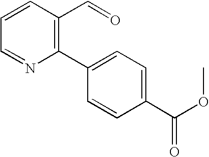

To a solution of 2-chloronicotinaldehyde (1.58 g, 11.17 mmol) and (4-methoxycarbonylphenyl)boronic acid (2.0 g, 11.11 mmol) in 1,2-dimethoxyethane (75 ml) was added 1,1′-bis(diphenylphosphino)ferrocenedichloropalladium(II) dichloromethane adduct (432 mg, 0.53 mmol). The mixture was then purged with nitrogen for 5 minutes before the addition of saturated aqueous sodium bicarbonate solution (30 ml). The reaction mixture was heated to 75° C. with stirring for 2 hours, before cooling back to ambient temperature. The mixture was partitioned between ethyl acetate (200 ml) and water (200 ml). The organic layer was separated, washed with brine, dried over magnesium sulphate, filtered and evaporated to dryness. The resultant solid was purified by flash chromatography on silica, eluting with ethyl acetate and isohexane (1:1 v/v) followed by trituration under isohexane and diethyl ether to afford the title compound (2.07 g, 77%); NMR Spectrum: (DMSO-d₆) δ 3.91 (s, 3H), 7.67 (m, 1H), 7.80 (d, 2H), 8.12 (d, 2H), 8.32 (m, 1H), 8.94 (m, 1H), 9.98 (s, 1H); Mass Spectrum: M+H⁺242.

1. A compound of formula (I): 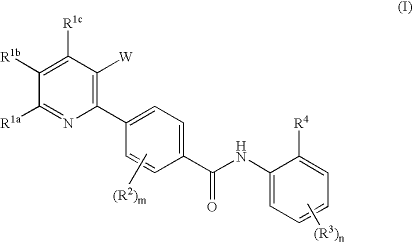
wherein:
R^{1a} is selected from hydrogen, amino, (1-3C)alkyl, N-(1-3C)alkylamino, N,N-di-(1-3C)alkylamino, or a group of the sub-formula II:
R⁵R⁶N—X¹—[CR^{a}R^{b}]_{q}—
wherein:
q is 1, 2 or 3;
each R^{a} and R^{b} group present is independently selected from hydrogen, halo, hydroxy or (1-4C)alkyl;
X¹ is selected from a direct bond or —C(O)—; and
R⁵ and R⁶ are each independently selected from hydrogen or (1-3C)alkyl;
and wherein if R^{1a} is a N-(1-3C)alkylamino or N,N-di-(1-3C)alkylamino group, the (1-3C)alkyl moiety is optionally substituted by hydroxy or (1-2C)alkoxy;
R^{1b} is selected from:
(i) hydrogen, (1-6C)alkyl, halo(1-6C)alkyl, hydroxy(1-6C)alkyl, (3-6C)cycloalkyl, (3-6C)cycloalkyl(1-4C)alkyl, (1-6C)alkoxy, (1-6C)alkoxy(1-6C)alkyl, N-(1-4C)alkylsulphamoyl, N,N-di-[(1-4C)alkyl]sulphamoyl; or
(ii) a group of sub-formula III:
R⁷R⁸N—[CR^{a}R^{b}]ₐ—X²— (III)
wherein:
R⁷ and R⁸ are independently selected from hydrogen, (1-6C)alkyl, (3-6C)cycloalkyl, (3-6C)cycloalkyl(1-6C)alkyl, (1-6C)alkoxy(1-6C)alkyl, (2-3C)alkenyl, (2-3C)alkynyl, or a group of formula IV:
R⁹R¹⁰N—[CR^{a}R^{b}]_{b}—X³— (IV)
wherein:
b is 1, 2 or 3;
R^{a} and R^{b} are as defined above;
X³ is a direct bond or —C(O)—;
R⁹ and R¹⁰ are independently selected from hydrogen, (1-6C)alkyl, (3-6C)cycloalkyl, (3-6C)cycloalkyl(1-6C)alkyl, (1-6C)alkoxy(1-6C)alkyl, or R⁹ and R¹⁰ are linked so that, together with the nitrogen atom to which they are attached, they form a 4-, 5-, 6- or 7-membered heterocyclic ring, said heterocyclic ring optionally comprising, in addition to the nitrogen atom to which R⁹ and R¹⁰ are attached, one or two further heteroatoms selected from N, O or S, and wherein said heterocyclic ring is optionally substituted by one or more hydroxy, halo, (1-4C)alkyl, carbamoyl, oxo, —[CH₂]ₑ—NR¹¹R¹² (wherein e is 0, 1 or 2, and R¹¹ and R¹² are independently selected from hydrogen, (1-6C)alkyl, (3-6C)cycloalkyl or (3-6C)cycloalkyl(1-6C)alkyl);
or R⁷ and R⁸ are linked so that, together with the nitrogen atom to which they are attached, they form a 4-, 5-, 6- or 7-membered heterocyclic ring, said heterocyclic ring optionally comprising, in addition to the nitrogen atom to which R⁷ and R⁸ are attached, one or two further heteroatoms selected from N, O or S, and wherein said heterocyclic ring is optionally substituted by one or more hydroxy, halo, (1-4C)alkyl, carbamoyl, oxo, (2-4C)alkenyl, (2-4C)alkynyl, (1-4C)alkoxy, (1-4C)alkoxy(1-4C)alkyl, (1-4C)alkyl-S(O)_{q}— (where q is 0, 1 or 2), a 5- or 6-membered heterocyclic ring comprising one to three heteroatoms selected from N, O or S or —[CH₂]_{f}—NR¹³R¹⁴ (wherein f is 0, 1 or 2, and R¹³ and R¹⁴ are independently selected from hydrogen, (1-6C)alkyl, (3-6C)cycloalkyl or (3-6C)cycloalkyl(1-6C)alkyl);
X² is selected from a direct bond, —O— or —C(O)—, with the proviso that X² can only be —C(O)— if at least one of R⁷ or R⁸ is a group of formula IV as defined above;
a is 0, 1, 2, 3 or 4;
R^{a} and R^{b} are as defined above; or
(iii) a group of the sub-formula VII:
Q-Z-Y— (VII)
wherein:
Y is a direct bond or —[CR^{a}R^{b}]ₓ—, where integer x is 1 to 4 and R^{a} and R^{b} are as defined above;
Z is absent or selected from —O—, —S—, —SO—, —SO₂—, —NH—SO₂—, —SO₂—NH— or —C(O)—; and
Q is a carbon-linked heterocyclyl or a heterocyclyl-(1-6C)alkyl group, said heterocyclyl or a heterocyclyl-(1-6C)alkyl group being optionally substituted on the heterocyclyl ring by one or more substituent groups (for example 1, 2 or 3), which may be the same or different, selected from halo, oxo, cyano, hydroxy, trifluoromethyl, amino, carboxy, carbamoyl, mercapto, sulphamoyl, (1-3C)alkyl, (2-3C)alkenyl, (2-3C)alkynyl, (1-3C)alkoxy, (1-3C)alkanoyl, (1-3C)alkanoyloxy, (1-3C)alkoxy(1-3C)alkyl, (1-3C)alkoxycarbonyl, halo(1-3C)alkyl, N-[(1-3C)alkyl]amino, N,N-di-[(1-3C)alkyl]amino, N-[(1-3C)alkoxy(1-3C)alkyl]amino, N,N-di-[(1-3C)alkoxy(1-3C)alkyl]amino, N-[(1-3C)alkoxy(1-3C)alkyl]-N-[(1-3C)alkyl]amino, N-(1-3C)alkylcarbamoyl, N,N-di-[(1-3C)alkyl]carbamoyl, (1-3C)alkylthio, (1-3C)alkylsulphinyl, (1-3C)alkylsulphonyl, N-(1-3C)alkylsulphamoyl, N,N-di-[(1-3C)alkyl]sulphamoyl;
R^{1c} is selected from hydrogen, halo, cyano, hydroxy, trifluoromethyl, trifluoromethoxy, amino, carboxy, carbamoyl, mercapto, sulphamoyl, (1-3C)alkyl, (2-3C)alkenyl, (2-3C)alkynyl, (1-3C)alkoxy, (1-3C)alkanoyl, (1-3C)alkanoyloxy, N-(1-3C)alkylamino, N,N-di-[(1-3C)alkyl]amino, (1-3C)alkanoylamino, N-(1-3C)alkylcarbamoyl, N,N-di-(1-3C)alkylcarbamoyl, (1-3C)alkylthio, (1-3C)alkylsulphinyl, (1-3C)alkylsulphonyl, (1-3C)alkoxycarbonyl, N-(1-3C)alkylsulphamoyl, and N,N-di-(1-3C)alkylsulphamoyl;
integer m is 0, 1, 2, 3 or 4;
R² is halo;
integer n is 0, 1, 2, 3 or 4;
R³ is selected from halo, cyano, hydroxy, trifluoromethyl, trifluoromethoxy, amino, carboxy, carbamoyl, mercapto, sulphamoyl, (1-3C)alkyl, (2-3C)alkenyl, (2-3C)alkynyl, (1-3C)alkoxy, (1-3C)alkanoyl, (1-3C)alkanoyloxy, N-(1-3C)alkylamino, N,N-di-[(1-3C)alkyl]amino, (1-3C)alkanoylamino, N-(1-3C)alkylcarbamoyl, N,N-Di(1-3C)alkylcarbamoyl, (1-3C)alkylthio, (1-3C)alkylsulphinyl, (1-3C)alkylsulphonyl, (1-3C)alkoxycarbonyl, N-(1-3C)alkylsulphamoyl, and N,N-di-(1-3C)alkylsulphamoyl;
R⁴ is amino or hydroxy; and
W is methyl or ethyl;
or a pharmaceutically acceptable salt or pro-drug thereof.

2. A compound according to claim 1 wherein R^{1a} is hydrogen.

3. A compound according to claim 1 wherein R^{1b} is selected from hydrogen, or a group of sub-formula (III) as defined above.

4. A compound according to claim 3 wherein R^{1b} is a group of sub-formula (III) as defined in claim 1.

5. A compound according to claim 4 wherein the group of sub-formula (III) is a group
R⁷R⁸N—[CR^{a}R^{b}]ₐ—X²— (III)
where R⁷, R⁸ and X² are as defined in claim 1, a is 1 and R^{a} and R^{b} are both hydrogen.

6. A compound according to claim 4 wherein R⁷ and R⁸ are linked so that, together with the nitrogen atom to which they are attached, they form a 4-, 5-, 6- or 7-membered heterocyclic ring, said heterocyclic ring optionally comprising, in addition to the nitrogen atom to which R⁷ and R⁸ are attached, one or two further heteroatoms selected from N, O or S, and wherein said heterocyclic ring is optionally substituted by one or more hydroxy, halo, (1-4C)alkyl, carbamoyl, oxo, (2-4C)alkenyl, (2-4C)alkynyl, (1-4C)alkoxy, (1-4C)alkoxy(1-4C)alkyl, (1-4C)alkyl-S(O)_{q}— (where q is 0, 1 or 2) a 5- or 6-membered heterocyclic ring comprising one to three heteroatoms selected from N, O or S or —[CH₂]_{f}—NR¹³R¹⁴ (wherein f is 0, 1 or 2 and R¹³ and R¹⁴ are independently selected from hydrogen, (1-6C)alkyl, (3-6C)cycloalkyl or (3-6C)cycloalkyl(1-6C)alkyl).

7. A compound according to claim 6 wherein R⁷ and R⁸ are linked so that they form a 4-, 5- or 6-membered heterocyclic ring which contains one, two or three nitrogen atoms, and wherein said heterocyclic ring is optionally substituted by one or more hydroxy, halo, (1-4C)alkyl carbamoyl, oxo, (2-4C)alkenyl, (2-4C)alkynyl, (1-4C)alkoxy, (1-4C)alkoxy(1-4C)alkyl, (1-4C)alkyl-S(O)_{q}— (where q is 0, 1 or 2), a 5- or 6-membered heterocyclic ring comprising one to three heteroatoms selected from N, O or S or —[CH₂]_{f}—NR¹³R¹⁴ (wherein f is 0, 1 or 2 and R¹³ and R¹⁴ are independently selected from hydrogen, (1-6C)alkyl, (3-6C)cycloalkyl or (3-6C)cycloalkyl(1-6C)alkyl).

8. A compound according to claim 7 wherein the ring formed by R⁷ and R⁸ and the nitrogen atom to which they are attached is substituted by at least one group selected from (1-4C)alkyl, —[CH₂]_{f}—NR¹³R¹⁴ (wherein f is 0, and R¹³ and R¹⁴ are independently selected from (1-6C)alkyl, (2-4C)alkynyl, (1-4C)alkoxy(1-4C)alkyl, or (1-4C)alkyl-S(O)₂—, or a 5- or 6 membered heterocyclic ring comprising from one to three heteroatoms.

9. A compound according to claim 8 wherein the ring formed by R⁷ and R⁸ and the nitrogen atom to which they are attached is substituted by a pyrazinyl group.

10. A compound according to claim 4 wherein R⁷ and R⁸ are independently selected from hydrogen, (1-6C)alkyl, (3-6C)cycloalkyl, (3-6C)cycloalkyl(1-6C)alkyl, (1-6C)alkoxy(1-6C)alkyl, (2-3C)alkenyl, (2-3C)alkynyl, or a group of formula IV:
R⁹R¹⁰N—[CR^{a}R^{b}]_{b}—X³— (IV)
wherein:
b is 2;
R^{a} and R^{b} are as defined above;
X³ is a direct bond or —C(O)—;
R⁹ and R¹⁰ are independently selected from hydrogen, (1-6C)alkyl, or R⁹ and R¹⁰ are linked so that, together with the nitrogen atom to which they are attached, they form a 5-membered heterocyclic ring, said heterocyclic ring optionally comprising, in addition to the nitrogen atom to which R⁹ and R¹⁰ are attached, one or two further heteroatoms selected from N, O or S, and wherein said heterocyclic ring is optionally substituted by hydroxy, halo, (1-4C)alkyl, carbamoyl, oxo, —[CH₂]ₑ—NR¹¹R¹² (wherein e is 0, 1 or 2, and R¹¹ and R¹² are independently selected from hydrogen, (1-6C)alkyl, (3-6C)cycloalkyl or (3-6C)cycloalkyl(1-6C)alkyl).

11. A compound according to claim 10 wherein R⁷ and R⁸ are independently selected from hydrogen, (1-6C)alkyl, (3-6C)cycloalkyl, (3-6C)cycloalkyl(1-6C)alkyl, (1-6C)alkoxy(1-6C)alkyl, (2-3C)alkenyl, or (2-3C)alkynyl.

12. A compound according to claim 11 wherein at least one of R⁷ or R⁸ is (1-6C)alkoxy(1-6C)alkyl.

13. A compound according to claim 10 wherein at least one group R⁷ or R⁸ is a group of sub-formula (IV)
R⁹R¹⁰N—[CR^{a}R^{b}]_{b}—X³— (IV)
wherein:
b is 2;
all R^{a} and R^{b} groups are hydrogen;
X³ is a direct bond or —C(O)—;
R⁹ and R¹⁰ are independently selected from hydrogen, (1-6C)alkyl, or R⁹ and R¹⁰ are linked so that, together with the nitrogen atom to which they are attached, they form a 5-membered heterocyclic ring, said heterocyclic ring optionally comprising, in addition to the nitrogen atom to which R⁹ and R¹⁰ are attached, one or two further heteroatoms selected from N, O or S.

14. A compound according to claim 1 wherein R^{1b} is a group of sub-formula (III) as defined in claim 1 wherein one or R⁷ or R⁸ is hydrogen or (1-6C)alkyl, and the other is (1-6C)alkyl, (3-6C)cycloalkyl, (3-6C)cycloalkyl(1-6C)alkyl, (1-6C)alkoxy(1-6C)alkyl, (2-3C)alkenyl, (2-3C)alkynyl, or a group of formula IV as defined in claim 1.

15. A compound according to claim 1 wherein R^{1c} is hydrogen.

16. A compound according to claim 1 wherein W is methyl.

17. A compound according to claim 1 wherein R⁴ is amino.

18. A compound according to claim 1 wherein m is 0.

19. A compound according to claim 1 wherein n is 0.

20. A compound according to claim 1 which is selected from:
N-(2-aminophenyl)-4-(3-methylpyridin-2-yl)benzamide;
N-(2-aminophenyl)-4-{5-[(4-isopropylpiperazin-1-yl)methyl]-3-methylpyridin-2-yl}benzamide;
N-(2-aminophenyl)-4-(5-{[(cyclopropylmethyl)amino]methyl}-3-methylpyridin-2-yl)benzamide;
N-(2-aminophenyl)-4-{5-[(4-ethylpiperazin-1-yl)methyl]-3-methylpyridin-2-yl}benzamide;
N-(2-aminophenyl)-4-[5-(azetidin-1-ylmethyl)-3-methylpyridin-2-yl]benzamide;
4-(5-amino-3-methylpyridin-2-yl)-N-(2-aminophenyl)benzamide;
N-(2-aminophenyl)-4-{3-methyl-5-[(3-pyrrolidin-1-ylpropanoyl)amino]pyridin-2-yl}benzamide;
6-(4-{[(2-aminophenyl)amino]carbonyl}phenyl)-N-[2-(dimethylamino)ethyl]-N,5-dimethylnicotinamide;
6-(4-{[(2-aminophenyl)amino]carbonyl}phenyl)-N-[2-(dimethylamino)ethyl]-5-methylnicotinamide;
6-(4-{[(2-aminophenyl)amino]carbonyl}phenyl)-5-methyl-N-(2-pyrrolidin-1-ylethyl)nicotinamide;
6-(4-{[(2-aminophenyl)amino]carbonyl}phenyl)-N-[2-(diethylamino)ethyl]-5-methylnicotinamide;
N-(2-aminophenyl)-4-(3-ethylpyridin-2-yl)benzamide;
N-(2-aminophenyl)-4-{3-methyl-5-[(4-methylpiperazin-1-yl)methyl]pyridin-2-yl}benzamide;
N-(2-aminophenyl)-4-(5-{[4-(2-methoxyethyl)piperazin-1-yl]methyl}-3-methylpyridin-2-yl)benzamide;
N-(2-aminophenyl)-4-{3-methyl-5-[(4-prop-2-yn-1-ylpiperazin-1-yl)methyl]pyridin-2-yl}benzamide;
N-(2-aminophenyl)-4-(3-methyl-5-{[3-(methylsulfonyl)pyrrolidin-1-yl]methyl}pyridin-2-yl)benzamide;
N-(2-aminophenyl)-4-(5-{[(2-methoxyethyl)amino]methyl}-3-methylpyridin-2-yl)benzamide;
N-(2-aminophenyl)-4-(5-{[(2-methoxy-1-methylethyl)amino]methyl}-3-methylpyridin-2-yl)benzamide;
N-(2-aminophenyl)-4-(5-{[(2-ethoxyethyl)amino]methyl}-3-methylpyridin-2-yl)benzamide;
N-(2-aminophenyl)-4-(3-methyl-5-{[(2-propoxyethyl)amino]methyl}pyridin-2-yl)benzamide;
N-(2-aminophenyl)-4-(5-{[(2-methoxy-2-methylpropyl)amino]methyl}-3-methylpyridin-2-yl)benzamide;
N-(2-aminophenyl)-4-(5-{[(3-methoxypropyl)amino]methyl}-3-methylpyridin-2-yl)benzamide;
N-(2-aminophenyl)-4-(5-{[(3-ethoxypropyl)amino]methyl}-3-methylpyridin-2-yl)benzamide;
N-(2-aminophenyl)-4-(5-{[(3-isopropoxypropyl)amino]methyl}-3-methylpyridin-2-yl)benzamide;
N-(2-aminophenyl)-4-{3-methyl-5-[(methylamino)methyl]pyridin-2-yl}benzamide;
N-(2-aminophenyl)-4-{5-[(ethylamino)methyl]-3-methylpyridin-2-yl}benzamide;
N-(2-aminophenyl)-4-{3-methyl-5-[(propylamino)methyl]pyridin-2-yl}benzamide;
N-(2-aminophenyl)-4-{5-[(isopropylamino)methyl]-3-methylpyridin-2-yl}benzamide;
N-(2-aminophenyl)-4-{5-[(butylamino)methyl]-3-methylpyridin-2-yl}benzamide;
N-(2-aminophenyl)-4-{5-[(isobutylamino)methyl]-3-methylpyridin-2-yl}benzamide;
N-(2-aminophenyl)-4-{5-[(ethylamino)methyl]-3-methylpyridin-2-yl}benzamide;
N-(2-aminophenyl)-4-(3-methyl-5-{[(2-pyrrolidin-1-ylethyl)amino]methyl}pyridin-2-yl)benzamide;
N-(2-aminophenyl)-4-{3-methyl-5-[(prop-2-yn-1-ylamino)methyl]pyridin-2-yl}benzamide;
N-(2-aminophenyl)-4-(3-methyl-5-{[methyl(prop-2-yn-1-yl)amino]methyl}pyridin-2-yl)benzamide
N-(2-aminophenyl)-4-{3-methyl-5-[(4-pyrazin-2-ylpiperazin-1-yl)methyl]pyridin-2-yl}benzamide;
N-(2-aminophenyl)-4-[3-methyl-5-(pyrrolidin-1-ylmethyl)pyridin-2-yl]benzamide
N-(2-aminophenyl)-4-(5-{[(3S)-3-(dimethylamino)pyrrolidin-1-yl]methyl}-3-methylpyridin-2-yl)benzamide; and
N-(2-aminophenyl)-4-(5-{[(3R)-3-(dimethylamino)pyrrolidin-1-yl]methyl}-3-methylpyridin-2-yl)benzamide.

21. A pharmaceutical composition which comprises a compound according to claim 1, or a pharmaceutically acceptable salt or pro-drug thereof, in association with a pharmaceutically-acceptable diluent or carrier.

22. (canceled)

23. (canceled)

24. (canceled)

25. (canceled)

26. A method for producing a HDAC inhibitory effect in a warm-blooded animal in need of such treatment which comprises administering to said animal an effective amount of a compound of the formula (I), or a pharmaceutically acceptable salt or pro-drug thereof, as defined in claim 1.

27. A process for preparing a compound of formula (I) as defined in claim 1 or a pharmaceutically acceptable salt or pro-drug form thereof, said process comprising a either:
(a) the reaction of a compound of the formula (A) 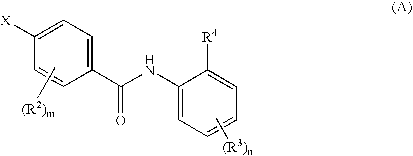
wherein R², R³, R⁴ m and n are as defined in claim 1 and X is a reactive group, with a compound of the formula (B) 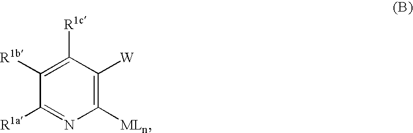
wherein
R^{1a′} is a group R^{1a} as defined in claim 1 or a precursor thereof,
R^{1b′} is a group R^{1b} as defined in claim 1 or a precursor thereof,
R^{1c′} is a group R^{1c} as defined in claim 1 or a precursor thereof,
M is a metal,
L is a ligand, and
integer n′ is 0 to 3;
and wherein if any one of said groups R^{1a′}, R^{1b′} or R^{1c′} is a precursor for a R^{1a}, R^{1b} or R^{1c} group respectively, then said process thereafter comprises a step of converting the compound formed by the reaction of a compound of the formula (A) with a compound of the formula (B) to a compound of formula (I) (by converting the precursor of any one of groups R^{1a}, R^{1b} or R^{1c} group to the appropriate R^{1a}, R^{1b} or R^{1c} group); or
(b) the reaction of a compound of the formula (C) 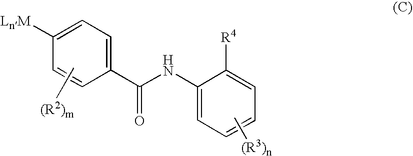
wherein R², R³, R⁴, m and n are as defined in claim 1, M, L and integer n′ are as defined above, with a compound of the formula (D) 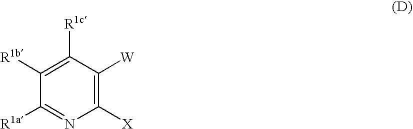
wherein R^{1a′}, R^{1b′} and R^{1c′} are as defined above and X is a reactive group;
and wherein if any one of said groups R^{1a′}, R^{1b′} or R^{1c′} is a precursor for a R^{1a}, R^{1b} or R^{1c} group respectively, then said process comprises an additional step thereafter of converting the compound formed by the reaction of a compound of the formula (C) with a compound of the formula (D) to a compound of formula (I) (by converting the precursor of any one of groups R^{1a}, R^{1b} or R^{1c} group to the appropriate R^{1a}, R^{1b} or R^{1c} group); or
(c) the reaction to couple an acid to an amine, of a compound of the formula (E) 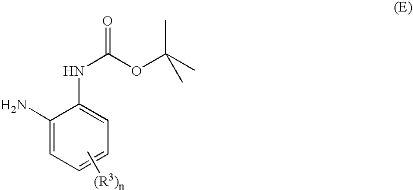
where R³ and n are as defined in claim 1, with a compound of the formula (F) 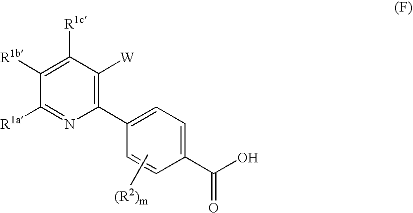
wherein W, R² and m are as defined in claim 1, R^{1a′}, R^{1b′} and R^{1c′} are as defined above, and wherein if any one of said groups R^{1a′}, R^{1b′} or R^{1c′} is a precursor for a R^{1a}, R^{1b} or R^{1c} group respectively, then said process comprises an additional step thereafter of converting the compound formed by the reaction of a compound of the formula (E) with a compound of the formula (F) to a compound of formula (I) (by converting the precursor of any one of groups R^{1a}, R^{1b} or R^{1c} group to the appropriate R^{1a}, R^{1b} or R^{1c} group);
and thereafter if necessary:
i) converting a compound of the formula (I) into another compound of the formula (I); and/or
ii) removing any protecting groups.
